(19) United States
(12) Patent Application Publication
(10) Pub. No.: US 2008/0050398 A1
(43) Pub. Date: Feb. 28, 2008

(54) Composition Comprising Beta-Defensin 2

(76) Inventors: Dirk Bockmuehl, Wuppertal (DE); Heide-Marie Hoehne, Duesseldorf (DE)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/864,386

(22) Filed: Sep. 28, 2007

Related U.S. Application Data
(63) Continuation of application No. PCT/EP2006/002760, filed on Mar. 25, 2006.

(30) Foreign Application Priority Data
Mar. 29, 2005 (DE) 10 2005 014 687.2

(51) Int. Cl. A61K 39/02; A01N 25/00

(52) U.S. Cl. 424/190.1; 424/405

(57) ABSTRACT

The invention relates to compositions comprising at least one peptide with a structure or structural pattern of β-defensin 2 and/or its derivatives, especially human β-defensin 2. The compositions are selected in particular from cosmetic and/or pharmaceutical compositions, cleaning and/or washing agents, water-treatment agents and cooling lubricants. The invention also relates to the use of peptides with a structure or structural pattern of β-defensin 2 and/or its derivatives, especially human β-defensin 2, as an antimicrobial active ingredient in the aforementioned agents.

### CROSS-REFERENCE TO RELATED APPLICATIONS

This application is a continuation of PCT/EP2006/002760 filed Mar. 25, 2006, which claims the benefit of DE 10 2005 014 687.2, filed Mar. 29, 2005, the complete disclosures of which are hereby incorporated by reference in their entirety.

### FIELD OF THE INVENTION

The object of the present invention is compositions comprising at least one peptide having a structure or a structural pattern of β-defensin 2 and/or its derivatives, especially human β-defensin 2. In particular, the compositions are selected from cosmetic and/or pharmaceutical preparations, washing and/or cleaning agents, water-treatment agents and cooling lubricants. A further object of the invention is the use of at least one peptide with a structure or a structural pattern of β-defensin 2 and/or its derivatives, especially human β-defensin 2, as an antimicrobial agent in the means listed.

### BACKGROUND OF THE INVENTION

The disclosures of each patent, patent application, and publication cited or described in this document are hereby incorporated herein by reference, in their entireties.

Use of antimicrobial substances in compositions such as cosmetics, pharmaceutical products, washing and/or cleaning agents, as well as water-treatment agents and cooling lubricants, is known at the state of the art.

If these antimicrobial substances come into contact with human skin in particular, that is usually linked with poor tolerance by the skin or undesired skin reactions, such as irritations, reddenings, and sensitization reactions.

Body odor, for instance, arises from the degradation of constituents of perspiration by bacteria of the skin flora. It is also known that many antibacterial agents have poor action against body odor.

Microbial activity is the cause, or a significant factor in, development of bad skin or pimples as well as dandruff.

Use of antimicrobial agents in cosmetic and/or pharmaceutical preparations can, among other things, reduce dandruff, reduce body odor, improve the status of bad skin, and protect from, or treat, infections, particularly of sensitive and/or irritated skin.

If the antimicrobial agents are used in cosmetic preparations, they usually act unselectively on the organisms in the skin flora and inhibit not only the harmful microorganisms on the skin, but also the useful ones (especially in the face, on the head skin, and under the armpits).

This destruction of the skin flora often results in a possible short-term improvement of the corresponding status of the skin followed, after terminating the treatment, by a reestablishment of the equilibrium of the various skin microorganisms, which can sometimes be shifted toward increased occurrence of pathogenic/harmful microorganisms.

There is a need for new antimicrobial agents or compositions containing them, which do not damage the skin microflora as comprehensively, to be made available.

There is a particular need for making available antimicrobial agents that are more effective against body odor than the agents known so far and that are selectively active against the odor-forming microorganisms.

### SUMMARY OF THE INVENTION

The invention features compositions comprising at least one β-defensin 2 polypeptide. In some aspects, the polypeptide has SEQ ID NO:1, or a derivative thereof, wherein X of SEQ ID NO:1 is selected independently from the group of the essential and non-essential amino acids. X can be Cys, Gly, Thr, or Lys. X can be independently Thr or Gly at position 29, Cys at position 8, 15, 20, 30, 37, or 38, or Lys at position 39 or 40.

In some aspects, the compositions comprise at least one β-defensin 2 polypeptide having SEQ ID NO:2, or a derivative thereof. In some aspects, the compositions comprise at least one β-defensin 2 polypeptide having SEQ ID NO:3, or a derivative thereof.

In the inventive compositions, the β-defensin 2 polypeptide or derivative thereof can be present at a concentration of 10 ng/ml to 100 μg/ml. The β-defensin 2 polypeptide or derivative thereof can be present at a concentration of 0.00001 to 50% by weight.

The invention also features compositions comprising at least one β-defensin 3 polypeptide having SEQ ID NO:4 or 5, or a derivative thereof. In some aspects, the β-defensin 3 polypeptide or derivative thereof is present at a concentration of 0.00001 to 50% by weight.

The inventive compositions can further comprise at least one deodorizing agent or antiperspirant, at least one sebum regulator, at least one anti-inflammatory agent, and/or at least one prebiotic agent.

Also featured are methods for reducing or preventing microbial contamination of washing agents, cleaning agents, cosmetics, pharmaceuticals, filter media, construction materials, or water treatment agents. The methods generally comprise admixing an effective amount of any one or combination of the inventive compositions described and exemplified herein with the washing agents, cleaning agents, cosmetics, pharmaceuticals, filter media, construction materials, or water treatment agents. In some aspects, the cosmetics are deodorants, dental care agents, or oral care agents. In some aspects, the water treatment agents are used to treat water in closed-loop circulation, or to treat water that is used as a cooling lubricant.

Also featured are methods for reducing growth of microbes on the skin, which microbes are harmful to the skin. The methods generally comprise administering to the skin an effective amount of the inventive compositions in combination with at least one prebiotically active agent. In some aspects, the prebiotically active agent is a plant extract, glycerol monoalkyl ether, or ester of an organic acid. In some aspects, the microbes are coagulase-positive Staphylococcus spp., Propionibacterium acnes, Candida albicans, Malassezia furfur, Corynebacterium spp. or Peptostreptococcus spp. The Staphylococcus spp. can be Staphylococcus aureus.

The invention also features methods for reducing the growth of microbes that are harmful to the mouth, comprising exposing the mouth or a dental prosthesis to an effective amount of the inventive compositions in combination with at least one prebiotically active agent as described and exemplified herein.

### DETAILED DESCRIPTION OF ILLUSTRATIVE EMBODIMENTS

By use of molecular biology methods, the following characteristics were found according to the invention to be typical of persons with strong body odor:

a) reduced proportion of Staphylococcus epidermidis

b) increased proportion of Staphylococcus hominis

c) slightly increased proportion of Anaerococcus octavius

d) slightly increased proportion of Corynebacterium species.

Thus there is a need for substances that shift the microflora profile of strong-smelling subjects or of subjects with unpleasant body odor toward the microflora profile of weakly-smelling subjects, especially odor-neutral Staphylococci, promote S. epidermidis in particular, and simultaneously prevent growth of odor-forming Staphylocci, especially of S. hominis and/or Gram-positive anaerobic cocci, especially of especially of Streptococci, primarily of Anaerococcus octavius and/or of odor-forming Corynebacteria and/or of odor-forming Micrococci, primarily of Micrococcus luteus. The minimum requirement for such substances is inhibition of the odor-forming microorganisms without a direct effect on the odor-neutral microorganisms or promotion of the odor-neutral microorganisms without a direct effect on the odor-forming ones.

Therefore it is an objective of the present invention to provide compositions having an antimicrobial action which combat the harmful microorganisms but do not inhibit the useful ones, or harm them less.

In caring for textiles, especially sensitive materials such as silk or microfiber, washing is increasingly done at low temperatures (30 or 40° C.). That generally does not kill the microorganisms, and they remain attached to the items of clothing, so that they can cause infections or reinfections of the skin or mucous membrane with which these items of clothing come into contact.

Because of that, substances with antimicrobial activity, intended to kill the adherent microorganisms, are often added to washing and cleaning agents.

These antimicrobial substances can also remain adherent to the items of clothing, so that they come into contact with human skin, and can cause incompatibility reactions there, such as irritations or sensitization reactions.

A further objective of the invention is to provide washing and/or cleaning agents which eliminate the disadvantages of the state of the art that have been mentioned, and which contain an antimicrobial agent that is compatible with the skin.

Furthermore, circulating water systems, for water treatment, or cooling lubricant systems, are often affected by bacterial contaminants (frequently by bacteria of the genus Pseudomonas and/or other aqueous microorganisms) which should be attacked by adding antimicrobial agents to the cooling lubricant or water-treatment agent in question.

Therefore it is a further objective of the present invention to provide new antimicrobially active cooling lubricants and/or water-treatment agents that comprise intentionally effective antimicrobial agents.

These objectives are attained by compositions containing at least one peptide having a structure or structural pattern of β-defensin 2 according to SEQ ID NO: 1 and/or its derivatives, in which X is selected independently from the group of the essential and nonessential amino acids, especially human β-defensin 2.

The defensins are antimicrobial peptides with short chain length (10 to 50 amino acids) which have been found in, among other places, epithelial tissue of both humans and animals.

In humans, the β-defensins occur primarily in the mucous membranes and epithelial cells, especially in or on the skin. Human β-defensin 1 (hBD1) is found predominantly in the kidneys, saliva, lungs and skin, while human β-defensin 2 (hbd2) is expressed primarily in the skin, the trachea and the lungs if there is a bacterial stimulus.

According to the invention “peptides having a structure or structural pattern of β-defensin 2 according to SEQ ID NO: 1 and/or its derivatives” is understood to be those peptides having a structure or structural pattern according to SEQ ID NO: 1 in which X is selected independently from the group of the essential and nonessential amino acids.

The specified structural pattern can occur one or more times in a peptide according to the invention. Furthermore, one or more other essential or nonessential amino acids can be attached to both ends. It is preferable for a maximum of 15, preferably a maximum of 10, especially 5, and quite particularly preferred one to three essential or nonessential amino acids to be attached to one end or to both ends.

It is especially preferred for X to be selected from C, G, T, K. It is particularly preferred for X at position 29 of the specified sequence to be selected from T (Thr) and G (Gly). It is particularly preferred for the X to be selected independently of each other at positions 8, 15, 20, 30, 37 and 38 of the specified sequence from C (Cys). It is particularly preferred for the X to be selected independently of each other at positions 39 and 40 of the specified sequence from K (Lys). It is especially preferred for the X at positions 39 and 40 of the specified sequence to be selected from K (Lys)

Peptides that contain as the structure or structural pattern that of human β-defensin 2 according to the following formula SEQ ID NO: 2 or are especially preferred.

It is especially preferable to use human β-defensin 2 according to SEQ ID NO: 3. Peptides that contain as the structure or structural pattern the derivative of human β-defensin 2 according to SEQ ID NO: 3 are further especially preferred. It is especially preferable to use the derivative of human β-defensin 2 according to SEQ ID NO: 3.

The peptides can occur as monomers, but can also be clusters of homodimers or heterodimers or trimers.

When reference is made in the text to β-defensin 2 and/or its derivatives, that means especially one of the previously specified peptides or peptides containing the corresponding structural pattern. Peptides that have the exact amino acid sequence according to one of the SEQ ID NOs 1 to 3 are quite especially preferred.

It has been found, surprisingly, that the compositions of at least one peptide with a structure or structural pattern of β-defensin 2 and/or its derivatives, especially human β-defensin 2, exhibit sufficient antimicrobial activity combined with good tolerance by the skin or a very low rate of sensitization.

It was further shown that the compositions containing β-defensin 2 exhibit only a slight inhibitory effect on microorganisms useful to the skin, such as Stapylococcus epidermidis and Bacillus lichenformis. Propionibacterium acnes, on the other hand, is distinctly more strongly inhibited (see example).

Now, surprisingly, substances have been found that promote the growth and/or the physiological activity of S. epidermidis on the skin. Particularly advantageously, the substances can at the same time inhibit the growth and/or the physiological activity of S. hominis, or at least do not promote the growth of S. hominis.

According to a further special embodiment of the present invention, the peptide, or in the case of two or more such peptides, the total of these peptides with a structure or structural pattern of β-defensin 2 and/or its derivatives, especially human β-defensin is contained at concentrations of 10 ng/ml to 100 μg/ml, especially of 50 to 1000 ng/ml, and especially preferably 100 to 400 ng/ml, such as, specifically, 200 ng/ml.

It is especially preferred for the stated concentrations to be contained in the ready-to-use formulation or as the final concentration.

Because of the dilution of many products, the concentrations that result in the desired result in the final product are distinctly lower than the concentrations that the products themselves must contain.

According to a particularly preferred embodiment, the preparations according to the invention comprise, along with at least one peptide comprising β-defensin 2 and/or its derivatives as the structure or structural pattern, also at least one peptide comprising a structure or structural pattern of β-defensin 3, especially human β-defensin 3 and/or its derivatives.

Human β-defensin 3 is an antimicrobial peptide having the following amino acid sequence: SEQ ID NO: 4. In particular, two more lysines can also be attached to this sequence SEQ ID NO: 5.

One particular advantage of such a combination is that they are even more effective against undesired microorganisms or a large number of various undesired microorganisms. The combination according to the invention is used preferably in cosmetic formulations, especially deodorants and/or products for cleaning and/or care of the mouth and/or teeth. In particular they can act especially well there against those microorganisms that occur in the area of mouth and/or teeth care and in the armpits.

According to a particularly preferred embodiment, the compositions according to the invention comprise the peptide having a structure or structural pattern of β-defensin 3, preferably human β-defensin 3, in concentrations of 0.00001 to 50% by weight, in particular of 0.0001 to 10% by weight and especially preferably of 0.0001 to 0.01% by weight.

For washing agents, one must expect a dilution factor (ratio of washing agent concentration to water) of 1:20 to 1:200. The dilution factor for washing agents is frequently between 1:60 and 1:100, such as 1:80.

One skilled in the art will have no problem in calculating the required initial concentration in the optionally concentrated composition.

One particular advantage of the present invention is that the antimicrobial agent can be used in very small proportions, in comparison with other antimicrobially active substances.

According to a preferred embodiment of the present invention the compositions are selected from cosmetic and/or pharmaceutical compositions, washing and/or cleaning agents, water treatment agents and cooling lubricants.

According to a particularly preferred embodiment, the cosmetic and/or pharmaceutical composition is selected from tooth and/or mouth care agents, skin and/or hair care agents, especially deodorants.

Substances also used as ingredients of cosmetic agents are designated in the following according to the International Nomenclature Cosmetic Ingredient (INCI) nomenclature. Chemical compounds have an INCI name in English. Vegetable ingredients are listed solely in Latin according to Linnaeus. So-called ‘trivial’ names such as “water”, “honey” or “sea salt” are likewise given in Latin. The INCI designations can be found in the International Cosmetic Ingredient Dictionary and Handbook, Seventh Edition (1997), which is published by the Cosmetic, Toiletry and Fragrance Association (CTFA), 1101 17th Street NR, Suite 300, Washington, D.C. 20036, USA. It contains more than 9,000 INCI names as well as references to more than 37,000 trade names and technical designations, including their distributors from more than 31 countries. The International Cosmetic Ingredient Dictionary and Handbook assigns the ingredients to one or more chemical classes, such as polymeric ethers, and one or more functions, such as Surfactants—Cleansing agents, which explain them in more detail and which may be referred to in the following. The term CAS means that the subsequent series of numbers is a designation of the Chemical Abstracts Service.

The cosmetic or pharmaceutical composition according to the invention can be any desired administration form, such as a solid or liquid soap, a lotion, a spray, a creme, a gel, an emulsion, a cleaning liquid or cleansing milk, a deodorant, an antiperspirant, a salve, a hair treatment or a shampoo, and it can also be contained in any of the described or other administration forms, such as also in a plaster, especially in a gel-reservoir plaster or a matrix plaster.

A preferred embodiment of the invention comprises other cosmetic agents to increase the effect of the compositions according to the invention even more. Preferred agents include moisture retention agents, especially selected from the water-soluble multifunctional C₂-C₈ alkanols with 2-6 hydroxyl groups and/or the water-soluble polyethylene glycols with 3-20 ethylene oxide units, and mixtures of them. The preferred components are selected from 1,2-propyleneglycol, 2-methyl-1,3-propanediol, glycerol; butylene glycols such as 1,2-butylene glycol, 1,3-butylene glycol, and 1,4-butylene glycol; pentylene glycols, hexanediols such as 1,6-hexanediol, hexanetriols such as 1,2,6-hexanetriol, 1,8-octanediol, dipropylene glycol, tripropylene glycol, diglycerol, triglycerol, erythritol, sorbitol, and mixtures of those substances named above. Suitable water-soluble polyethylene glycols are selected from PEG-3, PEG-4, PEG-6, PEG-7, PEG-8, PEG-9, PEG-10, PEG 12, PEG-14, PEG-16, PEG-18 and PEG-20 and mixtures of them, with PEG-3 to PEG-8 being preferred. Sugars and certain sugar derivatives, such as fructose, glucose, maltose, maltitol, mannitol, inositol, sucrose, trehalose, xylose, rhamnose and fucose are suitable according to the invention. Other preferred moisture retention agents are taurine, allantoin, 2-hydroxyethylurea, Biosaccharide Gum-1 and glycosaminoglycans and their salts and/or esters, especially hyaluronic acid, its salts and its silanol derivatives.

The compositions according to the invention can preferably comprise at least one moisture retention agent in proportions of 0.1-25% by weight, preferably 1.0-15% by weight, especially preferably 5-10% by weight, based on the total composition.

Other preferred active substances are selected from oligomers and polymers of amino acids, N—(C₂-C₂₄)-acylamino acids, the esters and/or the physiologically compatible metal salts of those substances, DNA or RNA oligonucleotides, natural betaine compounds, vitamins, provitamins and vitamin precursors of the groups A, B, C, E, H and K and the esters of the substances previously named, α-hydroxycarboxylic acids, α-ketocarboxylic acids, β-hydroxycarboxylic acids and their esters, lactone or salt forms, flavonoids and flavonoid-rich plant extracts, isoflavonoids and isoflavonoid-rich plant extracts, polyphenols and polyphenol-rich plant extracts, ubiquinone and ubiquinol, as well as their derivatives, naturally occurring xanthine derivatives selected from caffeine, theophylline, theobromine and aminophylline, ectoin, organic and inorganic UV filter substances, auto-tanning substances and skin-lightening substances.

The oligomers of amino acids and/or N—(C₂-C₂₄)-acylamino acids are selected from di, tri, tetra, penta or hexa-peptides that can be acylated and/or esterified. Optionally acylated and/or esterified dipeptides according to the invention are Tyr-Arg, Val-Trp, Asn-Phe, N-palmitoyl-β-Ala-His, carnosine (β-Ala-His) and N-palmitoyl-Pro-Arg. Optionally acylated and/or esterified tripeptides according to the invention are Gly-His-Lys, N-palmitoyl-Gly-His-Lys, Gly-Lys-His, His-Ala-Orn, Lys-Phe-Lys, N-elaidoyl-Lys-Phe-Lys and N-acetyl-Arg-Lys-Arg-NH2. Optionally acylated and/or esterified tetrapeptides according to the invention are Gly-Gln-Pro-Arg (SEQ ID NO:6), Gly-Gln-Arg-Pro (SEQ ID NO:7), and N-palmitoyl-Gly-Gln-Pro-Arg (SEQ ID NO:8). Optionally acylated and/or esterified pentapeptides according to the invention are Lys-Thr-Thr-Lys-Ser (SEQ ID NO:9), N-palmitoyl-Lys-Thr-Thr-Lys-Ser (SEQ ID NO:10) (Matrix from Sederma), N-palmitoyl-Tyr-Gly-Gly-Phe-Met (SEQ ID NO:11), and N-palmitoyl-Tyr-Gly-Gly-Phe-Leu (SEQ ID NO:12). Palmitoyl-Val-Gly-Val-Ala-Pro-Gly (SEQ ID NO:13) (Biopeptide EL from Sederma) is a hexapeptide preferred according to the invention.

It can be particularly preferred according to the invention to use a mixture of at least two oligopeptides. A particularly preferred mixture is the combination of N-palmitoyl-Gly-His-Lys (e.g., Biopeptide CL from Sederma) and N-palmitoyl-Gly-Gln-Pro-Arg (SEQ ID NO:8) (e.g., in Eyeliss from Sederma). A prepared mixture of the tripeptide Palmitoyl-Gly-His-Lys and the tetrapeptide N-palmitoyl-Gly-Gln-Pro-Arg (SEQ ID NO:8) is available under the trade name of Matrixyl 3000, also from Sederma.

The physiologically compatible salts of the agents preferred according to the invention, which contain acid groups and can form salts, are selected from the ammonium, alkali metal, magnesium, calcium, aluminum, zinc and manganese salts. The sodium, potassium, magnesium, aluminum, zinc and manganese salts are preferred.

The polymers of the amino acids and/or of the N—(C₂₋₂₄)-acylamino acids are selected from vegetable and animal protein hydrolysates and/or proteins. Animal protein hydrolysates are, for example, elastin, collagen, keratin, silk and milk albumin protein hydrolysates, which can also occur in the form of salts. Vegetable protein hydrolysates, e.g., soy, wheat, almond, pea, potato and rice protein hydrolysates, are preferred. Corresponding commercial products are, for example, DIAMIN® (Diamalt), GLUADIN® (Cognis), LEXEIN® (Inolex), and CROTEIN® (Croda). Soy protein hydrolysates, such as the commercial product Phytokine from Coletica, or Ridulisse C from Silab, are particularly preferred.

Protein hydrolysates can also, naturally, contain monomeric amino acids and oligopeptides; their compositions are normally not defined.

It is likewise possible to use acyl derivatives of the protein hydrolysates, such as in the form of their fatty acid condensation products. Corresponding commercial products are, for example, LARNEPON® (Cognis), GLUADIN® (Cognis), LEXEIN® (Inolex), CROLASTIN® or CROTEIN® (Croda).

Cationic protein hydrolysates are also usable according to the invention. The preferred cationic protein hydrolysates are those for which the fundamental protein portion has a molecular weight of 100 to 25,000 Dalton, preferably 250 to 5000 Dalton. Cationic protein hydrolysates are further understood to include quaternized amino acids and mixtures of them. Also, the cationic protein hydrolysates can be still further derivatized. Some of the products listed under the INCI designations in the “International Cosmetic Ingredient Dictionary and Handbook” (7th Edition, 1997) and which are commercially available are: Cocodimonium hydroxypropyl hydrolyzed collagen, Steardimonium hydroxypropyl hydrolyzed collagen, Cocodimonium hydroxypropyl hydrolyzed rice protein, Cododimonium hydroxypropyl hydrolyzed silk, Cocodimonium hydroxypropyl hydrolyzed soy protein, Cocodimonium hydroxypropyl hydrolyzed wheat protein, Cocodimonium hydroxypropyl silk amino acids, and hydroxypropyl arginine lauryl/myristyl ether HCl. The vegetable-based cationic protein hydrolysates and derivatives are quite specially preferred.

In a further preferred embodiment the polymers of the amino acids are selected from DNA repair enzymes.

DNA repair enzymes preferred according to the invention are photolyase and T4 endonuclease, the latter of which is abbreviated as T4N5 in the following. Both these enzymes are already known as so-called DNA repair enzymes at the state of the art. DNA repair is understood, according to the definition, to be the splitting or removal of UV-induced pyrimidine dimers from the DNA. Photolyase is the short designation of deoxyribodipyrimidine photolyase or DNA photolyase, an enzyme with the classification number EC 4.1.99.3. A particularly efficient photolyase is derived from Anacystis nidulans, a phototrophic marine microorganism. The photolyase from A. nidulans has since been obtained in technologically relevant quantities from E. coli. Photolyase is activated by light.

The enzyme T4 endonuclease is produced from the denV gene of the T4 bacteriophage. It is one of the phosphodiesterases which hydrolyze nucleic acids at the 5′-3′ bond. T4N5 is active even without the action of light.

Use of liposome-encapsulated DNA repair enzymes is particularly preferred according to the invention. Liposome-encapsulated photolyase is commercially available under the product name of Photosome™ and liposome-encapsulated T4N5, for example, under the designation Ultrasome™ from AGI Dermatics, USA.

The Photosome™ or Ultrasome™ are used in the compositions according to the invention in proportions of 0.1-10% by weight, preferably 0.5-5% by weight, and particularly preferably 1.0-4.0% by weight, based on the complete agent.

The compositions according to the invention comprise the oligomers or polymers of amino acids, N—(C₂₋₂₄)-acylamino acids and/or the esters and/or the physiologically compatible metal salts of those substances in proportions of 0.0001-10% by weight, preferably 0.01 to 5% by weight, and especially preferably 0.1-3% by weight, based in each case on the total composition.

In a further preferred embodiment the compositions according to the invention comprise at least one DNA oligonucleotide or one RNA oligonucleotide. According to the invention, an oligonucleotide is understood to be a polymer of 2 to 20, preferably 2 to 10, mononucleotides that are linked by phosphoric acid diester bridges just as polynucleotides and nucleic acids. The nucleotides consist of nucleobases (usually pyrimidine or purine derivatives), pentoses (usually D-ribofuranose or 2-deoxyribofuranose with a β-N-glycoside bond to the nucleobase) and phosphoric acid. The mononucleotides are, for example, adenosine phosphate, cytidine phosphate, guanosine phosphate, uridine phosphate and thymidine phosphate, specifically CMP (cytidine-5′-monophosphate), UDP (uridine-5′-diphosphate), ATP (adenosine-5′-triphosphate) and GTP (guanosine-5′-triphosphate).

Thymidine dinucleotide is a particularly preferred oligonucleotide according to the invention. The compositions according to the invention comprise the DNA oligonucleotides or RNA oligonucleotides n proportions of 0.0001-5% by weight, preferably 0.001-1.0% by weight, and especially preferably 0.01-0.5% by weight, based on the total composition.

Natural betaine compounds preferred according to the invention are naturally occurring compounds with the atomic grouping R₃N⁺CH₂XCOO— according to IUPAC Rule C-816.1. The so-called ‘betaine surfactants’ (synthetic) are not among the betaine compounds used according to the invention, nor are other zwitterionic compounds in which the positive charge is at N or P and the negative charge is formally at O, S, B or C, but which do not correspond to IUPAC Rule C-816.1. Betaine compounds preferred according to the invention are betaine (Me₃N⁺—CH₂—COO—) and carnitine (Me₃N⁺CH₂CHOHCH₂COO—), where ME=methyl in each case.

The compositions according to the invention comprise the betaine compounds in a total proportion of 0.05 to 5% by weight, preferably 0.1 to 3% by weight, and especially preferably 0.5 to 2% by weight, based on the total composition in each case.

In a further preferred embodiment the compositions according to the invention comprise at least one vitamin, provitamin or compound designated as a vitamin precursor from the vitamin groups A, B, C, E, H and K and the esters of those substances.

The group of substances designated as Vitamin A include retinol (Vitamin A1) and 3,4-dihydroretinol (Vitamin A2). β-carotene is the precursor of retinol. Vitamin A components according to the invention include, for example, Vitamin A acid and its esters, Vitamin A aldehyde, and Vitamin A alcohol and its esters such as retinyl palmitate and retinyl acetate. The compositions according to the invention comprise the Vitamin a components preferably in proportions of 0.05-1% by weight, based on the total composition.

The Vitamin B group, or the Vitamin B complex, includes, among others, Vitamin B1, trivial name thiamine; chemical name 3-[(4′-amino-2′-methyl-5′-pyrimidinyl)-methyl]-5-(2-hydroxyethyl)-4-methylthiazolium chloride. Thiamine hydrochloride is used preferably in proportions of 0.05 to 1% by weight, based on the total composition. Vitamin B2, trivial name riboflavin; chemical name 7,8-dimethyl-10-(1-D-ribityl)-benzo[g]pteridine-2,4(3H,10H)-dione. Riboflavin or its derivatives are used preferably in proportions of 0.05 to 1% by weight, based on the total composition. Vitamin B3. The compounds nicotinic acid and nicotinic acid amide (niacinamide) are listed under this name. Nicotinic acid amide is preferred according to the invention, and is used in the agents according to the invention preferably in proportions of 0.05 to 1% by weight, based on the total composition. Vitamin B5 (pantothenic acid and panthenol). Use of panthenol is preferred. Derivatives of panthenol that can be used according to the invention include, in particular, the ester and ether of panthenol as well as cationically derivatized panthenols. In a further preferred embodiment of the invention, derivatives of 2 furanone having the general structural formula (I) can be used in place of or in addition to pantothenic acid or panthenol.

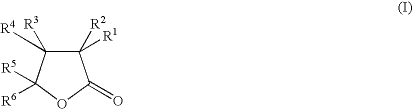

Those 2-furanone derivatives are preferred in which the substituents R¹ to R⁶ represent, independently of each other, a hydrogen atom, a hydroxyl group, a methyl, methoxy, aminomethyl or hydroxymethyl group, a saturated or singly or doubly unsaturated linear or branched C₂-C₄ hydrocarbon group, a saturated or singly or doubly unsaturated linear or branched mono, di or tri-hydroxy-(C₂-C₄)-hydrocarbon group, or a saturated or singly or doubly unsaturated linear or branched mono, di or triamino-(C₂-C₄)-hydrocarbon group. Especially preferred derivatives are the substances dihydro-3-hydroxy-4,4-dimethyl-2(3H)-furanone, with the trivial name Pantolactone (Merck); 4-hydroxymethyl-γ-butyrolactone (Merck), 3,3-dimethyl-2-hydroxy-γ-butyrolactone (Aldrich) and 2,5-dihydro-5-methoxy-2-furanone (Merck), with all their stereoisomers expressly included. Pantolactone (dihydro-3-hydroxy-4,4-dimethyl-2(3H)-furanone) is the 2 furanone derivative extraordinarily preferred according to the invention. In that compound, R¹ in formula (I) stands for a hydroxyl group, R² for a hydrogen atom, R³ and R⁴ for methyl groups and R⁵ and R⁶ for hydrogen atoms. The stereoisomer (R)-pantolactone arises in degradation of pantothenic acid. The named compounds of the Vitamin B5 type, and the 2-furanone derivatives, are comprised in the agents according to the invention in a total proportion of 0.05 to 5% by weight, preferably 0.1 to 3% by weight, and especially preferably 0.5 to 2% by weight, based in each case on the total composition.

Vitamin B6. This is understood not to mean a uniform substance, but the derivatives of 5-hydroxymethyl-2-methylpyrimidin-3-ol known by the trivial names pyridoxine, pyridoxamine and pyridoxal. Vitamin B6 is used in the compositions according to the invention preferably in proportions of 0.0001 to 1.0% by weight, particularly in proportions of 0.001 to 0.01% by weight. Vitamin B7 (biotin), also called Vitamin H or “skin vitamin.” Biotin is (3aS,4S,6aR)-2-ketohexahydrothienol-[3,4d]-imidazol-4-valeric acid. The compositions according to the invention comprise biotin in proportions of 0.0001 to 1.0% by weight, especially in proportions of 0.001 to 0.01% by weight.

Vitamin C (ascorbic acid) is used preferably in proportions of 0.1 to 3% by weight, based on the total composition. It can be preferred to use the derivatives ascorbyl palmitate, ascorbyl stearate, ascorbyl dipalmitate, ascorbyl acetate, magnesium ascorbyl phosphate, sodium ascorbyl phosphate, sodium and magnesium ascorbate, disodium ascorbyl phosphate and disodium ascorbyl sulfate, potassium ascorbyl tocopheryl phosphate, chitosan ascorbate or ascorbyl glucoside. Use in combination with tocopherols can likewise be preferred.

The Vitamin E group includes tocopherol, particularly a tocopherol and its derivatives. Particularly preferred derivatives are the esters, such as tocopheryl acetate, nicotinate, phosphate, succinate, linoleate, oleate, Tocophereth-5, Tocophereth-10, Tocophereth-12, Tocophereth-18, Tocophereth-50 and Tocophersolan. Tocopherol and its derivatives are used preferably in proportions of 0.05 to 1% by weight, based on the total composition.

Vitamin F is usually understood to mean essential fatty acids, especially linoleic acid, linolenic acid and arachidonic acid.

Vitamin H is another name for biotin, or Vitamin B7 (see above).

The fat-soluble vitamins of the Vitamin K group, for which the basic structure is 2-methyl-1,4-naphthoquinone, include phylloquinone (Vitamin K1), famoquinone or menaquinone-7 (Vitamin K2) and menadione (Vitamin K3). Vitamin K is comprised preferably in proportions of 0.0001 to 1.0% by weight, especially 0.01 to 0.5% by weight, based in each case on the total composition.

Vitamin A palmitate (retinyl palmitate), panthenol, pantolactone, nicotinic acid amide, pyridoxine, pyridoxamine, pyridoxal, biotin, ascorbyl palmitate or acetate, magnesium ascorbyl phosphate, sodium ascorbyl phosphate, sodium and magnesium ascorbate, and the tocopherol esters, especially tocopheryl acetate, are especially preferred according to the invention.

In a further preferred embodiment, the compositions according to the invention comprise at least one α-hydroxycarboxylic acid, α-ketocarboxylic acid or β-hydroxycarboxylic acid or their ester, lactone, or salt forms. Suitable α-hydroxycarboxylic acids or α-ketocarboxylic acids according to the invention are glycolic acid, lactic acid, tartaric acid, citric acid, 2-hydroxybutanoic acid, 2,3-dihydroxypropanoic acid, 2-hydroxypentanoic acid, 2-hydroxyhexanoic acid, 2-hydroxyheptanoic acid, 2-hydroxyoctanoic acid, 2 hydroxy-decanoic acid, 2-hydroxydodecanoic acid, 2 hydroxytetradecanoic acid, 2 hydroxyhexadecanoic acid, 2-hydroxyoctadecanoic acid, mandelic acid, 4 hydroxymandelic acid, malic acid, erythroic acid, threoic acid, glucaric acid, galactaric acid, mannaric acid, gularic acid, 2-hydroxy-2-methylsuccinic acid, gluconic acid, pyruvic acid, glucuronic acid and galacturonic acid. Particularly preferred α-hydroxycarboxylic acids are lactic acid, citric acid, glycolic acid and gluconic acid. Salicylic acid is a particularly preferred β-hydroxycarboxylic acid. The esters of the acids named are selected from the methyl, ethyl, propyl, isopropyl, butyl, amyl, pentyl, hexyl, 2-ethylhexyl, octyl, decyl, dodecyl and hexadecyl esters. The α-hydroxycarboxylic acids, α-ketocarboxylic acids or β hydroxycarboxylic acids or their derivatives are comprised in proportions of 0.1-10% by weight, preferably 0.5-5% by weight, based in each case on the total composition.

In a further preferred embodiment the compositions according to the invention comprise at least one flavonoid or at least one flavonoid-rich plant extract.

The flavonoids preferred according to the invention include the glycosides of the flavones, the flavanones, the 3-hydroxyflavones (flavanols), the aurones and the isoflavones. Especially preferred flavonoids are selected from naringin (aurantiine, naringenin-7-rhamnoglucoside), a glucosylrutin, a glucosylmyricetin, α-glucosylisoquercetin, α-glucosylquercetin, hesperidine (3′,5′,7-trihydroxy-4′-methoxyflavanon-7-rhamnoglucoside, hesperitin-7-O-rhamnoglucoside), neohesperidine, rutin (3,3′,4′,5′,7-pentahydroxy-flavon-3 rhamnoglucoside, quercetin-3-rhamnoglucoside), Troxerutin (3,5-dihydroxy-3′,4′,7-tris(2-hydroxyethoxy)-flavon-3-(6-O-(6-deoxy-α-L-mannopyranosyl)-β-D-glucopyranoside)), monoxerutin (3,3′,4′,5-tetrahyxroxy-7-(2-hydroxyethoxy)-flavon-3-(6-O-(6-deoxy-α-mannopyranosyl)-β-D-glucopyranoside)), disomin (3′,4′,7-trihydroxy-5-methoxyflavanon-7-rhamnoglucoside), eriodictin and apigenin-7-glucoside (4′,5,7-trihydroxyflavon-7-glucoside).

Extraordinarily preferred flavonoids according to the invention are α-glucosylrutin, naringin and apigenin-7-glucoside.

Biflavonoids built up of two flavonoid units, which occur in Gingko species, for example, are likewise preferred. Other preferred flavonoids are the chalcones, primarily phloricin and neohesperidine dihydrochalcone.

The flavonoids are preferably used according to the invention in proportions of 0.0001 to 1% by weight, preferably 0.0005 to 0.5% by weight and especially preferably 0.001 to 0.1% by weight, based in each case on the flavonoid-active substance in the total composition.

In a further preferred embodiment the compositions according to the invention comprise at least one isoflavonoid or at least one isoflavonoid-rich plant extract. Here the isoflavones and isoflavone glycosides are included in the isoflavonoids.

In the sense of the present invention, the isoflavones are understood to include the hydrogenation, oxidation or substitution products of 3-phenyl-4H-1-benzopyran, in which a hydrogenation can occur in the 2,3-position of the hydrocarbon skeleton, an oxidation or formation of a carbonyl group can occur in the 4 position, and substitution is understood to mean the replacement of one or more hydrogen atoms by hydroxyl or methoxy groups. The isoflavones preferred according to the invention include, for example, daidzein, genistein, prunetin, biochanin, orobol, santal, pratensein, irigenin, glycitein, biochanin A and formononetin. Especially preferred isoflavones are daidzein, genistein, glycitein and formononetin.

In the isoflavone glycosides preferred according to the invention, the isoflavone is glycosidically linked through at least one hydroxyl group with at least one sugar. Sugars which can be considered include monosaccharides or oligosaccharides, especially D-glucose, d-galactose, D-glucuronic acid, D galacturonic acid, X-xylose, D-apiose, L-rhamnose, L-arabinose and rutinose. Particularly preferred isoflavone glycosides according to the invention are daidzin and genistein. It is further preferred according to the invention for the isoflavones and/or their glycosides contained in the compositions to be mixtures of substances obtained from a plant, especially a plant extract. Such plant substance mixtures can be obtained in the manner well known to persons skilled in the art by pressing or extracting plants such as soy, red clover or chickpeas. Isoflavones or isoflavone glycosides in the form of extracts obtained from soy are used preferably in the preparations according to the invention. They are commercially available, for example, under the product name soy Protein Isolate SPI (Protein Technology International, St. Louis) or Soy Phytochemicals Concentrate SPC (Archer Daniels Midland, Decatur). Apple seed extract, especially the commercial product Ederline from Seporga, is a further especially preferred isoflavonoid-rich plant extract. Ederline comprises phytohormones, isoflavonoids, phytosterols, triterpenoids, tocopherols and natural waxes. The isoflavonoids are used according to the invention in proportions of 0.00001 to 1% by weight, preferably 0.0005 to 0.5% by weight and especially preferably 0.001 to 0.1% by weight, based in each case on the isoflavonoid-active substance in the total composition.

In a further preferred embodiment, the compositions according to the invention additionally comprise at least one polyphenol or a poplyphenol-rich plant extract.

Polyphenols are understood according to the invention to be aromatic compounds containing at least two phenolic hydroxyl groups in the molecule. They include the three dihydroxyphenols pyrocatechol, resorcinol and hydroquinone, as well as phloroglucinol, pyrogallol and hexahydroxybenzene. Free and etherified polyphenols occur in nature in, for example, flower color substances (anthocyanidines, flavones), in tanning agents (catechols, tannins), as components of lichens or ferns (usnic acid, acylpolyphenols), in lignins, and as gallic acid derivatives. Preferred polyphenols are flavones, catechols, usnic acid, and, as tanning agents, the derivatives of gallic acid, digallic acid and digalloylgallic acid. Especially preferred polyphenols are the monomeric catechols, i.e., derivatives of the flavan-3-ols and leukoanthocyanidines, i.e., derivatives of leukoanthocyanidine that preferably bear hydroxyl groups in the 5,7,3′,4′,5′ positions, preferably epicatechol and epigallocatechol, and the tannins derived from them by self-condensation. Such tannins are preferably not used as isolated pure substances, but as extracts from plant parts rich in tannins, such as extracts of catechu, quebracho, oak bark and pine bark, as well as other tree barks, leaves of green tea (Camellia sinensis) and mate. The tannins are likewise especially preferred. One especially preferred polyphenol-rich cosmetic active ingredient is the commercial product Sepivinol R, an extract of red wine, obtainable from the company Seppic. A further especially preferred polyphenol-rich cosmetic active ingredient is the commercial product Crodarom Chardonnay, an extract from the seeds of Chardonnay grapes, obtainable from Croda. The polyphenols are used according to the invention in proportions of 0.001 to 10% by weight, preferably 0.005 to 5% by weight, and especially preferably 0.01 to 3% by weight, based in each case on the total composition.

In a further preferred embodiment the compositions according to the invention comprise at least one ubiquinone or ubiquonol or derivatives of them. Ubiquinols are the reduced forms of the ubiquinones. The ubiquinones preferred according to the invention have the Formula (II):
with n=6, 7, 8, 9 or 10.

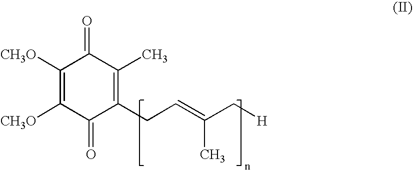

The ubiquinone of Formula (II) with n=10, also known as coenzyme Q10, is especially preferred. The ubiquinones, ubiquinols or their derivatives are used according to the invention in proportions of 0.0001 to 1% by weight, preferably 0.001 to 0.5% by weight, and especially preferably 0.005 to 0.1% by weight, based in each case on the total composition.

In a further preferred embodiment the compositions according to the invention comprise at least one naturally occurring xanthine derivative, selected from caffeine, theophylline, theobromine and aminophylline.

The naturally occurring xanthine derivates are used according to the invention in proportions of 0.0001 to 1% by weight, preferably 0.001 to 0.5% by weight, and especially preferably 0.005 to 0.1% by weight, based in each case on the total composition.

In a further preferred embodiment the compositions according to the invention comprise ectoin. Ectoin is the trivial name for 2-methyl-1,4,5,6-tetrahydropyrimidin-4-carboxylate. Ectoin is used according to the invention in proportions of 0.0001 to 1% by weight, preferably 0.001 to 0.5% by weight, and especially preferably 0.005 to 0.01% by weight, based in each case on the total composition.

In a further preferred embodiment the compositions according to the invention comprise at least one inorganic and/or at least one organic UV filter substance.

The UV filter substances are substances that occur in liquid or crystalline form at room temperature and which are able to absorb ultraviolet radiation and to give off the absorbed radiation in the form of longer-wavelength radiation, such as heat. UVA and UVB filters are distinguished. The UVA and UVB filters can be used either alone or in mixtures. Use of mixtures of filters is preferred according to the invention. The organic UV filters used according to the invention are selected from the physiologically compatible derivatives of dibenzoylmethane, cinnamic acid esters, diphenylacrylic acid esters, benzophenone, camphor, p-aminobenzoic acid esters, o-aminobenzoic acid esters, salicylic acid esters, benzimidazoles, symmetrically or unsymmetrically substituted 1,3,5-triazines, monomeric and oligomeric 4,4-diarylbutadiene-carboxylic acid esters and carboxamides, ketotricyclo(5.2.1.0)decane, benzalmalonic acid esters, benzoxazole and any desired mixtures of the components named. The organic UV filters can be oil-soluble or water-soluble. Oil-soluble UV filters particularly preferred according to the invention are 1-(4-tert-butylphenyl)-3-(4′-methoxyphenyl)propan-1,3-dione (PARSOL 1789®), 1 phenyl-3-(4′-isopropoxyphenyl)-propan-1,3-dione, 3-(4′-methylbenzylidene)-D,L-camphor, 4-(dimethylamino)benzoic acid 2-ethylhexyl ester, 4 (dimethylamino)benzoic acid 2-octyl ester, 4-(dimethylamino)benzoic acid amyl ester, 4-methoxycinnamic acid 2-ethylhexyl ester, 4-methoxycinnamic acid propyl ester, 4-methoxycinnamic acid isopentyl ester, 2-cyano-3,3-phenylcinnamic acid (Octocrylene), salicylic acid 2-ethylhexyl ester, salicylic acid 4-isopropylbenzyl ester, salicylic acid homomethyl ester (3,3,5-trimethyl-cyclohexyl salicylate), 2-hydroxy-4-methoxybenzophenone, 2-hydroxy-4-methoxy-4′-methylbenzophenone, 2,2′-dihydroxy-4-methoxybenzophenone, 4 methoxybenzmalonic acid di-2-ethylhexyl ester, 2,4,6-trianilino-(p-carbo-2′-ethyl-1′-hexyloxy)-1,3,5-triazine (Octyl Triazone, UVINUL® T 150), dimethicodiethylbenzal malonate (CAS No. 207574-74-1, PARSOL® SLX), dioctyl butamido triazone (UVASORB® HEB), 2,4-bis-[5-1-(dimethylpropyl)-benzoxazol-2-yl-(4-phenyl)imino]-6-(2-ethylhexyl)-imino-1,3,5-triazine (CAS No. 288254-16-0, UVASORB® K2A) and arbitrary mixtures of the components named.

Preferred water-soluble UV filters are 2-phenylbenzimidazol-5-sulfonic acid, phenylene-1,4-bis-(2-benzimidazyl)-3,3′,5,5′-tetrasulfonic acid and their alkali, alkaline earth, ammonium, alkylammonium, alkanolammonium and glucammonium salts, sulfonic acid derivatives of benzophenones, preferably 2-hydroxy-4-methoxybenzophenone-5-sulfonic acid, and their salts, sulfonic acid derivatives of 3-benzylidene-camphor, such as 4-(2-keton-3-bornylidene-methyl)benzenesulfonic acid and 2-methyl-5-(2-keto-3-bornylidene)sulfonic acid and their salts.

Some of the oil-soluble UV filters can themselves serve as solvents or solubilizers for other UV filters. Thus, for instance, it is possible to make solutions of the UV-A filter 1-(4-tert.-butylphenyl)-3-(4′methoxyphenyl)-propan-1,3-dione (e.g., PARSOL® 1789 in various UV-B filters. Therefore the compositions according to the invention comprise, in a further preferred embodiment, 1-(4-tert.-butylphenyl)-3-(4′-methoxyphenyl)propan-1,3-dione in combination with at least one UV-B filter, selected from 4-methoxycinnamic acid 2-ethylhexyl ester, 2-cyano-3,3-phenylcinnamic acid 2-ethylhexyl ester, salicylic acid 2-ethylhexyl ester, and 3,3,5-trimethylcyclohexyl salicylate. In this combination, the weight ratio of UV-B filter to 1-(4-tert.-butylphenyl)-3-(4′-methoxyphenyl)propen-1,3-dione is between 1:1 and 10:1, preferably between 2:1 and 8:1, with the molar ratio correspondingly between 0.3 and 3.8, preferably between 0.7 and 3.0, especially preferably about 2.5.

The inorganic light-protection pigments preferred according to the invention are finely disperse or colloidally dispersed metal oxides and metal salts, such as titanium dioxide, zinc oxide, iron oxide, aluminum oxide, cerium oxide, zirconium oxide, silicates (talc) and barium sulfate. The average particle size should be less than 100 nm, preferably between 5 and 50 nm, and especially between 15 and 30 nm, so-called nanopigments. They can have a spherical form, but it is also possible to use particles that deviate from the spherical form in ellipsoidal or other ways. The pigments can also be surface-treated, i.e., hydrophilized or hydrophobized. Typical examples are coated titanium dioxide, such as Titandioxid T 805 (Degussa) or EUSOLEX® T2000 (Merck). The principal hydrophobic coatings are silicones, and especially trialkoxyoctylsilane or simethicone. Titanium dioxide and zinc oxide are particularly preferred.

According to the invention the organic UV filter substances have concentrations of 0.1-30% by weight, preferably 0.5-20% by weight, especially preferably 1.0-15% by weight, and extraordinarily preferably 3.0-10% by weight, based in each case on the total composition.

According to the invention the inorganic UV filter substances have concentrations of 0.1-15% by weight, preferably 0.5-10% by weight, especially preferably 1.0-5% by weight, and extraordinarily preferably 2.0-4.0% by weight, based in each case on the total composition.

In a further preferred embodiment the compositions according to the invention comprise at least one auto-tanning agent. Auto-tanning agents preferred according to the invention are selected from dihydroxyacetone and erythrulose. According to the invention the self-tanning active ingredients comprise proportions of 0.1-15% by weight, preferably 0.5-10% by weight, especially preferably 1.0-5% by weight, and extraordinarily preferably 2.0-4.0% by weight, based in each case on the total composition.

In a further preferred embodiment the compositions according to the invention comprise at least one skin-lightening active ingredient. Skin-lightening active ingredients preferred according to the invention are selected from ascorbic acid, the esters of ascorbic acid with phosphoric acid and/or C₂-C₂₀ carboxylic acids and their alkali and alkaline earth metal salts, kojic acid, hydroquinone, arbutin, mulberry extract and licorice extract, and mixtures of them. The ascorbic acid derivatives and kojic acid are preferred, both as individual substances and in mixtures. Sodium ascorbyl phosphate, magnesium ascorbyl phosphate, ascorbyl monopalmitate, ascorbyl dipalmitate, ascorbyl monostearate, ascorbyl distearate, ascorbyl mono-ethylhexanoate, ascorbyl di-ethylhexanoate, ascorbyl mono-octanoate, ascorbyl di-octanoate, ascorbyl mono-isostearate and ascorbyl di-isostearate. The ascorbic acid derivatives extraordinarily preferred according to the invention are sodium ascorbyl phosphate and magnesium ascorbyl phosphate.

The skin-lightening active ingredients are contained in proportions of 0.05 to 5% by weight, preferably 0.1-2% by weight, based in each case on the total composition.

A further objective of the current invention is use of a cosmetic and/or pharmacological, especially pharmacological, especially pharmacological, especially dermatological topical composition that comprises in a suitable carrier at least β-defensin 2 and/or its derivatives, especially human β-defensin 2 for non-therapeutic cosmetic treatment of sensitive skin, dry skin, unclean skin, atopic dermatitis, aged skin, UV-damaged skin and/or irritated skin.

A further objective of the present invention is a process for non-therapeutic cosmetic skin treatment in which a cosmetic or pharmacological, especially dermatological topical composition that comprises at least one β defensin 2 and/or its derivatives, especially human β-defensin 2, is applied onto the skin especially the facial skin or the axillary region.

The compositions according to the invention optionally comprise at least one conditioning active ingredient along with β-defensin 2 and/or its derivatives, especially human β-defensin 2. ‘Conditioning active ingredient’ means, according to the invention, those substances that are absorbed into keratinic materials, especially into the skin, and improve the physical and sensory properties. Conditioning agents smooth the outermost layer of the skin, and make it soft and supple.

Conditioning active ingredients preferred according to the invention are selected from fats, especially vegetable oils such as sunflower oil, olive oil, soy oil, canola oil, almond oil, jojoba oil, orange oil, wheat germ oil, peach kernel oil, and the liquid portions of coconut oil, lanolin and its derivatives, liquid paraffin oils, isoparaffin oils and synthetic hydrocarbons, di n-alkyl ethers with a total of 12 to 36 C atoms, such as di-n-octyl ether and n hexyl-n-octyl ether; fatty acids, especially linear and/or branched, saturated and/or unsaturated C₆₋₃₀ fatty acids, fatty alcohols, especially saturated, singly or doubly unsaturated, branched or unbranched fatty alcohols having 4-30 C atoms, which can be ethoxylated with 1-75, preferably 5-20 ethylene oxide units and/or with 3-30, preferably 9-14 propylene oxide units; ester oils, i.e., esters of C₆₋₃₀ fatty acids with C₂₋₃₀ fatty alcohols; hydroxycarboxylic acid alkyl esters, dicarboxylic acid esters such as di n-butyl adipate, and diol esters such as ethylene glycol dioleate or propylene glycol di(2-ethylhexanoate), symmetric, unsymmetric or cyclic esters of carbonic acid with fatty alcohols, such as glyceryl carbonate or dicapryl carbonate (CETIOL® CC), mono, di and tri-fatty acid esters of saturated and/or unsaturated linear and/or branched fatty acids with glycerol, which can be ethoxylated with 1-10, preferably 7-9 ethylene oxide units, such as PEG-7 glyceryl cocoate; waxes, especially insect waxes, plant waxes, fruit waxes, ozocerite, microwaxes, ceresin, paraffin waxes, triglycerides of saturated and optionally hydroxylated C₁₆₋₃₀ fatty acids, such as hydrogenated triglyceride fats, phospholipids, such as soy lecithin, egg lecithin and cephalins, silicone compounds, selected from decamethyl cyclopentasiloxane, dodecamethyl cyclohexasiloxane and silicone polymers which may be cross-linked if desired, such as polydialkylsiloxanes, polyalkylarylsiloxanes, ethoxylated and/or propoxylated poly-dialkylsiloxanes with the previous INCI designation of Dimethicone Copolyol, and poly-dialkylsiloxanes containing amine and/or hydroxyl groups, preferably substances with the INCI designations Dimethiconol, amodimethicone or Trimethylsilylamodimethicone.

The proportion of fatty substance used is 0.1-50% by weight, preferably 0.1% by weight, and especially preferably 0.1-15% by weight, based in each case on the total composition.

It is advantageous for the cosmetic or pharmacological, especially dermatological compositions to be in the form of a liquid, flowable, or solid oil/water emulsion, water/oil emulsion, or multiple emulsion, especially an oil/water/oil or water/oil/water emulsion, macroemulsion, mini-emulsion, microemulsion, PIT emulsion, nanoemulsion, Pickering emulsion, hydrodispersion, a hydrogel, a lipogel, a single-phase or multi-phase solution, a foam, a powder, or a mixture with at least one polymer suitable as a medicinal adhesive. The agents can also be administered in anhydrous form, such as an oil or a balm. In this case the carrier can be a vegetable or animal oil, a mineral oil, a synthetic oil, or a mixture of such oils.

In one special embodiment of the invention the compositions are microemulsions. Within this invention, ‘microemulsions’ are understood to be not only the thermodynamically stable microemulsions but also the so-called “PIT” emulsions. These emulsions are systems with the 3 components water, oil and emulsifier, which occur as oil/water emulsions at room temperature. On heating these systems, microemulsions form in a certain temperature range (called the phase inversion temperature or “PIT”), which on further heating change into water/oil emulsions. O/W emulsions form again on subsequent cooling, but even at room temperature they are microemulsions or very finely divided emulsions with average particle diameters less than 400 nm and especially about 100-300 nm. those micro- or “PIT” emulsions that exhibit an average particle diameter of about 200 nm can be preferred according to the invention.

In the embodiment as emulsions, the compositions according to the invention comprise at least one surface-active substance as an emulsifier or dispersing agent. Suitable emulsifiers are, for instance, addition products of 4-30 moles of ethylene oxide and/or 0 to 5 moles of propylene oxide to linear C₈-C₂₂ fatty alcohols, to C₁₂-C₂₂ fatty acids and to C₈-C₁₅ alkylphenols; C₁₂-C₂₂ fatty acid monoesters and diesters of addition products of 1 to 30 moles of ethylene oxide to C₃-C₆ polyols, especially to glycerol, ethylene oxide and polyglycerol addition products to methyl glucoside fatty acid esters; fatty acid alkanolamides and fatty acid glucamides, C₈-C₂₂ alkyl mono- and oligo-glycosides and their ethoxylated analogs, where oligomerization degrees of 1.1 to 5, preferably 1.2 to 2.0, with glucose as the sugar component, are preferred. Mixtures of alkyl-(oligo)-glucosides and fatty alcohols, e.g., the commercially available product MONTANOV® 68, addition products of 5 to 60 moles of ethylene oxide to castor oil and hydrogenated castor oil, partial esters of polyols having 3-6 carbon atoms with saturated C₈-C₂₂ fatty acids, sterols, especially cholesterol, lanosterol, beta-sitosterol, stigmasterol, campesterol and ergosterol, and mycosterols, phospholipids, principally glucose phospholipids, fatty acid esters of sugars and sugar alcohols such as sorbitol, polyglycerol and polyglycerol derivatives, preferably polyglyceryl-2-dipolyhydroxystearate (commercial product: DEHYMULS® PGPH) and polyglyceryl-3-diisostearate (commercial product: LAMEFORM® TGI), as well as linear and branched C₈-C₃₀ fatty acids and their Na, K, ammonium, Ca, Mg and Zn salts.

The agents according to the invention preferably contain the emulsifiers in proportions of 0.1 to 25% by weight, especially 0.5-15% by weight, based in each case on the total composition.

A particularly preferred embodiment comprises at least one nonionic emulsifier with a HLB value of 8 or less. Such suitable emulsifiers are, for example, compounds having the general formula R¹—O—R², in which R¹ is a primary linear alkyl, alkenyl, or acyl group with 20-30 C atoms, and R² is hydrogen, a group having the formula —(CnH₂nO)x-H with x=1 and n=2-4, or a polyhydroxyalkyl group with 4-6 C atoms and 2-5 hydroxyl groups. Other preferred suitable emulsifiers with a HLB value of 8 or below are the addition products of 1 or 2 moles of ethylene oxide or propylene oxide to behenyl alcohol, erucyl alcohol, arachidyl alcohol or even to behenic acid or erucic acid. The monoesters of C₁₆-C₃₀ fatty acids with polyols such as pentaerythritol, trimethylolpropane, diglycerol, sorbitol, glucose or methylglucose are also preferably suitable. Examples of such products are, for instance, sorbitan monobehenate or pentaerythritol monoerucate.

Other suitable additives are thickeners, e.g., natural and synthetic clays and laminar silicates such as bentonite, hectorite, montmorillonite or LAPONITE®, or anionic polymers of acrylic acid, methacrylic acid, crotonic acid, maleic anhydride and 2-acrylamido-2-methylpropane sulfonic acid, in which the acid groups can occur partially or entirely as sodium, potassium, ammonium, monoethanolammonium or triethanolammonium salts, and in which at least one nonionic monomer can be included. Preferred non-ionogenic monomers are acrylamide, methacrylamide, acrylic acid esters, methacrylic acid esters, vinylpyrrolidone, vinyl ethers and vinyl esters. Preferred anionic copolymers are acrylic acid-acrylamide copolymers and in particular, polyacrylamide copolymers with monomers containing sulfonic acid groups. These copolymers can also be cross-linked. SEPIGEL® 305, SIMULGEL® 600, SIMULGEL® NS and SIMULGEL® EG from the company SEPPIC are suitable commercial products. Other specially preferred anionic homopolymers and copolymers are cross-linked and non-cross-linked polyacrylic acids. The commercial product CARBOPOL® is an example of such compounds. A specially preferred anionic copolymer comprises as the monomer up to 80-98% of an unsaturated, optionally substituted C₃₋₆ carboxylic acid or its anhydride, as well as up to 2-20% of optionally substituted acrylic acid esters of saturated C₁₀₋₃₀ carboxlic acids, in which the copolymer can be cross-linked with the previously named cross-linking agents. Corresponding commercial products are PEMULEN® and the CARBOPOL® types 954, 980, 1342 and ETD 2020 (from B. F. Goodrich).

Examples of suitable nonionic polymers are polyvinyl alcohols, which can be partially saponified, such as the commercial product MOWIOL® and vinylpyrrolidone/vinyl ester copolymers and polyvinylpyrrolidone, marketed, for example, by BASF under the trademark LUVISKOL®.

Other suitable additives are antioxidants, preservatives; solvents such as ethanol, isopropanol, ethylene glycol, propylene glycol, propylene glycol monoethyl ether, glycerol and diethylene glycol; adsorbents and fillers, such as talc and VEEGUM®, perfume oils, pigments and dyes to color the agents, substances to adjust the pH, complexing agents such as EDTA, NTA, β alanine diacetic acid and phosphonic acids, propellants such as propane-butane mixtures, pentane, isopentane, isobutene, N₂O, dimethyl ether, CO₂ and air.

In a specially preferred embodiment according to the invention the cosmetic or pharmaceutical composition is a deodorant and/or antiperspirant. The deodorant and/or antiperspirant is preferably a powder, stick, syndet, washing lotion, aerosol spray, pump spray, liquid or gel roll-on applicant, cream, foam, liquid or solid soap, gel, or an impregnated flexible substrate.

Stick cartridges, roll-ons, pumps, tubes, dishes, dispensers, towels, aerosol dispensers or bottles can be used as applicators, depending on the form of the administration.

The skin of each area of the body can be considered as the site of application, especially the head skin, the skin of the feet and hands, and the vaginal mucosa. In a particularly preferred embodiment the site of application is the skin in the axillary region.

The cosmetic or pharmaceutical composition according to the invention can also have other ingredients than those named above. In a preferred embodiment it comprises at least one of the substances listed in the following. It can also comprise any arbitrary combination of the ingredients listed in the following.

In an embodiment according to the invention the composition comprises at least one further plant extract. This plant extract can, for example, be produced by extraction of the whole plant, or also solely by extraction from flowers and/or leaves and/or seeds and/or other parts of the plant. According to the invention the extracts are prepared principally from the meristem, that is, the undifferentiated tissue capable of dividing, and the extracts from special plants such as green tea, witch hazel, chamomile, pansy, peony, aloe vera, horse chestnut, sage, willow bark, cinnamon tree, chrysanthemums, oak bark, nettle, hops, burdock root, horsetail, hawthorn, linden blossoms, almonds, pine needles, sandalwood, juniper, coconut, kiwi, guava, lime, mango, apricot, wheat, melons, orange, grapefruit, avocado, rosemary, birch, birch, beech sprouts, meadow cress, common milfoil, wild thyme, thyme, garden balm, restharrow, hibiscus (Althea), violet, black currant leaves, coltsfoot, cinquefoil, ginseng, ginger root and sweet potato are preferred as further plant extracts. Extracts of algae can also be used advantageously. The alga extracts used according to the invention are derived from green algae, brown algae, red algae or blue-green algae (cyanobacteria). The algae used for extraction can be of natural origin, or obtained by biotechnological processes and are if desired altered from the natural form. The alteration of the organisms can be accomplished by genetic engineering, by culture, or by cultivation in media enriched with selected nutrients. Preferred algal extracts are derived from kelp, blue-green algae, from the green alga Codium tomentosum and from the brown alga Fucus vesiculosus. A particularly preferred algal extract is derived from the blue-green algae of the species Spirulina, which has been cultivated in a medium enriched with magnesium.

The extracts from Spirulina, green tea, aloe vera, meristem, witch hazel, apricot, guava, sweet potato, lime, mango, kiwi, cucumber, mallow, hibiscus and violet are especially preferred as plant extracts. The agent according to the invention can also comprise as additional plant extracts mixtures of more than one, especially of two, different plant extracts.

Water, alcohols, and mixtures of them, for example, are used as extractants to produce the named other plant extracts and to produce the prebiotically active plant extracts. Of the alcohols, lower alcohols such as ethanol and isopropanol are preferred, but particularly multifunctional alcohols such as ethylene glycol, propylene glycol and butylene glycol, both as single extractants and in mixtures with water. Plant extracts on the basis of water/propylene glycol in proportions of 1:10 to 10:1 have proved particularly suitable. Steam distillation is one of the preferred extraction procedures according to the invention. However, the extraction can also optionally be accomplished in the form of dry extraction.

The plant extracts can be used according to the invention both in pure and diluted form. To the extent that they are used in diluted form, they usually comprise about 2-80% by weight active substance and as the solvent the extractant or mixture of extractants used to obtain them. Depending on the choice of extractant, it can be preferable to stabilize the plant extract by adding a solubilizer. Examples of suitable solubilizers include ethoxylation products of optionally hydrogenated animal and vegetable oils. Preferred solubilizers are ethoxylated mono, di and tri-glycerides of C₈₋₂₂ fatty acids with 4-50 ethylene oxide units, such as hydrogenated ethoxylated castor oil, olive oil ethoxylate, almond oil ethoxylate, mink oil ethoxylate, polyoxyethyleneglycol caprylic or capric acid glyceride, polyoxyethylene glycerol monolaurate and polyoxyethylene glycol coconut fatty acid glyceride. It can also be preferred to use mixtures of more than one, especially of two, different plant extracts in the agents according to the invention.

With respect to usability of plant extracts according to the invention, reference is made to the extracts listed in the table beginning on page 44 of the 3rd Edition of the Guidelines for Declaration of Ingredients of Cosmetic Agents, published by the Industrieverband Körperpflege-und Waschmittel e. V [Industrial Association of Body Care and Washing Agents, (registered association) (IKW), Frankfurt.

The cosmetic or pharmaceutical compositions and especially the topical compositions preferred according to the invention, quite specially preferred the deodorant or antiperspirant compositions that comprise β defensin 2 and/or its derivatives according to the invention, especially human β-defensin 2, can also comprise fats. Fats are understood to include fatty acids, fatty alcohols, natural and synthetic cosmetic oil components as well as natural and synthetic waxes, which can be in solid form or liquid in aqueous or oil dispersion.

Linear and/or branched, saturated and/or unsaturated C₈₋₃₀ fatty acids can be used as the fatty acids. C₁₀₋₂₂ fatty acids are preferred. Examples include caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid [cis-octadecenoic acid], linoleic acid, linolenic acid, eleostearic acid, arichidonic acid, gadoleic acid, behenic acid, and erucic acid, as well as their industrial mixtures. Use of stearic acid is particularly preferred. The fatty acids used can bear one or more hydroxyl groups. Preferred example of those are α-hydroxy-C₈-C₁₈ carboxylic acids and 12-hydroxystearic acid. The proportions used are 0.1-15% by weight, preferably 0.5-10% by weight, especially preferred 1-5% by weight, based in each case on the total composition.

Saturated, singly or doubly unsaturated, branched or unbranched fatty alcohols having 6-30, preferably 10-22 and quite particularly preferably 12-22 carbon atoms can be used as the fatty alcohols. Examples of alcohols usable in the sense of the invention include decanol, octanol, octenol, dodecenol, decenol, octadienol, dodecadienol, decadienol, oleyl alcohol, erucyl alcohol, ricinyl alcohol, stearyl alcohol, isostearyl alcohol, cetyl alcohol, lauryl alcohol, myristyl alcohol, arachidyl alcohol, capryl alcohol [n-octanol], capric alcohol [n-decanol], linoleyl alcohol, linolenyl alcohol and behenyl alcohol, as well as their Guerbet alcohols.

Waxes are often used for stick formulations. Natural or synthetic waxes that can be used according to the invention are solid paraffins or isoparaffins, plant waxes such as candelilla wax, carnauba wax, esparto grass wax, Japan wax, cork wax, sugar cane wax, ouricury wax, montan wax, sunflower wax, fruit waxes and animal waxes, such as beeswax and other insect waxes, spermaceti, shellac wax, wool wax and rump fat, and also mineral waxes such as ceresin and ozocerite, or the petrochemical waxes such as petrolatum, paraffin wax, microwaxes of polyethylene or polypropylene, and polyethylene glycol waxes. It can be advantageous to use hydrogenated or solidified waxes. Chemically modified waxes are also usable, especially the hard waxes, such as montan ester waxes, Sasol waxes, and hydrogenated jojoba waxes.

The mono, di and tri-glycerides of saturated and optionally hydroxylated C₁₆₋₃₀ fatty acids are also suitable, such as hydrogenated triglyceride fats (hydrogenated palm oil, hydrogenated coconut oil, hydrogenated castor oil), glyceryl monostearate (CUTINA® MD), glyceryl tribehenate or glyceryl tri-12-hydroxystearate, as well as synthetic full esters of fatty acids and glycols (e.g. SYNCROWACHS®) or polyols with 2-6 C atoms, fatty acid monoalkanolamides with a C₁₂₋₂₂ acyl group and a C₂₋₄ alkanol group, esters of saturated and/or unsaturated, branched and/or unbranched alkane carboxylic acids having a chain length of 1 to 80 C atoms and saturated and/or unsaturated, branched and/or unbranched alcohols having a chain length of 1 to 80 C atoms, including, for example, synthetic fatty acid—fatty alcohol esters such as stearyl stearate or cetyl palmitate, esters of aromatic carboxylic acids, dicarboxylic acids or hydroxycarboxylic acids (e.g., 12-hydroxystearic acid) and saturated and/or unsaturated, branched and/or unbranched alcohols having a chain length of 1 to 80 C atoms, lactides of long-chain hydroxycarboxylic acids and full esters of fatty alcohols and dicarboxylic or tricarboxylic acids such as dicetyl succinate or dicetyl/stearyl adipate, and mixtures of those substances as long as the individual wax components or mixtures of them are solid at room temperature.

It is particularly preferred that the wax components be selected from the group of esters of saturated unbranched alkane carboxylic acids having a chain length of 14 to 44 C atoms and saturated unbranched alcohols having a chain length of 14 to 44 C atoms, as long as the wax components or the totality of the wax components are solid at room temperature. The wax components can be selected particularly advantageously from the group of C₁₆₋₃₆ alkyl stearates, the C₁₀₋₄₀ alkyl stearates, the C₂₋₄₀ alkyl isostearates, the C₂₀₋₄₀ dialkyl esters of dimer acids, the C₁₈₋₃₈ alkyl hydroxystearoylstearates, the C₂₀₋₄₀ alkyl erucates. C₃₀₋₅₀ alkyl beeswax and cetearyl behenate are also usable. Silicone waxes, e.g., stearyl trimethylsilane/stearyl alcohol, are optionally advantageous. Particularly preferred wax components are the esters of saturated monofunctional C20-C₆₀ alcohols and saturated C₈-C₃₀-monocarboxylic acids. A C₂₀-C₄₀ alkyl stearate obtainable as KESTERWACHS® K82H from Koster Keunen, Inc., is particularly preferred. The wax or the wax components should be solid at 25° C. but melt in the range of 35-95° C., with a range of 45-85° C. preferred. Natural, chemically modified, and synthetic waxes can be used alone or in combination.

The compositions comprise the wax components in a proportion of 0.1 to 40% by weight, based in each case on the total composition, preferably 1-30% by weight and especially 5-15% by weight.

The compositions according to the invention can also comprise at least one polar or nonpolar oil which can be natural or synthetic. The polar oil components can be selected from vegetable oils, e.g., sunflower oil, olive oil, soy oil, canola oil, almond oil, jojoba oil and the liquid parts of coconut oil, as well as synthetic triglyceride oils, from ester oils, i.e., the esters of C₆₋₃₀ fatty acids with C₂₋₃₀ fatty alcohols, from dicarboxylic acid esters such as di-n-butyl adipate, di-(2-ethylhexyl)adipate and di-(2-ethylhexyl)succinate, as well as diol esters such as ethylene glycol dioleate and propylene glycol di(2 ethylhexanoate), from symmetric, unsymmetric or cyclic esters of carboxylic acids with fatty alcohols, such as are described in German Laid-Open Patent Application 197 56 454, glyceryl carbonate or dicaprylyl carbonate (CETIOL® CC), from mono, di and tri-fatty acid esters of saturated and/or unsaturated linear and/or branched fatty acids with glycerol, from branched alkanols, e.g. Guerbet alcohols with a single branch at carbon 2, such as 2-hexyldecanol, 2-octyldodecanol, isotridecanol and isohexadecanol, from alkanediols, such as the vicinal diols that can be obtained from epoxyalkanes having 12-24 C atoms by ring opening with water, from ether alcohols, e.g., the monoalkyl ethers of glycerol, of ethylene glycol, of 1,2-propylene glycol or 1,2-butanediol, from dialkyl ethers each with 12-24 C atoms, such as the alkyl methyl ethers or di-n-alkyl ethers each with a total of 12-24 C atoms, especially di-n-octyl ether (CETIOL® OE from Cognis), as well as from addition products of ethylene oxide and/or propylene oxide to monofunctional or multifunctional C₃₋₂₀ alkanols such as butanol and glycerol, such as PPG-3 myristyl ether (WITCONOL® APM), PPG-14 butyl ether (UCON FLUID® AP), PPG-15 stearyl ether (ARLAMOL® E), PPG-9 butyl ether (BREOX® B25 and PPG-10 butanediol (MACOL® 57). The nonpolar oil components can be selected from liquid paraffin oils, isoparaffin oils, e.g., isohexadecane and isoeicosane, from hydrogenated polyalkenes, especially poly-1-decenes (commercially available as Nexbase 2004, 2006 or 2008 FG (Fortum, Belgium), from synthetic hydrocarbons, such as 1,3-di-(2-ethylhexyl)-cyclohexane (CETIOL® S), and from volatile and nonvolatile silicone oils which can be cyclic, such as decamethylcyclopentasiloxane and dodecamethyl-cyclohexasiloxane, or linear, such as linear dimethylpolysiloxane, commercially available, for instance, as Dow CORNING® 190, 200, 244, 245, 344 or 345 and BAYSILON® 350 M.

The compositions according to the invention can further comprise at least one water-soluble alcohol. ‘Water solubility’, according to the invention, means that at least 5% by weight of the alcohol gives a clear solution at 20° C. or—in the case of long-chain or polymeric alcohols—can be brought into solution at 50 to 60° C. Depending on the form of administration, monofunctional alcohols such as ethanol, propanol or isopropanol are suitable. Water-soluble polyols are also suitable. Those include water-soluble diols, triols, and multifunctional alcohols and polyethylene glycols. Among the diols, C₂-C₁₂ diols are suitable, especially 1,2-propylene glycol, butylene glycols such as 1,2-butylene glycol, 1,3-butylene glycol and 1,4-butylene glycol, and hexanediols such as 1,6-hexandediol. Glycerol, and especially diglycerol and triglycerol, 1,2,6-hexanetriol and dipropylene glycol and the polyethylene glycols (PEG) PEG-400, PEG-600, PEG-1000, PEG-1550, PEG-3000 and PEG 4000.

The proportion of the alcohol or alcohol mixture in the compositions according to the invention is 1-50 or 1-70% by weight, preferably 5-40 or 5-55% by weight, based in each case on the total composition. Both a single alcohol and a mixture of more than one alcohols can be used according to the invention.

The compositions according to the invention can be essentially anhydrous, that is, comprising not more than 5% water by weight, preferably not more than 1% water by weight. In administration forms that contain water, the water content is 5-98% by weight, preferably 10-90 and especially preferably 15-85% by weight, based in each case on the total composition.

The compositions according to the invention can further comprise at least one hydrophilically modified silicone. They make it possible to formulate highly transparent compositions, reduce the stickiness, and leave a fresh feeling for the skin. ‘Hydrophilically modified silicone’ according to the invention is understood to mean polyorganosiloxanes with hydrophilic substituents that cause the silicones to be water-soluble. ‘Water-solubility’ means, according to the invention, that at least 2% by weight of the silicone modified by hydrophilic groups dissolves in water at 20° C. Correspondingly hydrophilic substituents are, for example, hydroxyl, polyethylene glycol or polyethylene glycol/polypropylene glycol side chains, and ethoxylated ester side chains. Preferred as suitable according to the invention are hydrophilically modified silicone copolyols, especially dimethicone copolyols, commercially available, for example, from Wacker-Chemie under the names Belsil® DMC 6031, Belsil® DMC 6032, Belsil® DMC 6038 or Belsil® DMC 307 VP, or from Dow Corning under the name DC 2501. Use of Belsil® DMC 6038 is especially preferred, as it makes it possible to formulate highly transparent compositions that have high acceptance for consumers. ABIL EM97 from Degussa/Goldschmid can also be used as the hydrophilic silicone derivative. An arbitrary mixture of the silicones names can also be used according to the invention.

The proportion of the hydrophilically modified silicone or of the alcohol mixture in the compositions according to the invention is 0.5-10% by weight, preferably 1-8% by weight, and especially preferably 2-6% by weight, based on the total weight of the composition.

The compositions according to the invention can further comprise emulsifiers and/or surfactants. A particularly preferred embodiment involves addition products of 10-40 moles of ethylene oxide to linear or branched fatty alcohols with 16-22 C atoms, to fatty acids with 12-22 C atoms, to fatty acid alkanolamides, to fatty acid monoglycerides, to sorbitan fatty acid monoesters, to fatty acid alkanolamides, to fatty acid glycerides, e.g., to hydrogenated castor oil, to methylglucoside monofatty acid esters and mixtures of those. Essentially, though, any other desired emulsifiers and/or surfactants can be used.

Examples of emulsifiers usable in this sense according to the invention are addition products of 4 to 30 moles of ethylene oxide and/or 0 to 5 moles of propylene oxide to linear or branched C₈-C₂₂ fatty alcohols, to C₁₂-C₂₂ fatty acids and to C₈-C₁₅ alkylphenols. C₁₂-C₂₂ fatty acid monoesters and diesters of addition products of 1 to 30 moles of ethylene oxide to C₃-C₆ polyols, especially to glycerol; Ethylene oxide and polyglycerol addition products to methylglucoside fatty acid esters, fatty acid alkanolamides and fatty acid glucamides; C₈-C₂₂ alkyl mono- and oligo-glycosides and their ethoxylated analogs, with oligomerization degrees of 1.1 to 5, especially 1.2 to 2.0, and glucose as the sugar component are preferred; mixtures of alkyl-(oligo)-glucosides and fatty alcohols, such as the commercially available product MONTANOV® 68; addition products of 5 to 60 moles of ethylene oxide to castor oil and hydrogenated castor oil; partial esters of polyols with 3-6 carbon atoms with saturated C₈-C₂₂ fatty acids; Sterols (sterins). Sterols are understood to be a group of steroids having a hydroxyl group on Carbon 3 of the steroid skeleton, which are isolated both from animal tissue (zoosterols) and plant fats (phytosterols). Examples of zoosterols are cholesterol and lanosterol. Examples of suitable phytosterols are beta-sitosterol, stigmasterol, campesterol and ergosterol. Sterols, called mycosterols, are also isolated from yeasts and molds; phospholipids, especially the glucose phospholipids, which are obtained, for example, as lecithins or phosphatidylcholines from, e.g., egg yolk or plant seeds (e.g., soybeans); fatty acid esters of sugars and sugar alcohols such as sorbitol; polyglycerols and polyglycerol derivatives, preferably polyglyceryl-2-di-polyhydroxystearate (commercial product: DEHYMULS® PGPH) and polyglyceryl-3-di-isostearate (commercial product LAMEFORM® TGI); and, linear and branched C₈-C₃₀ fatty acids and their sodium, potassium, ammonium, calcium, magnesium and zinc salts.

The means according to the invention preferably contain the emulsifiers in proportions of 0.1 to 25% by weight, especially 0.5-15% by weight, based in each case on the total composition.

Another likewise preferred embodiment comprises at least one ionic emulsifier, selected from anionic, zwitterionic, ampholytic and cationic emulsifiers. Preferred anionic emulsifiers are alkyl sulfates, alkyl polyglycol ether sulfates and ether carboxylic acids with 10 to 18 C atoms in the alkyl group and up to 12 glycol ether groups in the molecule, sulfosuccinic acid mono and di-alkyl esters with 8 to 18 C atoms in the alkyl group, and sulfosuccinic acid monoalkyl polyoxyethyl esters with 8 to 18 C atoms in the alkyl group and 1 to 6 ethoxy groups, monoglyceride sulfates, alkyl and alkenyl ether phosphates, and protein-fatty acid condensates. Zwitterionic emulsifiers have at least one quaternary ammonium group and at least one —COO— or —SO₃— group in the molecule. Especially suitable zwitterionic emulsifiers are the so-called ‘betaines’ such as N-alkyl-N,N-dimethylammonium glycinate, N-acyl-aminopropyl-N,N-dimethylammonium glycinate and 2-alkyl-3-carboxymethyl-3-hydroxyethylimidazolines with 8 to 18 C atoms in the alkyl or acyl group, and coco-acylaminoethyl-hydroxyethylcarboxymethyl glycinate.

Ampholytic emulsifiers contain, aside from a C₈-C₂₄ alkyl or acyl group, at least one free amino group and at least one —COO— or —SO₃— group in the molecule, and can form internal salts. Examples of suitable ampholytic emulsifiers are N-alkylglycines, N-alkylaminopropionic acids, N alkylaminobutyric acids, N-alkyliminodipropionic acids, N-hydroxyethyl-N-alkylamidopropylglycines, N-alkyltaurines, N-alkylsarcosines, 2 alkylaminopropionic acids and alkylaminoacetic acids, each with about 8 to 24 C atoms in the alkyl group.

The ionic emulsifiers are used preferably in a proportion of 0.01 to 5% by weight, preferably 0.05 to 3% by weight, and especially preferably 0.1 to 1% by weight, based on the complete agent.

Examples of nonionic surfactants usable according to the invention are: alkoxylated fatty acid alkyl esters having the formula R¹—CO—(OCH₂CHR₂)xOR³, in which the R¹CO stands for a linear or branched, saturated and/or unsaturated acyl group with 6 to 22 carbon atoms, R² stands for hydrogen or methyl, R³ stands for a linear or branched alkyl group with 1 to 4 carbon atoms, and x stands for numbers from 1 to 20; addition products of ethylene oxide to fatty acid alkanolamides and fatty amines; fatty acid N-alkylglucamides; C₈-C₂₂-alkylamine-N-oxide; alkyl polyglycosides corresponding to the general formula RO-(Z)x in which R stands for a C₈-C¹⁶ alkyl group, Z stands for sugar, and x indicates the number of sugar units. The alkyl polyglycosides usable according to the invention may have only one certain alkyl group R. Usually, though, these compounds are produced from natural fats and oils or mineral oils. In this case the alkyl groups R are mixtures corresponding to the starting compounds or corresponding to the particular processing of those compounds. Those alkyl glycosides are particularly preferred for which R is made up essentially of C₈ and C₁₀ alkyl groups, essentially of C₁₂ and C₁₄ alkyl groups, essentially of C₈ to C₁₆ alkyl groups or essentially of C₁₂ to C₁₆ alkyl groups; Any desired monosaccharide or oligosaccharide can be used as the sugar building block Z. Usually sugars with 5 or 6 carbon atoms, and the corresponding oligosaccharides, are used, such as glucose, fructose, galactose, arabinose, ribose, xylose, lyxose, allose, altrose, mannose, gulose, idose, talose and sucrose. Glucose, fructose, galactose, arabinose and sucrose are preferred sugar building blocks. Glucose is especially preferred. The alkyl polyglycosides usable according to the invention contain, on the average, 1.1 to 5, preferably 1.1 to 2.0, especially preferably 1.1 to 1.8 sugar units. The alkoxylated homologs of the alkylpolyglycosides named can also be used according to the invention. These homologs can contain an average of up to 10 ethylene oxide and/or propylene oxide units per alkyl glycoside unit.

Surface active compounds that have at least one quaternary ammonium group and at least one —COO— or —SO₃— group in the molecule can be considered as zwitterionic surfactants. Especially suitable zwitterionic surfactants are the so-called betaines such as N-alkyl-N,N-dimethylammonium glycinate, such as cocoalkyldimethylammonium glycinate and N acylaminopropyl-N,N-dimethylammonium glycinate, for example coco-acylaminopropyldimethylammonium glycinate and 2-alkyl-3-carboxymethyl-3-hydroxyethylimidazoline, each with 8 to 18 C atoms in the alkyl or acyl group, and coco-acylaminoethylhydroxyethyl-carboxymethyl glycinate. The fatty acid amide derivative with the INCI designation Cocamidopropyl Betaine is a preferred zwitterionic surfactant.

All the anionic surface-active substances suitable for use on the human body are suitable as anionic surfactants in the compositions according to the invention. They are characterized by an anionic group, such as a carboxylate, sulfate, sulfonate or phosphate group that makes them water-soluble, and a lipophilic alkyl group with about 8 to 30 C atoms. The molecule can also contain glycol or polyglycol ether groups, ester, ether, amide and hydroxyl groups. Examples of suitable foaming anionic surfactants, each in the form of the sodium, potassium and ammonium, or the mono, di, and trialkanolamine salts with 2 to 4 carbon atoms in the alkanol group are: acyl glutamates of formula (III),

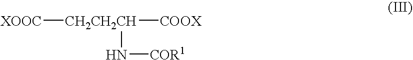

in which the R¹CO stands for a linear or branched acyl group with 6 to 22 carbon atoms and 0, 1, 2 or 3 double bonds, and X stands for hydrogen, an alkali and/or alkaline earth metal, ammonium, alkylammonium, alkanolammonium or glucammonium, such as acylglutamate, which can be derived from fatty acids with 6 to 22, preferably 12 to 18 carbon atoms, such as C12/14 or C12/18 cocofatty acid, lauric acid, myristic acid, palmitic acid and/or stearic acid, especially sodium N-cocoyl- and sodium N-stearoyl-L-glutamate; esters of a hydroxyl-substituted di- or tri-carboxylic acid of the general formula (IV)

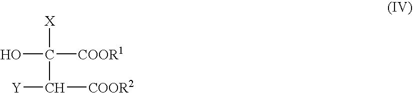

in which x is H or a —CH₂COOR group; y is H or —OH, under the condition that Y═H if X=—CH₂COOR; R¹ and R², independently of each other, are a hydrogen atom, an alkali or alkaline earth metal cation, an ammonium group, the cation of an ammonium-organic base or a group Z which is derived from a polyhydroxylated organic compound selected from the group of etherified (C₆-C₁₈)-alkylpolysaccharides with 1 to 6 monomeric saccharide units and/or the etherified aliphatic (C₆-C₁₆)-hydroxyalkylpolyols with 2 to 16 hydroxyl groups, with the specification that at least one of the groups R, R¹ or R² is a group Z; esters of a sulfosuccinic acid salt having the general formula (V),

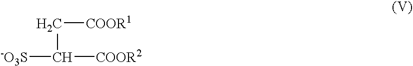

in which R¹ and R², independently of each other, are a hydrogen atom, an alkali or alkaline earth metal cation, an ammonium group, the cation of an ammonium-organic base or a group Z that is derived from a polyhydroxylated organic compound selected from the group of etherified (C₆-C₁₈)-alkylpolysaccharides with 1 to 6 monomeric saccharide units and/or the etherified aliphatic (C₆-C₁₆)-hydroxyalkylpolyols with 2 to 16 hydroxyl groups, with the specification that at least one of the groups R, R¹ or R² is a group Z; sulfosuccinic acid monoalkyl and dialkyl esters with 8 to 24 C atoms in the alkyl groups and sulfosuccinic acid monoalkyl polyoxyethyl esters with 8 to 24 C atoms in the alkyl group and 1 to 6 ethoxy groups; esters of tartaric and citric acids with alcohols which are addition products of about 2-15 molecules of ethylene oxide and/or propylene oxide to fatty alcohols with 8 to 22 C atoms; linear and branched fatty acids with to 30 C atoms (soaps); ether carboxylic acids of the formula R—O—(CH₂—CH₂—O)x-CH₂—COOH, in which R is a linear alkyl group with 8 to 30 C atoms and x=0 or 1 to 16; acylsarcosinates with a linear or branched acyl group having 6 to 22 carbon atoms and 0, 1, 2 or 3 double bonds; acyltaurates with a linear or branched acyl group having 6 to 22 carbon atoms and 0, 1, 2 or 3 double bonds; acyl isethionates with linear or branched acyl group having 6 to 22 carbon atoms and 0, 1, 2 or 3 double bonds; linear alkanesulfonates with 8 to 24 C atoms; linear alpha-olefin sulfonates with 8 to 24 C atoms; alpha-sulfofatty acid methyl esters of fatty acids with 8 to 30 C atoms; alkyl sulfates and alkyl polyglycol ether sulfates having the formula R—O—(CH₂—CH₂O)_{z}—SO₃X, in which X is a preferably linear alkyl group with 8 to 30 C atoms, especially preferably with 8 to 18 c atoms, z=0 or 1 to 12, and X is a sodium, potassium, magnesium, zinc, or ammonium ion, or a mono-alkanol-, dialkanol-, or trialkanol-ammonium ion with 2 to 4 carbon atoms in the alkanol groups, with a particularly preferred example being zinc cocoyl ether sulfate with a degree of ethoxylation of z=3; mixtures of surface-active hydroxysulfonates according to DE-A-37 25 030; sulfated hydroxyalkylpolyethylene and/or hydroxyalkylene propylene glycol ethers according to DE-A-37 23 354; sulfonates of unsaturated fatty acids with 8 to 24 C atoms and 1 to 6 double bonds, according to DE-A-39 26 344; alkyl and/or alkylene ether phosphates having Formula (VI),

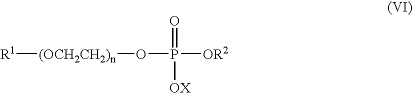

in which R¹ stands preferably for an aliphatic hydrocarbon group with 8 to 30 carbon atoms, R² stands for hydrogen, a group (CH₂CH₂O)nR¹, or X, n stands for numbers 1 to 10 and X for hydrogen, an alkali or alkaline earth metal or NR³R⁴R⁵R⁶, with R³ to R⁶ independently standing for a C1 to C4 hydrocarbon group; sulfated fatty acid alkylene glycol esters having the formula R⁷CO(AlkO)nSO₃M, in which the R⁷CO— stands for a linear or branched, aliphatic, saturated and/or unsaturated acyl group having 6 to 22 C atoms, Alk stands for CH₂CH₂, CHCH₃CH₂, and/or CH₂CHCH₃, n stands for numbers from 0.5 to 5, and M stands for a cation such as is described in German Laid-Open Patent Application DE-OS197 36 906.5; monoglyceride sulfates and monoglyceride ether sulfates having the formula (VII),

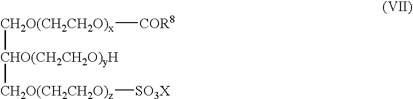

in which the R⁸CO stands for a linear or branched acyl group with 6 to 22 carbon atoms; x, y and z, in total, stand for 0 or for numbers from 1 to 30, preferably 2 to 10; and X stands for an alkali or alkaline earth metal. Typical examples of monoglyceride (ether) sulfates that are suitable in the sense of the invention are the reaction products of lauric acid monoglyceride, coco fatty acid monoglyceride, palmitic acid monoglyceride, stearic acid monoglyceride, oleic acid monoglyceride and tallow fatty acid monoglycerides, and their ethylene oxide adducts, with sulfur trioxide and/or chlorosulfonic acid, in the form of their sodium salts. It is preferable to use monoglyceride sulfates having formula (VI) in which R⁸CO stands for a linear acyl group with 8 to 18 carbon atoms.

The compositions according to the invention can further comprise at least one protein hydrolyzate or its derivative. Both vegetable and animal protein hydrolyzates can be used according to the invention. Animal protein hydrolyzates are, for instance, elastin, collagen, keratin, silk and milk albumin protein hydrolyzates, which can also occur in the form of salts. Plant protein hydrolyzates, such as soy, wheat, almond, pea, potato and rice protein hydrolyzates are preferred according to the invention. Corresponding commercial products are, for instance, DIAMIN® (Diamalt), GLUADIN® (Cognis), LEXEIN® (Inolex) and CROTEIN® (Croda).

Amino acid mixtures obtained in a different manner can be used in place of protein hydrolyzates. Individual amino acids and their physiologically compatible salts can also be used. The amino acids preferred according to the invention are glycine, serine, threonine, cysteine, asparagine, glutamine, pyroglutamic acid, alanine, valine, leucine, isoleucine, proline, tryptophan, phenylalanine, methionine, aspartic acid, glutamic acid, lysine, arginine and histidine, as well as the zinc salts and the acid addition salts of the amino acids named.

It is likewise possible to use derivatives of protein hydrolyzates, in the form of their fatty acid condensation products, for example. Corresponding commercial products are, for example, LAMEPON® (Cognis), GLUADIN® (Cognis), LEXEIN® (Inolex), CROLASTIN® or CROTEIN® (Croda).

Cationized protein hydrolyzates are also usable according to the invention. The original protein hydrolyzate can be derived from animals, from plants, from marine life forms, or from protein hydrolyzates obtained by biotechnology. Those protein hydrolyzates are preferred for which the original protein portion has a molecular weight of 100 to 25,000 Dalton, preferably 250 to 5000 Dalton. Cationic protein hydrolyzates are also understood to mean quaternized amino acids and mixtures of them. The cationic protein hydrolyzates can also be further derivatized. Some of the products named under the INCI designations in “International Cosmetic Ingredient Dictionary and Handbook”, (Seventh Edition, 1997, The Cosmetic, Toiletry and Fragrance Association, 1101 17th Street N. W., Suite 300, Washington D.C. 20036-4702), and commercially available, can be listed: Cocodimonium hydroxypropyl hydrolyzed collagen, cocodimonium hydroxypropyl hydrolyzed casein, steardimonium hydroxypropyl hydrolyzed collagen, steardimonium hydroxypropyl hydrolyzed hair keratin, lauryldimonioum hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, hydroxypropyl arginine lauryl/myristyl ether HCl, and hydroxypropyltrimonium gelatin. The plant-derived cationic protein hydrolyzates and derivatives are quite specially preferred.

The protein hydrolyzates and their derivatives, or the amino acids and their derivatives, are contained in the compositions according to the invention proportions of up to 10% by weight, based on the complete agent. Proportions of 0.1 to 5% by weight, especially 0.1 to 3% by weight, are especially preferred.

The compositions according to the invention can furthermore comprise at least one monosaccharide, oligosaccharide or polysaccharide or their derivatives.

Suitable monosaccharides according to the invention are, for example, glucose, fructose, galactose, arabinose, ribose, xylose, lyxose, allose, altrose, mannose, gulose, idose and talose, the deoxysugars fucose and rhamnose, and amino sugars such as glucosamine or galactosamine. Glucose, fructose, galactose, arabinose and fucose are preferred. Glucose is especially preferred.

Suitable oligosaccharides according to the invention are made up of two to ten monosaccharide units, e.g., sucrose, lactose or trehalose. Sucrose is a specially preferred oligosaccharide. The use of honey, which comprises overwhelmingly glucose and sucrose, is likewise specially preferred.

Suitable polysaccharides according to the invention are made up of more than ten monosaccharide units. Preferred polysaccharides are the starches built up of α-D-glucose units, as well as starch degradation products such as amylase, amylopectin and dextrins. Chemically and/or thermally modified starches such as hydroxypropyl starch phosphate, dihydroxypropyl starch phosphate or the commercial product DRY FLO® are specially advantageous according to the invention. Also preferred are dextrans and their derivatives, such as dextran sulfate. Nonionic cellulose derivatives such as methylcellulose, hydroxypropylcellulose or hydroxyethylcellulose are likewise preferred, as are the cationic cellulose derivatives, such as the commercial products CELQUAT® and POLYMER JR®, and preferably CELQUAT® H 100, CELQUAT® L 200 and POLYMER JR® 400 (Polyquaternium-10) and Polyquaternium-24. Polysaccharides of fucose units, such as the commercial product FUCOGEL® are other preferred examples. The polysaccharides made up of amino-sugar units, especially chitins and their deacylated derivatives, the chitosans, and mucopolysaccharides are especially preferred. The mucosaccharides preferred according to the invention include hyaluronic acid and its derivatives, such as sodium hyaluronate or dimethylsilanol hyaluronate, as well as chondroitin and its derivatives, such as chondroitin sulfate.

In an advantageous embodiment, the compositions according to the invention comprise at least one film-forming, emulsion-stabilizing, thickening or adhesive polymer, selected from natural and synthetic polymers that can be cationic, anionic, amphoterically charged or nonionic. Cationic, anionic and nonioic polymers are preferred according to the invention.

Of the cationic polymers, polysiloxanes with quaternary groups, such as the commercial product Q2-7224 (Dow Corning), DOW CORNING® 929 Emulsion (with amodimethicone), SM-2059 (General Electric), SLM-55067 (Wacker) and ABIL®-Quat 3270 and 3272 (Degussa) are preferred. Preferred anionic polymers, that can support the effect of the active ingredients used according to the invention, comprise carboxylate and/or sulfonate groups, and as monomers, for instance, acrylic acid, methacrylic acid, crotonic acid, maleic anhydride and 2-acrylamido-2-methylpropanesulfonic acid. The acid groups can occur partly or completely as sodium, potassium, ammonium, monoethanolammonium salt or triethanolammonium salt. Preferred monomers are 2 acrylamido-2-methylpropanesulfonic acid and acrylic acid. Quite specially preferred anionic polymers comprise as the sole monomer or as a comonomer 2-acrylamido-2-methylpropanesulfonic acid, in which the sulfonic acid group can be partially or completely in the salt form. In this embodiment, it is preferred to use copolymers of at least one anionic monomer and at least one nonionic monomer. See the substances listed above with respect to the anionic monomers. Preferred nonionogenic monomers are acrylamide, methacrylamide, acrylic acid esters, methacrylic acid esters, vinylpyrrolidone, vinyl ethers and vinyl esters. Preferred anionic copolymers are acrylic acid-acrylamide copolymers, and in particular, polyacrylamide copolymers with monomers containing sulfonic acid groups. A particularly preferred anionic copolymer consists of 70 to 55 mole-percent acrylamide and 30 to 45 mole-percent 2-acrylamido-2-methylpropanesulfonic acid, in which the sulfonic acid groups may be partially in the form of sodium, potassium, ammonium, monoethanolamine or triethanolamine salts. These copolymers can also be cross-linked, with polyolefinically unsaturated compounds preferred as cross-linking agents, such as tetraallyloxyethane, allylsucrose, allylpentaerythritol and methylene-bisacrylamide being used. One such polymer is contained in the commercial product SEPIGEL® 305 from the company SEPPIC. Use of this compound has proved to be particularly advantageous with respect to the teaching of the invention. Sodium acryloyldimethyltaurate copolymers marketed as a compound with isohexadecane and Polysorbate-80 under the name SIMULGEL® 600 have also proved especially effective according to the invention.

Other preferred homopolymers and copolymers are cross-linked and non-cross-linked polyacrylic acids. Allyl ethers of pentaerythritol, of sucrose, and of propylene can be preferred cross-linking agents. The commercial product CARBOPOL® is an example of such compounds. A particularly preferred anionic copolymer contains 80-98% of an unsaturated, optionally substituted C₃₋₆-carboxylic acid or its anhydride, as well as 2-20% of optionally substituted acrylic acid esters of saturated C10-30-carboxlic acids, and the copolymer can be cross-linked with the cross-linking agents named above. Corresponding commercial products are PEMULEN® and the CARBOPOL® types 954, 980, 1342 and ETD 2020 (from B. F. Goodrich).

Suitable nonionic polymers are, for example, polyvinyl alcohols which can be partially saponified, such as the commercial product MOWIOL®, as well as vinylpyrrolidone/vinyl ester copolymers and polyvinylpyrrolidones, such as are marketed under the trade name LUVISKOL® (BASF).

The compositions according to the invention can further comprise at least one α-hydroxycarboxylic acid or α-ketocarboxylic acid or their esters, lactones, or salt forms. Suitable α-hydroxycarboxylic acids or α-ketocarboxylic acids are selected from lactic acid, tartaric acid, citric acid, 2-hydroxybutanoic acid, 2,3-dihydroxypropanoic acid, 2-hydroxypentanoic acid, 2 hydroxyhexanoic acid, 2-hydroxyheptanoic acid, 2-hydroxyoctanoic acid, 2 hydroxydecanoic acid, 2-hydroxydodecanoic acid, 2-hydroxytetradecanoic acid, 2-hydroxyhexadecanoic acid, 2-hydroxyoctadecanoic acid, mandelic acid, 4-hydroxymandelic acid, malic acid, erythroic acid, threoic acid, glucaric acid, galactaric acid, mannaric acid, gularic acid, 2-hydroxy-2-methylsuccinic acid, gluconic acid, pyruvic acid, glucuronic acid and galacturonic acid. The esters of the acids named are selected from the methyl, ethyl, propyl, isopropyl, butyl, amyl, pentyl, hexyl, 2-ethylhexyl, octyl, decyl, dodecyl and hexadecyl esters. The α-hydroxycarboxylic acids or α-ketocarboxylic acids or their derivatives are contained in proportions of 0.1-10% by weight, preferably 0.5-5% by weight, based in each case on the total composition.

The agents according to the invention can comprise other active ingredients, auxiliary ingredients and additives, for example: allantoin; bisabolol; antioxidants, such as imidazoles (e.g., urocanic acid) and their derivatives, peptides such as D,L-carnosine, D-carnosine, L-carnosine and their derivatives (e.g., anserine), chlorogenic acid and its derivatives, lipoic acid and its derivatives (e.g., dihydrolipoic acid), autothioglucose, propylthiouracil and other thiols (e.g., thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters), and their salts, dilauryl thiodipropionate, distearyl thiodipropionate, thiodipropionic acid and its derivatives (esters, ethers, peptides, lipids, nucleotides, nucleosides and salts) as well as sulfoximine compounds (e.g., buthionine sulfoximine, homocysteine sulfoximine, buthionine sulfone, penta, hexa, or heptathionine sulfoximine) in very low tolerable dosages (e.g., picomoles to micromoles per kilogram), also (metal) chelators (e.g., α-hydroxyfatty acids, palmitic acid, phytic acid, lactoferrin), humic acid, gallic acid, gall extracts, bilirubin, biliverdin, EDTA, EGTA and their derivatives, unsaturated fatty acids and their derivatives (e.g., γ-linolenic acid, linoleic acid, oleic acid), folic acid and its derivatives, ubiquinone and ubiquinol and their derivatives, the coniferyl benzoate from gum benzoin, rutic acid and its derivatives, α-glycosylrutin, ferulic acid, furfurylidene glucitol, carnosine, butylhydroxytoluene, butylhydroxyanisole, nor-dihydroguaiacic acid, nor-dihydroguaiaretic acid, trihydroxybutyrophenone, uric acid and its derivatives, catalase, superoxide dismutase, zinc and its derivatives, (e.g., ZnO, ZnSO₄), selenium and its derivatives (e.g., selenomethionine), stilbene and its derivatives (e.g., stilbene oxide, trans-stilbene oxide) and the derivatives of those materials that are suitable as antioxidants (salts, esters, sugars, nucleotides, nucleosides, peptides and lipids); ceramides and pseudoceramides; triterpenes, especially triterpenoic acids such as ursolic acid, rosmarinic acid, betulinic acid, boswellic acid, and bryonolic acid; monomeric catechols, especially catechol and epicatechol, leukoanthocyanidines, catechol polymers (catechol tanning agents) and gallotannins; thickeners, such as gelatins, plant gums such as agar-agar, guar gum, alginates, xanthan gum, gum Arabic, gum karaya or carob seed gum, natural and synthetic clays and laminar silicates, such as bentonite, hectorite, montmorillonite or LAPONITE®, completely synthetic hydrocolloids such as polyvinyl alcohol, and also calcium, magnesium or zinc soaps of fatty acids; plant glycosides; structurants such as maleic acid and lactic acid; dimethylisosorbide; alpha, beta and gamma cyclodextrins, especially to stabilize retinol; solvents, swelling agents and penetrants such as ethanol, isopropanol, ethylene glycol, propylene glycol, propylene glycol monomethyl ether, glycerol and diethylene glycol, carbonates, bicarbonates, guanidine, urea, and primary, secondary and tertiary phosphates; perfume oils, pigments and coloring agents to color the agent; substances to adjust the pH, such as α- and β-hydroxycarboxylic acids; complexing agents such as EDTA, NTA, β-alaninediacetic acid and phosphonic acids; opacifiers such as latex, styrene/PVP and styrene/acrylamide copolymers; pearlescence agents such as ethylene glycol monostearate and distearate, and PEG-3-distearate; propellants such as propane-butane mixtures, N₂O, dimethyl ether, CO₂ and air; MMP-1-inhibiting substances, especially selected from photolyase and/or T4 endonuclease V, propyl gallate, precocenes, 6-hydroxy-7-methoxy-2,2-dimethyl-1(2H)-benzopyran and 3,4-dihydro-6-hydroxy-7-methoxy-2,2-dimethyl-1(2H)-benzopyran; organic, mineral, and/or modified mineral light-protection filters, especially UVA filters and/or UVB filters.

The oral, dental and/or dental prosthesis care agents according to the invention can, for instance, be mouth washes, gels, liquid dentifrice lotions, stiff toothpastes, chewing gums, denture cleaners, or denture adhesives.

It is advantageous that the oral, dental and/or dental prosthesis care agents exhibit especially good action. In particular, killing or inhibition of the microorganisms harmful for the oral flora (especially Streptococcus mutans, S. salivarius, S. mitis, Porphyromonas gingivalis, Treponema denticola, Fusobacterium nucleatum, Actinomyces naeslundii) together with a surprisingly slight to nonexistent action on the microorganisms favorable for the oral flora (such as, in particular, Streptococcus thermophilus) result in such compositions being outstandingly suitable for oral and dental care. For that purpose it is necessary that the compositions according to the invention comprise a suitable carrier.

Powdered compositions of aqueous alcoholic solutions, for example, can serve as carriers. As mouthwashes, they comprise 0 to 15% by weight ethanol, 1 to 1.5% by weight aromatic oils and 0.01 to 0.5% by weight sweeteners; or, as mouthwash concentrates, they comprise 15 to 60% by weight ethanol, 0.05 to 5% aromatic oils, 0.1 to 3% by weight sweeteners, and optionally other auxiliary substances and are diluted with water before use. In that case the concentrations of the components must be chosen high enough that the concentrations after dilution are not lower than the specified lower limits of the concentrations for the application.

Gels and more or less flowable pastes can also serve as carriers which are pressed out of flexible plastic containers or tubes and are applied to the teeth with a toothbrush. Such products comprise higher proportions of moisture-retention agents and binders or consistence regulators and polishing components. Furthermore, these compositions also comprise aromatic oils, sweeteners and water.

The compositions according to the invention can also comprise glycerol, sorbitol, xylitol, propylene glycols, polyethylene glycols or mixtures of those polyols, especially those polyethylene glycols having molecular weights from 200 to 800 (from 400-2000), used as moisture retention agents. Sorbitol is preferred as a moisture retention agent in a proportion of 25-40% by weight.

The compositions according to the invention can also comprise condensed phosphates in the form of their alkali salts, preferably in the form of their sodium or potassium salts, as anticalculus agents and as demineralization inhibitors. The aqueous solutions of these phosphates are alkaline because of hydrolytic effects. The pH of the oral, dental and/or dental prosthesis care agents are adjusted to the preferred value of 7.5-9 by addition of acid.

Mixtures of various condensed phosphates or even hydrated salts of the condensed phosphates can also be used. However, the specified proportions of 2-12% by weight refer to the anhydrous salts. The composition preferably comprises a sodium or potassium tripolyphosphate in a proportion of 5-10% by weight.

An anti-caries fluorine compound, preferably from the group of fluorides or monofluorophosphates, in a proportion of 0.1-0.5% by weight fluorine, is a preferred active ingredient. Suitable fluorine compounds are, for example, sodium monofluorophosphate (Na₂PO₃F), potassium monofluorophosphate, sodium or potassium fluoride, tin fluoride, or the fluoride of an organic amino compound.

Substances used as binders and consistency regulators include, for example, natural and synthetic water-soluble polymers such as carrageen, tragacanth, guar, starches and their nonionogenic derivatives such as hydroxypropyl guar, hydroxyethyl starch, cellulose ethers such as hydroxyethylcellulose or methylhydroxypropylcellulose. Also agar-agar, xanthan gum, pectins, water-soluble carboxyvinyl polymers (e.g., CARBOPOL® types), polyvinyl alcohol, polyvinylpyrollidone, higher-molecular-weight polyethylene glycols (molecular weight 103 to 106 D). Other substances suitable for viscosity control are laminar silicates such as montmorillonite clays, colloidal thickening silicic acids, such as Aerogel silicic acid or pyrogenic silicic acids.

All the substances known as polishing agents can be used as polishing components, but preferably precipitated and gelled silicic acids, aluminum hydroxide, aluminum oxide trihydrate, insoluble sodium metaphosphate, calcium pyrophosphate, calcium hydrogen phosphate, dicalcium phosphate, chalk, hydroxyapatite, hydrotalcite, talc, magnesium aluminum silicate (VEEGUM®), calcium sulfate, magnesium carbonate, magnesium oxide, sodium aluminum silicates, such as Zeolite A, or organic polymers, such as polymethacrylate. The polishing agents are used preferably in low proportions, such as 1-10% by weight.

The organoleptic properties of the dental and/or oral care products can be improved by adding aromatic oils and sweetening agents. All the natural and synthetic aroma substances commonly used for oral, dental and/or dental prosthesis care agents can be considered as aroma oils. Natural aroma substances can be both in the form of the ethereal oils isolated from the drugs or the individual components isolated from them. Preferably they should comprise at least one aroma oil from the group of peppermint oil, spearmint oil, anise oil, caraway seed oil, fennel oil, eucalyptus oil, cinnamon oil, geranium oil, sage oil, thyme oil, marjoram oil, citrus oil, wintergreen oil, or one or more synthetically produced components of those oils isolated from them. The major components of the oils named are, for example, menthol, carvone, anethol, cineol, eugenol, cinnamaldehyde, geraniol, citronellol, linalool, salvene, thymol, terpenes, terpinol, methylchavicol and methyl salicylate. Other suitable aroma agents are, for example, menthyl acetate, vanillin, ionone, linolyl acetate, rhodinol and piperidone. Suitable sweeteners are either natural sugars such as sucrose, maltose, lactose and fructose, or synthetic sweeteners such as saccharin sodium, sodium cyclamate, or Aspartame.

Alkyl and/or alkenyl (oligo)-glycosides are usable as surfactants. Their production and use as surface-active substances are known, for example, from U.S. Pat. No. 3,839,318, U.S. Pat. No. 3,707,535, U.S. Pat. No. 3,547,828, DE-A-19 43 689, DE-A-20 36 742 and DE-A-30 01 164 and EP-A-77 167. With respect to the glycoside group, both monoglycosides (x=1), in which a pentose or hexose group is bound glycosidically to a primary alcohol with 4 to 16 C atoms and oligomeric glycosides with a degree of oligomerization x up to 10 are suitable. Here the degree of oligomerization is a statistical average, based on the usual homolog distribution for such industrial products.

An alkyl- and/or alkenyl-(oligo)-glycoside having the formula RO(C₆H₁₀O)x-H, in which R is an alkyl and/or alkenyl group with 8 to 14 C atoms and x is an average value from 1 to 4 is preferably suitable. Alkyl-oligo-glucosides based on hydrogenated C_{12/14} Coco alcohol with a degree of polymerization of 1 to 3 are especially preferred. The alkyl and/or alkenyl glycoside surfactant can be used very sparingly, with proportions of only 0.005 to 1% by weight being sufficient.

The compositions according to the invention can comprise, in addition to the alkylglucoside surfactants named, other nonionic, ampholytic and cationic surfactants, such as: fatty alcohol polyglycol ether sulfates, monoglyceride sulfates, monoglyceride ether sulfates, mono and/or di-alkyl sulfosuccinates, fatty acid isethionates, fatty acid sarcosinates, fatty acid taurides, fatty acid glutamates, ether carboxlic acids, fatty acid glucamides, alkylamidobetaines and/or protein-fatty acid condensates, the latter preferably based on wheat proteins. A nonionic solubilizer from the group of surface-active compounds may be necessary, especially to solubilize the aroma oils, which are usually insoluble in water. Ethoxylated fatty acid glycerides, ethoxylated fatty acid sorbitan partial esters of fatty acid partial esters of glycerol or sorbitol ethoxylates are particularly suitable for this purpose, for example. Solubilizing agents from the group of ethoxylated fatty acid glycerides comprise primarily addition products of 20 to 60 moles of ethylene oxide to mono- and di-glycerides of linear fatty acids having 12 to 18 C atoms or to triglycerides of hydroxyfatty acids such as oxystearic acid or ricinoleic acid. Other suitable solubilizing agents are ethoxylated fatty acid sorbitan partial esters. Those are preferably addition products of 20 to 60 moles of ethylene oxide to sorbitan monoesters and sorbitan diesters of fatty acids with 12 to 18 C atoms. Equally suitable solubilizing agents are fatty acid partial esters of glycerol or sorbitan ethoxylates. Those are preferably mono- and di-esters of C₁₂-C₁₈ fatty acids and addition products of 20 to 60 moles of ethylene oxide per mole of glycerol or per mole of sorbitol.

The oral, dental and/or dental prosthesis care agents according to the invention comprise preferably as solubilizers for the aroma oils optionally contained, addition products of 20 to 60 moles ethylene oxide to hydrogenated or non-hydrogenated castor oil (i.e., to oxystearic acid or ricinoleic acid triglycerides), to glycerol mono- and/or di-stearate or to sorbitan mono- and/or di-stearate.

Other common additives for the oral, dental and/or dental prosthesis care agents are, for example, pigments, e.g., titanium dioxide, and/or coloring agents; pH-adjusting agents and buffer substances, such as sodium bicarbonate, sodium citrate, sodium benzoate, citric acid, phosphoric acid, or acid salts such as NaH₂PO₄; wound-healing and anti-inflammatory substances such as allantoin, urea, panthenol, azulene or chamomile extract; substances active against calculus, such as organophosphonates, e.g., hydroxyethane diphosphonate or azacycloheptane diphosphonate; preservatives such as sorbic acid salts and p-hydroxybenzoic acid esters; plaque inhibitors such as hexachlorophene, chlorhexidine, hexetidine, triclosan, bromchlorophen, and phenylsalicylic acid esters.

In one special embodiment the composition is a mouth rinse, a gargle, a denture cleaner, or a denture adhesive.

Aside from the ingredients already mentioned for oral, dental and/or dental prosthesis care agents, per-compounds such as peroxyborate, peroxymonosulfate or percarbonate are suitable for denture cleaners according to the invention, especially denture cleaning tablets and powders. They have the advantage that, aside from the bleaching action, they also have deodorizing and/or disinfectant actions. Such per-compounds are used in denture cleaners at from 0.01 to 10% by weight, especially from 0.5 to 5% by weight.

Enzymes, such as proteases and carbohydrases, to degrade proteins and carbohydrates, are also suitable ingredients. The pH can be between 4 and 12, especially between 5 and 11.

Still other additives are required for denture cleaner tablets, such as agents that produce a bubbling effect, such as substances that release CO₂, such as sodium bicarbonate; fillers, such as sodium sulfate or dextrose; lubricants such as magnesium stearate, flow-control agents such as colloidal silicon dioxide; and granulating agents, such as the previously mentioned high-molecular-weight polyethylene glycols or polyvinylpyrrolidone.

Denture adhesives can be offered as powders, creams, films or liquids. They promote adhesion of the prosthesis.

Natural and synthetic swelling agents are suitable ingredients. Natural swelling agents include alginates, plant gums such as gum Arabic, tragacanth and karaya gums, and natural rubber. In particular, alginates and synthetic swelling agents such as sodium carboxymethylcellulose, high-molecular-weight ethylene oxide copolymers, salts of poly(vinylether-co-malic acid) and polyacrylamide are suitable.

Hydrophobic principles, especially hydrocarbons such as white Vaseline (German Pharmacopeia) or paraffin oil are suitable auxiliary substances for liquid products and pastes.

In a preferred embodiment, the compositions according to the invention comprise at least one antiperspirant ingredient. Suitable antiperspirant ingredients according to the invention include water-soluble astringent or protein-coagulating metallic salts, especially organic and inorganic salts of aluminum, zirconium, zinc and titanium, as well as arbitrary mixtures of those salts. Water-solubility, according to the invention, is understood to be solubility of at least 4 g active substance per 100 g solution at 20° C. Usable according to the invention, for instance, are alum (KA1(SO₄)₂.12H₂O), aluminum sulfate, aluminum lactate, sodium aluminum chlorohydroxylactate, aluminum chlorohydroxyallantoinate, aluminum chloride hydrate, aluminum phenolsulfonate, aluminum zirconium chloride hydrate, zinc chloride, zinc phenolsulfonate, zinc sulfate, zirconium chloride hydrate, aluminum zirconium chloride hydrate-glycine complex and complexes of basic aluminum chlorides with propylene glycol or polyethylene glycol. The liquid preparations preferably comprise an astringent aluminum salt, especially aluminum chloride hydrate and/or an aluminum-zirconium compound. Aluminum chloride hydrates are marketed, for example, in the powder form as MICRO DRY® Ultrafine, or in activated form as REACH® 501 or REACH® 103 by Reheis, or in the form of aqueous solutions as LOCRON® L by Clariant or as CHLORHYDROL® by Reheis. Reheis offers an aluminum sesquichloride hydrate under the name REACH® 301. Use of aluminum-zirconium tri- or tetra-chlorohydrex-glycine complexes, commercially available from Reheis, for example, under the name REZAL®36G is also especially advantageous according to the invention.

The compositions according to the invention comprise the antiperspirant agent in a proportion of 0.10-40% by weight, preferably 2-30% by weight and especially 5-25% by weight, based in each case on the total composition.

In a further preferred embodiment the compositions according to the invention comprise at least one other deodorant ingredient. Fragrances, antimicrobial, antibacterial, microorganism-inhibiting substances, enzyme inhibitors, antioxidants and odor adsorbents are suitable as further deodorant ingredients according to the invention.

In particular, organohalogen compounds and halides, quaternary ammonium compounds and zinc compounds are suitable. Chlorhexidine and chlorhexidine gluconate, benzalkonium halides and cetylpyridinium chloride are preferred. Sodium bicarbonate, sodium phenolsulfonate and zinc phenolsulfonate, the components of linden flower oil, phenoxyethanol, Triclosan (IRGASAN® DP300) or triethyl citrate are also usable.

The preferred enzyme inhibitors are inhibitors of enzymes of the axillary microbial flora that are involved in production of body odor. They are preferably lipases, arylsulfatases (see WO 01/99376), β-glucuronidases (see WO 03/039505), 5-α-reductases and aminoacylases.

Other antibacterially active deodorant ingredients are lantibiotics, glycoglycerolipids, sphingolipids (ceramides), sterols and other ingredients that inhibit adhesion of bacterial to the skin, such as glycosidases, lipases, proteases, carbohydrates, di- and oligo-saccharide fatty acid esters, and alkylated mono- and oligo-saccharides.

Other suitable deodorant ingredients are water-soluble polyols, selected from water-soluble diols, triols, and higher-functional alcohols as well as polyethylene glycols. Of the diols, C₂-C₁₂ diols are suitable, especially 1,2 propylene glycol; butylene glycols such as 1,3-butylene glycol, 1,3-butylene glycol and 1,4-butylene glycol; pentanediols such as 1,2-pentendeiol, and hexanediols, such as 1,6-hexanediol. Glycerol and industrial oligoglycerol mixtures with a self-condensation degree of 1.5 to 10, such as industrial diglycerol mixtures with a diglycerol content of 40 to 50% by weight or triglycerol are also suitable, as are 1,2,6-hexanetrol and polyethylene glycols (PEG) with an average molecular weight of 100 to 1,000 Dalton, such as PEG-400, PEG-600, or PEG-1000. Other suitable higher-functional alcohols are the C₄, C₅ and C₆ monosaccharides and the corresponding sugar alcohols, e.g., mannitol or sorbitol.

Deodorant or antiperspirant sticks can be in gelled form, based on anhydrous wax and based on W/O and O/W emulsions. Gel sticks can be produced on the basis of fatty acid soaps, dibenzylidenesorbitol, N-acylamino acid amides, 12-hydroxysteaeric acid and other gel-formers. Aerosol sprays, pump sprays, roll-on applicators and creams can be water-in-oil emulsions, oil-in-water emulsions, silicone oil-in-water emulsions, water-in-oil microemulsions, oil-in-water microemulsions, silicone oil in water microemulsions, anhydrous suspensions, alcoholic and hydroalcoholic solution, aqueous gel, or oils. All the compositions mentioned can be thickened, for example, on the basis of fatty acid soaps, dibenzylidenesorbitol, N-acylamino acid amides, 12-hydroxystearic acid, polyacrylates of the Carbomer and Carbopol type, polyacrylamides and polysaccharides, which can be chemically and/or physically modified. The emulsions and microemulsions can be transparent, translucent or opaque.

Liquid and gel administration forms of the compositions according to the invention can comprise thickeners, e.g., cellulose ethers, such as hydroxypropylcellulose, hydroxyethylcellulose and methylhydroxypropyl-cellulose, thickening polymers based on polyacrylates, which may optionally be cross-linked, such as the Carbopol types or PEMULEN® products, or based on polyacrylamides or polyacrylates containing sulfonic acid groups, such as SEPIGEL® 305 or SIMULGEL® EG, and also inorganic thickeners, e.g., bentonite and hectorite (LAPONITE®).

The compositions according to the invention can comprise other substances with cosmetic and dermatologic action, such as anti-inflammatory substances, solids selected from silicic acids, e.g., AEROSIL® types, silica gels, silicon dioxide, clays, e.g., bentonite or kaolin, magnesium aluminum silicates, e.g., talc, boron nitride, titanium dioxide, which can optionally be coated, optionally modified starches and starch derivatives, cellulose powders and polymer powders, furthermore plant extracts, protein hydrolyzates, vitamins, perfume oils, sebostats, anti-acne agents and keratolytics.

The compositions according to the invention, if they are liquid, can be applied to flexible and absorbent carriers and offered as deodorant or antiperspirant towels or sponges. Suitable flexible and absorbent carriers in the sense of the invention are, for example, carriers of textile fibers, collagen or polymeric foam materials. Both natural fibers such as cellulose (cotton, linen), silk, wool, regenerated cellulose (viscose, rayon), or cellulose derivatives, and synthetic fibers such as polyester, polyacrylonitrile, polyamide or polyolefin fibers or mixtures of such fibers, woven or nonwoven, can be used as textile fibers. These fibers can be processed into absorbent cotton pads, nonwoven fabrics, or cloths or knitted fabrics. Flexible and absorbent polymeric foam materials, e.g., polyurethane foams and polyamide foams, are also suitable substrates. The substrate can have one, two, three, or more than three layers, and the individual layers can be of the same or different materials. Each substrate layer can have a homogeneous or inhomogeneous structure with, for instance, different zones of different density.

In the sense of the invention, those carrier substrates are considered absorbent which can bind at least 10% by weight water at 20° C. by adsorption or capillarity; but carriers that can bind at least 100% by weight water by adsorption or capillarity are preferred.

The carrier substrate can be produced by treating the absorbent flexible carrier substrate, preferably of textile fibers, collagen or polymeric foam materials, with the compositions according to the invention and optionally drying it. The treatment of the carrier substrate can be by any desired process, e.g., by spraying, immersion and squeezing, soaking, or simply by injecting the composition according to the invention into the carrier substrate.

The form of administration as an aerosol is further preferred according to the invention. In that case the cosmetic preparation comprises a propellant, selected from propane, butane, isobutene, pentane, isopentane, dimethyl ether, hydrofluorocarbons and chlorofluorocarbons. Likewise, a compressed propellant such as air, nitrogen or carbon dioxide can be used. Mixtures of the propellants named can likewise be used.

In a preferred embodiment, the compositions according to the invention are in the form of a liquid or solid oil-in-water emulsion, water-in-oil emulsion, multiple emulsion, microemulsion, PIT emulsion or Pickering emulsion, a hydrogel, a lipogel, a single-phase or multiphase solution, a foam, a powder, or a mixture with at least one polymer suitable as a medicinal adhesive. The agent can also be administered in anhydrous form, such as an oil or a balm. In this case the carrier can be a vegetable or animal oil, a mineral oil, or a mixture of such oils.

In one special embodiment of the agent according to the invention the agent is a microemulsion. Within the limits of the invention, microemulsions are understood to be not only the thermodynamically stable microemulsions but also the so-called “PIT” emulsions. Those emulsions are systems of the 3 components, water, oil, and emulsifier, which exist as an oil-in-water emulsion at room temperature. On warming, these systems form microemulsions in a certain temperature range (called the phase-inversion temperature or “PIT”). On further warming they change into water-in-oil (W/O) emulsions. On subsequent cooling, O/W emulsions are formed again, but at room temperature they exist as microemulsions or as very finely divided emulsions with an average particle diameter less than 400 nm and particularly about 100-300 nm. Those microemulsions or “PIT” emulsions having an average particle diameter of about 200 nm can be preferred according to the invention. For details about these “PIT emulsions” see, for example, the printed article in Angew. Chem. 97, 655-669 (1985).

According to a preferred embodiment of the present invention, the composition comprises, along with β-defensin 2 and/or its derivatives, especially human β-defensin 2, also at least one sebum regulator for unclean skin or for treatment of acne, especially mild acne. Furthermore the preferred combination can also be used as a hair care agent, especially a hair shampoo. These hair care agents have the advantage that there is a synergistic effect between the antimicrobially active β-defensin 2 and the sebum regulator, so that, preferably, fatty hairs and/or dandruff can be treated successfully. The keratinophylic fungus Malassezia is considered the cause of increased flaking of the skin, on the head, for example (dandruff).

Especially suitable sebum-regulatory ingredients are: azelaic acid, sebacic acid, 10-hydroxydecanoic acid, 1,10-decanediol (the ternary combination of sebacic acid, 10-hydroxydecanoic acid and 1,10 decanol, such as Acnacidol PG (Vicience), Azeloglicina (potassium azeloyl diglycinate, Sinerga), extracts from Spiraea ulmaria (such as contained, for instance, in the product Seboregul from the company Silab) is particularly preferred.) Further preferred are water-soluble and oil-soluble extracts from witch hazel, burdock root and nettle, cinnamon tree extract (e.g., SEPICONTROL® A5 from Seppic, chrysanthemum extract (e.g., LARICYL® from Laboratoires Sérobiologiques), ASEBIOL® (Laboratoires Sérobiologiques), INCI: water, hydrolyzed yeast protein, pyridoxine, niacinamide, glycerol, panthenol, bioton), ANTIFETTFAKTOR® CIS-218/2-A (Cosmetochem, INCI: water, cetyl-PCA, PEG-8 isolauryl thioether, PCA, cetyl alcohol).

According to a further preferred embodiment of the present invention, the composition according to the invention comprises, in addition to β-defensin 2 and/or its derivatives, especially human β-defensin 2, at least one anti-inflammatory and/or skin-soothing ingredient.

Especially suitable anti-inflammatory ingredients are selected from: Silymarin Phytosome (INCI: Silybum Marianum Extract and Phospholipids) from Indena SpA; extracts of Centella asiatica, obtainable, for instance, under the name Madecassicoside from DSM; glyccyrrhizin, particularly preferably encapsulated in liposomes and available in that form, for example, under the trade name Calmsphere from Soliance. Mixtures of cereal waxes, extracts of shea butter and Argania spinosa oil with the INCI designation “Spent grain wax and Butyrospermum parkii (shea butter) extract and Argania spinosa kernel oil, such as is obtainable, for instance, under the trade name Stimu-Tex AS from the company Pentapharm. Extracts of Vanilla tahitensis, such as are available, for example, under the trade name Vanirea (INCI: Vanilla tahitensis Fruit Extract) from Solabia. Extracts of olive leaves (INCI: Olea europaea (Olive) Leaf Extract), as is obtainable particularly under the trade name Oleanoline DPG from Vincience. Also algin hydrolyzates, such as are available under the trade name Phycosaccharides, especially Phycosaccharide AI, from Codif. Extracts of Bacopa Monniera, such as are available, for example, under the trade name Bacocalmine from Sederma, extracts from the rooibos plant, such as are available under the trade name Rooibos Herbasec MPE from Cosmetochem; the physiologically compatible salts of sterol sulfates, such as are obtainable under the trade name Phytocohesine (INCI: Sodium beta-sitosteryl sulfate) from Vincience; α-bisabolol, α-lipoic acid, panthenol, or Fucogel 100 (Solabia).

Use of β-defensin 2 and/or its derivatives, especially human β defensin 2, in combination with substances having anti-inflammatory activity, makes possible a particularly gentle non-irritating treatment of sensitive skin, dry skin, atopic dermatitis, old skin, UV-damaged skin and/or irritated skin. The combination of the two substances causes both immediate alleviation of the complaints because of the anti-inflammatory ingredient and a lasting improvement through the use of β-defensin 2.

A particularly preferred embodiment of the present invention is a composition comprising β-defensin 2 and/or its derivatives, especially human β defensin 2, and at least one substance with prebiotic activity.

It was found, surprisingly, that the combination of β-defensin 2 and/or its derivatives, especially human β-defensin 2, with substances having prebiotic activity exerts a synergistic effect on the health of the skin or on the development of body odor. The combination of β-defensin 2 and substances with prebiotic activity leads to stabilization of the skin microflora, especially of the ratio of Propionibacterium acnes to Staphylococcus epidermidis and Bacillus lichenformis.

A further object of the present invention is use of β-defensin 2 and/or its derivatives, especially human β-defensin 2 particularly on the skin, especially in the axillary region and on the facial skin, as well as for oral, dental and denture care, in combination with prebiotically active substances, especially plant extracts, glycerol monoalkyl ethers or esters of organic acids to inhibit the grown and/or the physiological activity of undesirable microorganisms.

A further object of the present invention is use of compositions comprising β-defensin 2 and/or its derivatives, especially human β-defensin 2, and prebiotically active substances, principally on the skin, especially substances prebiotically active in the axillary region, especially plant extracts, glycerol monoalkyl ethers or esters of organic acids to promote the growth and/or the physiological activity of desirable skin microorganisms, the desired skin microorganisms being preferably beneficial and/or non-pathogenic and/or skin-friendly and/or saprophytic skin microorganisms and/or, and especially preferred, odor-neutral microorganisms, especially odor-neutral coagulase-negative Staphylococci, principally S. epidermidis.

Those substances are particularly preferred that shift the microfloral profile of strongly-smelling subjects toward the microfloral profile of weakly smelling subjects and/or that seek substances that selectively promote the growth and/or the physiological activity of odor-neutral microorganisms, especially odor-neutral Staphylococci, principally of S. epidermidis and/or simultaneously inhibit the growth and/or the physiological activity of odor-producing Staphylococci, especially of S. hominis, and/or of Gram-positive anaerobic cocci, especially of Streptococci, principally of Anaerococcus octavius, and/or of odor-producing Corynebacteria and/or odor-producing micrococci, principally Micrococcus luteus.

In a particularly preferred embodiment, those substances or microorganisms are preferred that inhibit the growth and/or the physiological activity of S. hominis and at the same time to not affect, or promote the growth and/or the physiological activity of S. epidermidis.

The Gram-positive anaerobic cocci are preferably, according to the invention, the genus Peptostroptococcus. The generic designation Peptostreptococcus includes the genus synonyms Peptoniphilus, Gallicola, Slackia, Anaerococcus (including Anaerococcus octavius), Finegoldia, Micomonas, Atopobium and Ruminococcus. Thus the Gram-positive cocci that participate in development of body odor, against which the substances according to the invention are effective, are selected from bacteria of these genera in a preferred embodiment.

Prebiotic activity is understood, according to the invention, to mean that the growth and/or the physiological activity of the desired, especially skin-friendly and/or odor-neutral skin microorganisms or microflora are promoted over the growth and/or the survival ability of the undesired skin microorganisms or microflora, especially the skin microorganisms or microflora that are harmful to the skin and/or odor-forming. That can be achieved both by the active ingredient promoting the growth of the desired skin microorganisms without a direct effect on the growth of the undesired skin microorganisms, and also by the active ingredient inhibiting the growth of the undesired skin microorganisms without a direct effect on the growth of the desired skin microorganisms. In an embodiment that is particularly preferred according to the invention and especially surprising, though, the active ingredient promotes the growth of the desired skin microorganisms and simultaneously inhibits growth of the undesired skin microorganisms.

The undesired microorganisms here can in particular be the microorganisms that are harmful to the skin, and/or pathogenic microorganisms and/or microorganisms that grow to excessive densities and so may exert an undesired and/or pathogenic effect. The undesired microorganisms can also be microorganisms that produce odor or cause an unpleasant smell.

The desired microflora here involve, correspondingly, microorganisms that are good for the skin and/or non-pathogenic microorganisms, especially the resident skin flora, saprophytic microorganisms or, in the case of body odor, microorganisms that are odor-neutral; that is, which to not produce bad-smelling compounds from the components of sweat or from other substances. It must be taken into special consideration here that promotion of the growth of the desired microorganisms suppresses the undesired microorganisms and, conversely, inhibition of the growth of the undesired microorganisms promotes the growth of the desired ones, so that the prebiotic effect can be brought about in different ways.

In the case of (unpleasant) body odor, and especially armpit odor, the undesired microorganisms are not necessarily pathogenic microorganisms. Instead, the odor-forming microorganisms can be natural ones that are themselves good for the skin. In the case of (unpleasant) body odor, then, the undesired microorganisms are defined as those that cause body odor. In this connection, a prebiotic substance is distinguished in that it promotes growth of the odor-neutral microorganisms at the cost of the growth of the odor-forming microorganisms (which cause unpleasant body odor).

The terms “odor-forming microorganisms” or “odor microorganisms” are understood, according to the invention, to be those microorganisms that occur more in humans with body odor. They are preferably microorganisms that either themselves produce substances, or which promote the formation of substances, that cause an unpleasant odor. Furthermore, they can also be microorganisms that are only indirectly involved in formation of such substances, for example, in that they produce a substance or promote the formation of substances that can be converted by other microorganisms into substances with unpleasant odor. Therefore, according to the invention, the odor-producing microorganisms do not necessarily have to be those that cause the unpleasant odor themselves, but they can also be involved in other ways in the metabolism of odor production.

A further object of the present invention is a cosmetic or pharmaceutical composition containing β-defensin 2 and/or its derivatives, especially human β-defensin 2, and a prebiotically active substance, principally a plant extract having a prebiotic action on the skin, a glycerol monoalkyl ether having a prebiotic action on the skin, an ester of an organic acid having a prebiotic effect on the skin, or mixtures of them, with the cosmetic or pharmaceutical composition being preferably a topical skin treatment agent, especially for the face, mouth or axillary region, especially a deodorant and/or antiperspirant or an oral, dental, or dental prosthesis care agent.

In particular, the undesired microorganisms are preferably microorganisms that are harmful to the skin and/or pathogenic microorganisms, especially preferably coagulase-positive Staphylococci, particularly S. aureus; or Gram-negative bacteria, preferably Pseudomonads, especially P. aeruginosa and/or, and particularly preferably, odor-producing microorganisms, particularly odor-producing Staphylococci, principally S. hominis; odor-producing Gram-positive anaerobic cocci, especially Peptostreptococci, principally Anaerococcus octavius, and/or odor-producing Cornyebacteria and/or odor-producing Micrococci, principally Micrococcus luteus.

The undesirable microorganisms in the oral region are preferably cariogenic bacteria, especially preferably Streptococci, particularly Streptococcus mutans, S. salivarius and S. mitis, Gram-negative pathogens causing gingivitis, especially Porphyromonas gingivalis, Treponema denticola, Fusobacterium nucleatum and Actinomyces naeslundii.

Use in this case can be principally in topical cosmetic skin treatment agents and/or oral, dental, and dental prosthesis care agents.

According to the invention the concept “skin” is understood to mean preferably the skin itself, especially human skin, but also the mucous membrane and structures within the skin, if they involve living cells, particularly hair follicles, hair roots, hair bulbs, the ventral epithelium of the nail bed (Lectulus) as well as sebaceous glands and sweat glands. In a preferred embodiment, ‘skin’ is understood according to the invention to be the facial skin and/or the armpit skin (axillary region).

The composition according to the invention is preferably suited for shifting the microfloral profile of humans with strong or unpleasant body odor to the microfloral profile that appears for humans without body odor, or is able to reproduce and/or stabilize such a microfloral profile.

Thus a further object of the present invention is a composition comprising β-defensin 2 and/or its derivatives, especially human β-defensin 2 and a substance with prebiotic action, which acts to inhibit odor in the armpit region, preferably in that it promotes growth of odor-neutral Staphylococci, especially S. epidermidis, and/or inhibits the growth of odor-producing Staphylococci, especially S. hominis, and/or inhibits the growth of Gram-positive anaerobic cocci, especially Anaerococcus octavius and/or inhibits the growth of odor-producing Corynebacteria and/or odor-producing Micrococci, especially Micrococcus luteus.

In a preferred embodiment of the invention the substance with prebiotic activity is a plant extract, a glycerol monoalkyl ether or an ester of an organic acid which promotes the growth of odor-neutral coagulase-negative Staphylococci, especially S. epidermidis and at the same time exhibits an inhibitory or no direct effect on the growth of odor-producing Staphylococci, especially S. hominis.

In a further preferred embodiment of the invention the substance with prebiotic activity is a plant extract, a glycerol monoalkyl ether or an ester of an organic acid which inhibits the growth of odor-producing Staphylococci, especially S. hominis and at the same time exhibits an promoting effect or no direct effect on the growth of odor-neutral Staphylococci, especially S. epidermidis.

A further object of the present invention is use of the prebiotically active substances, especially those prebiotically active on the skin, in topical cosmetic skin treatment agents for treatment of body odor, especially in the armpit region, principally by use in deodorants and/or antiperspirants.

A further object of the present invention is use of β-defensin 2 and/or its derivatives, especially human β-defensin 2 in combination with prebiotically active substances, especially those prebiotically active on the skin, in topical cosmetic skin treatment agents to treat unclean, dry, or oily skin and for treatment of skin fungi or dandruff.

Thus a further object of the present invention is use of compositions containing β-defensin 2 and/or its derivatives, especially human β-defensin 2, in combination with prebiotically active substances, especially those prebiotically active on the skin, especially plant extracts, in topical cosmetic skin treatment agents to inhibit growth of undesired skin microorganisms, with the undesired skin microorganisms being preferably microorganisms that are bad for the skin and/or pathogenic microorganisms and/or coagulase-positive Staphylococci, especially S. aureus, or microorganisms selected from the group consisting of Propionibacterium acnes, Candida albicans, Malassezia furfur, Corynebacterium spp. or Peptostreptococcus spp., principally Propionibacterium acnes.

In a specially preferred embodiment according to the invention the plant extract with prebiotic activity is an extract that acts to promote growth of coagulase-negative Staphylococci, especially S. epidermidis or S. Warneri and simultaneously has an inhibitory effect on growth of Propionibacterium acnes.

Preferably the compositions according to the invention is suited to reproduce or stabilize the naturally occurring healthy microbial equilibrium of the skin flora. In this situation the treatment can also be preventive or prophylactic.

In this situation the prebiotically active substance is preferably contained in the composition in a proportion of 0.01 to 20, especially preferably 0.05 to 10, principally 0.1 to 5, particularly 0.1 to 1.5 or 0.5 to 2% by weight, based in each case on the total composition.

The prebiotically active plant extract according to the invention is preferably a tea extract, especially from the family of the Theaceae or the family of the Malvaceae, an extract from the family of the Vitaceae, from the family of the Apiaceae, the Buxaceae, the Zingiberales or an extract from the family of the Asteraceae or mixtures of those. Especially preferably, it is an extract from the family of the Vitaceae.

The extract from the family of the Theaceae is preferably an extract from Camellia species, principally an extract from white tea (Camellia sinensis). In a preferred embodiment here it is an extract from the leaves, such as is obtainable from Cosmetochem, for example.

The extract from the family of the Malvaceae is preferably an extract of Hibiscus species, principally an extract from Sudan tea (karkade, hibiscus, Hibiscus sabdariffa), or an extract from Malva species, principally an extract of mallow (Malve sylvestris), especially of the mallow flowers.

The extract from the family of the Vitacea is preferably an extract of Vitis species, principally an extract of wine grapes (Vitis viticola). In this case it is, particularly preferably, and extract of wine grape seeds.

The extract from the family of the Apiaceae is preferably an extract of Daucus species, principally of carrot (Daucus carota), or an extract of Commiphora species, principally of myrrh (Commiphora myrrha). Here, in a preferred embodiment, it is an extract from the roots, such as is available, for example, from Cosmetochem or Rahn.

The extract from the family of the Buxaceae is preferably an extract of Simmondsia species, principally of Jojoba (Simmondsia chinensis).

The extract from the family of the Asteraceae is preferably an extract of Calendula species, principally of the marigold (Calendula officinalis).

The prebiotically active plant extract according to the invention is preferably an extract from a conifer, especially from the group of the Pinaceae, or an extract from the group of the Sapindaceae, Araliaceae, Lamiaceae or Saxifragaceae, or mixtures of those.

The plant extract is especially preferably an extract of Picea spp., particularly an extract of Picea excelsa (synonym Picea abies, spruce) or of Picea glauca (sugarloaf spruce, Norway spruce), Paullinia sp. (Guarana), Panax sp. (ginseng), Lamium album (white nettle) or Ribes nigrum (black currant) or mixtures of them.

In a preferred embodiment according to the invention the extract is from Sapindaceae, and particularly of Guarana, a dry extract from seeds.

The extract from conifers, and especially from Pinaceae is preferably, according to the invention, an extract from the needles. The extract of Picea abies or Picea excelsa is preferably here a water/propylene glycol extract, and the extract of P. glauca is a water/ethanol extract. The extract of Araliaceae, particularly of ginseng, is preferably a root extract. The extract of Lamiqaceae, particularly of white nettle, is preferably a water/propylene glycol extract. The extract of Saxifragaceae, particularly of black current, is preferably a water/propylene glycol extract. The extract of the Zingiberales order is preferably an extract of the family of the Zingiberaceae, particularly from Curcuma sp., principally from Curcuma zedoaria. The plant extracts named above are obtainable, for example, from the companies Cosmetochem (Germany) or Rahn (Germany).

A quite particularly preferred embodiment involves the following plant extracts: 1. Seed extract of grapes (Vitis viticola) (Cosmetochem; water/propylene glycol extract); 2. Leaf extract from white tea (Camellia sinensis) (Cosmetochem; water/ethanol dry extract); 3. Extract of karkade (hibiscus, Sudan tea, Hibiscus sabdariffa) (Cosmetochem; water/ethanol dry extract); 4. Flower extract from mallow (Malve sylvestris) (Cosmetochem; water/ethanol dry extract); 5. Extract of wine grapes (Vitis viticola), (Cosmetochem; water/propylene glycol extract) 6. Mixed extract of carrot and jojoba (Daucus carota and Simmondsia chinensis), (Flavex, CO2 extract); 7. Extract of myrrh (Commiphora myrrha), (Cosmetochem; water/propylene glycol extract); 8. Extract of marigold flower (Calendula officinalis), (Cosmetochem, water/ethanol dry extract); 9. Curcuma; 10. Norway spruce; 11. Bambusa vulgaris; 12. Ginseng; 13. Wild rose hips (Rosa canina); 14. Epica (Ribes nigrum and Pinus sylvestris).

The prebiotically active plant extract can be produced essentially in any of the ways known to those skilled in the art, using any desired plant tissue and using any desired extractant. Thus the plant extract can be obtained, for example, by extracting the whole plant, extracting the flowers, leaves, seeds, roots, and/or by extracting the meristem of the plant.

Water, alcohols, and their mixtures can be used, for example, as extractants to produce the plant extracts named. The alcohols to be considered include, for example, lower alcohols such as ethanol and isopropanol, but especially also multifunctional alcohols such as ethylene glycol, propylene glycol and butylene glycol, either as the sole extractant or in mixtures with water. For instance, plant extracts based on water/propylene glycol in proportions of 1:10 to 10:1 have proven to be especially suitable. The extraction can, for example, be done in the form of steam distillation. A dry extraction may optionally also be done.

The extract of Theaceae, Malvaceae and Asteraceae is preferably a water/propylene glycol extract or a water/ethanol dry extract or a water/ethanol extract on maltodextrin carriers. The extract of Vitaceae is preferably a water/propylene glycol extract. The extract of Apiaceae is preferably a CO₂ extract or a water/propylene glycol extract. The extract of Buxaceae is preferably a CO₂ extract.

In a preferred embodiment of the present invention, the selection of the extract is determined by the composition in which the extract is to be used. For instance, aqueous extracts, especially water/propylene glycol extracts are preferably used in aqueous or alcoholic compositions or soap-containing sticks. Oil-soluble extracts are preferably used in compositions that contain oil, especially in antiperspirant sticks or antiperspirant aerosols. Extracts on maltodextrin carriers can be used in both hydrophilic and hydrophobic products.

The plant extracts that are prebiotically active on the skin can be used, according to the invention, both in pure and in dilute form. When they are used in dilute form, they usually contain about 2-80% by weight active substance with the extractant or mixture of extractants used to obtain them as the solvent. Depending on the choice of extractant, it may be preferable to stabilize the plant extract by adding a solubilizer. Examples of suitable solubilizers are ethoxylation products of optionally hydrogenated animal and vegetable oils. Preferred solubilizers are ethoxylated mono, di, and tri-glycerides of C₈₋₂₂ fatty acids having 4 to 50 ethylene oxide units, such as ethoxylated castor oil, olive oil ethoxylate, almond oil ethoxylate, mink oil ethoxylate, polyoxyethylene glycol caproic/caprylic acid glyceride, polyoxyethylene glycerol monolaurate and polyoxyethylene glycol coco fatty acid glyceride.

The prebiotically active glycerol monoalkyl ether is preferably a 1 alkyl glycerol ether. Here the alkyl group is preferably a (C₂-C₁₄), especially a (C₄-C₁₂), and principally a (C₆-C₁₀) alkyl group, in which the alkyl group can be either straight-chain or branched. In a particularly preferred embodiment, the alkyl group is a branched octyl group and/or an alkylhexyl group, especially an ethylhexyl group, principally a 2-ethylhexyl group. 1-(2-ethylhexyl) glyceryl ether is available, for example, under the trade name of Sensiva® SC 50 (Schülke & Mayr, Germany).

The prebiotically active ester of an organic acid is preferably an ester of a (C₁₀-C₁₈) carboxylic acid with a (C₁-C₁₀) alcohol, in which both the carboxylic acid group and the alcohol group can be linear or branched and saturated or unsaturated, and in which the alkyl groups of the carboxylic acid and alcohol groups can bear one or more substituents independently of each other, especially selected from (C₁-C₆)-alkyl and hydroxy. The carboxylic acid is especially preferably a C₁₂-C₁₆)-carboxylic acid, principally a C₁₋₄-carboxlic acid, especially myristic acid. The alcohol is particularly preferably a (C₁-C₆)-alkanol, principally methanol, ethanol, propanol, particularly 1 propanol, 2-propanol or isopropanol, butanol, particularly 1-butanol, 2-butanol, or tert.-butanol, pentanol, particularly 1-pentanol, 2-pentanol, 3 pentanol, 2-methyl-1-butanol, 3-methyl-1-butanol, 2-methyl-2-butanol or 3-methyl-2-butanol. In a particularly preferred embodiment the prebiotically active ester is isopropyl myristate or ethyl myristate.

The composition according to the invention is particularly suited for use as a skin and/or hair care agent, pharmaceutical compositions or deodorant. According to a further especially preferred embodiment the composition comprises at least one deodorizing ingredient and/or one antiperspirant. The positive effect of the combinations according to the invention on the equilibrium of the microflora in the armpit can be improved even more by addition of deodorizing ingredients and/or antiperspirants.

A further object of the invention is use of β-defensin 2 and/or its derivatives, especially human β-defensin 2, as an antimicrobial agent in washing or cleaning agents, in cosmetics or pharmaceuticals, in agents for water treatment, especially in closed water loops and in cooling lubricants.

A further object of the present invention is use of β-defensin 2 and/or its derivatives, especially human β-defensin 2, especially in sterilizing, disinfecting, washing, dishwashing and cleaning agents, principally in cleaners to remove, kill and/or inhibit microorganisms and/or to remove organic contamination from hard surfaces. This use can be in the household or in the area of medications, foods, brewing, medical technology, dyeing, wood, textile, cosmetic, leather, tobacco, fur, rope, paper, cellulose, plastics, fuel, oil, rubber or machine industries or in dairies. There the compositions used according to the invention can be used particularly to remove, kill and/or inhibit microorganisms in or on household and/or hygienic items as well as in cellulose and paper mills, in circulating cooling towers, and in other systems conducting flowing and/or circulating water.

Thus further objects of the present invention are sterilizing, disinfecting, washing, dishwashing, and cleaning agents which comprise according to the invention β-defensin 2 and/or its derivatives, especially human β-defensin 2.

Thus the use according to the invention is preferably in sterilizing, disinfecting, impregnating agents or preservatives, washing or cleaning agents, or in coolants or in cooling lubricants (technological application solutions), and in the area of water purification/water treatment as well as in the area of medications, foods, brewing, medical technology, dyeing, wood, textile, cosmetic, leather, tobacco, fur, rope, paper, cellulose, plastics, fuel, oil, rubber or machine industries or in dairies.

The agent can fundamentally be an agent for any desired kind of surface. Therefore the surface may be biotic or abiotic, artificially synthesized or natural, soft or hard. The surface can be a textile, ceramic, metal and/or plastic surface. The item can, for example, be washing, dishes, hygienic equipment, floor coverings, shoes, leather, useful objects made of rubber, ship hulls, prostheses, teeth, tooth replacements or catheters.

The cleaning agent can in particular be a household cleaner or a cleaner for industrial plants, especially the former. In a preferred embodiment the cleaner is one for cleaning hard surfaces, such as floors, glazed tiles, dishes, wall tiles, plastics and other hard surfaces in the household, in industrial plants, in public hygienic systems, in swimming pools, saunas, sports grounds, or in medical or massage practices.

According to a further preferred embodiment of the present invention, the composition is especially suitable as a cleaning agent for hard surfaces, especially bathrooms and restrooms.

The cleaning agent can contain the active ingredient according to the invention preferably in proportions of 0.005 to 10.0% by weight, preferably 0.02 to 0.2% by weight. The preferred final concentration according to the invention can then be obtained by diluting the cleaning agent. Here the cleaning agent can optionally comprise additional biocides, but it can also be biocide-free.

A liquid, gelled or paste aqueous cleaning agent can according to the invention contain, aside from the composition, also the usual ingredients of cleaning agents. Those ingredients include, for example, surfactants, builders, acids, alkalies, hydrotropes, solubilizers, thickeners, abrasives, as well as other auxiliary substances and additives such as coloring agents, perfumes, corrosion inhibitors or even skin care agents.

The optionally used surfactants are preferably selected from the group of anionic surfactants, nonionic surfactants and/or amphoteric surfactants. Use of anionic and/or nonionic surfactants is preferred. If they are used, anionic surfactants are used preferably in proportions of 0.1 to 15% by weight; nonionic surfactants preferably in proportions of 0.1 to 10% by weight; and amphoteric surfactants preferably in proportions of 0.1 to 4% by weight, based in each case on the total composition. In a less preferred embodiment, cationic surfactants can also be used in proportions of up to 2% by weight, based in each case on the total composition. Likewise, cationic surfactants can also be used. In a preferred embodiment, though, the cleaning agent is free of cationic surfactants because of their outgoing biocidal action.

The anionic surfactants usable according to the invention include aliphatic sulfates such as fatty alcohol sulfates, fatty alcohol ether sulfates, dialkyl ether sulfates, monoglyceride sulfates, and aliphatic sulfonates such as alkane sulfonates, α-olefin sulfonates, ether sulfonates, n-alkylether sulfonates, sulfonated fatty acids, ester sulfonates and lignin sulfonates. Also usable in the bounds of the present invention are alkylbenzene sulfonates, fatty acid salts (soaps), fatty acid cyanamides, sulfosuccinates (sulfosuccinic acid monoalkyl esters and dialkyl esters), sulfosuccinamates, sulfosuccinamides, carboxylic acid amide ether sulfates, alkylpolyglycol ether carboxylates, fatty acid isethionates, acylaminoalkane sulfonates (fatty acid taurides, n-acyltaurides), fatty acid sarcosinates, ether carboxylic acids and alkyl(ether)phosphates and α-sulfofatty acid salts, acyl glutamates, monoglyceride disulfates and alkyl ethers of glycerol disulfate, and, finally, mixtures of them.

In the present invention, fatty acids or fatty alcohols or their derivatives, if not otherwise specified, stand for branched or unbranched carboxylic acids or alcohols or their derivatives with preferably 6 to 22 carbon atoms, especially 8 to 20 carbon atoms, especially preferably 10 to 18 carbon atoms, and extremely preferably 12 to 16 carbon atoms, for example, 12 to 14 carbon atoms. The fatty acids/alcohols or their derivatives with even numbers of carbon atoms are especially preferred for ecological reasons, particularly because of their plant origin as renewable raw materials, but the teaching of the invention is not limited to them. In particular, the ketoalcohols that can be obtained by the Roelen Oxo synthesis, with preferably 7 to 19 carbon atoms, especially 9 to 19 carbon atoms, especially preferably 9 to 17 carbon atoms, extremely preferably 11 to 15 carbon atoms, for example, 9 to 11, 12 to 15 or 13 to 15 carbon atoms are correspondingly usable according to the invention.

They are used in the form of their alkali metal and alkaline earth metal salts, especially the sodium, potassium and magnesium salts, as well as ammonium and mono, di, tri, or tetra-alkylammonium salts and, in the case of the sulfonates, also in the form of their corresponding acids, e.g., dodecylbenzenesulfonic acid. Because of their production, the alkyl ether sulfates always also contain residual amounts of non-alkoxylated fatty alcohol sulfates, particularly at low degrees of ethoxylation. Furthermore, the means according to the invention can also comprise soaps, i.e., alkali or ammonium salts of saturated or unsaturated C₆-C₂₂ fatty acids.

The anionic surfactants are preferably selected from the group comprising fatty alcohol sulfates in proportions of up to 5% by weight, alkylbenzene sulfonates in proportions of up to 7.5% by weight and soaps in proportions of up to 2% by weight, based in each case on the total composition, and their mixtures.

Suitable nonionic surfactants are, for example, C₈-C₁₈-alkylalcohol polyglycol ethers, alkyl polyglycosides as well as nitrogen-containing surfactants or their mixtures, especially of the first two. C₈-C₁₈-alkylalcohol polypropylene glycol/polyethylene glycol ethers can be described by the formula
R¹O—(CH₂CH(CH₃)O)ₚ(CH₂CH₂O)ₑ—H
in which R¹ stands for a linear or branched aliphatic alkyl and/or alkenyl group with 8 to 18 carbon atoms, p for 0 or numbers from 1 to 3, and e for numbers from 1 to 20. They are obtained by adding propylene oxide and/or ethylene oxide to alkylalcohols, preferably to fatty alcohols. It is also possible to use end-capped C₈-C₁₈-alkylalcohol polyglycol ethers, i.e., alkylalcohol polyalkyleneglycol ethers according to the formula above, in which the free —OH group is etherified.

Cleaners according to the invention can contain alkylalcohol polyglycol ethers in proportions of 0.1 to 4% by weight, based on the total composition group.

Further preferred nonionic surfactants are alkyl polyglycosides (APG) having the formula
R²O[G]ₓ,
in which R² stands for a linear or branched, saturated or unsaturated alkyl group with 8 to 22 carbon atoms, [G] stands for a glycosidically linked sugar group and x stands for a number from 1 to 10. The index number x indicates the degree of polymerization (DP degree); that is, the distribution of monoglycosides and oligoglycosides. Although x is always integral in a specific compound, and here can take on principally the values x=1 to 6, the value of x is an analytically determined mathematical quantity that is usually a fractional number. It is preferable to use alkylglycosides with an average degree of oligomerization, x, of 1.1 to 3.0. From the viewpoint of applications technology it is preferable to use those alkylglycosides with a degree of oligomerization less than 1.7 and in particular between 1.2 and 1.6. Xylose is used preferably as the glycosidic sugar, but glucose is also used in particular, the alkyl or alkenyl group R² can be derived from primary alcohols having 8 to 18, preferably 8 to 14, carbon atoms. Typical examples are caproic [hexyl] alcohol, caprylic [octyl] alcohol, capric [decyl] alcohol and undecyl alcohol as well as their industrial mixtures, such as occur for example in the course of hydrogenation of industrial fatty acid methyl esters or in the course of hydrogenation of aldehydes from the Roelen Oxo synthesis. However, the alkyl or alkenyl group R² is preferably derived from lauryl alcohol, myristyl alcohol, cetyl alcohol, palmoleyl alcohol, stearyl alcohol, isostearyl alcohol or oleyl alcohol. Elaidyl alcohol, petroselinyl alcohol, arachidyl alcohol, gadoleyl alcohol, behenyl alcohol, erucyl alcohol and their industrial mixtures must also be named.

The cleaners according to the invention can comprise alkyl polyglycosides in proportions of 0.1 to 6% by weight, based on the total composition.

The compositions according to the invention can also comprise nitrogen-containing surfactants as other nonionic surfactants, such as fatty acid polyhydroxides, such as glucamides, and ethoxylates of alkylamines, vicinal diols and/or carboxylic acid amides having alkyl groups with 10 to 22 C atoms, preferably 12 to 18 C atoms. As a rule the degree of ethoxylation for these compounds is between 1 and 20, preferably between 3 and 10. Ethanolamide derivatives of alkanoic acids with 8 to 22 C atoms, preferably 12 to 16 C atoms, are preferred. The especially suitable compounds include lauric acid, myristic acid and palmitic acid monoethanolamides.

The amphoteric surfactants (zwitterionic surfactants) that can be used according to the invention include, among others, betaines, amino oxides, alkylamidoalkylamines, alkyl-substituted amino acids and acylated amino acids. Examples of amphoteric surfactants preferred here are betaines having the formula
(R³)(R⁴)(R⁵)N⁺CH₂COO⁻,
in which R³ is an alkyl group with 8 to 25 carbon atoms, preferably 10 to 21 carbon atoms, optionally interrupted by heteroatoms or hetero groups, and R⁴ and R⁵ are identical or different alkyl groups with 1 to 3 carbon atoms, especially C₁₀-C₁₈-alkyldimethylcarboxylmethyl betaine and C₁₁-C₁₇-alkylamidopropyl-dimethylcarboxymethyl betaine.

If the composition is to comprise cationic surfactants, they are preferably quaternary ammonium compounds having the formula
(R⁶)(R⁷)(R⁸)(R⁹)N⁺X⁻,
in which R⁶ to R⁹ represent four identical or different, especially two long-chain and two short-chain alkyl groups and X— represents an anion, especially a halide ion, such as didecyl-dimethylammonium chloride, alkyl-benzyl-didecyl-ammonium chloride, and mixtures of them. However, it is preferable for the cleaning agent composition to be free of cationic surfactants.

In selection of suitable surfactants, one must make sure that they are compatible with the enzymes used. It is preferable that the enzyme activity not decrease more than 50%, particularly not more than 30%, during a typical average working time of 15 minutes. Thus, tests have shown that sodium dodecyl sulfate (SDS), which is known to be a denaturant, reduces the activity of corolase to less than 30% within 15 minutes, even at a concentration of 1%, while the activity of xylanase decreases to less than 40% with only 0.1% SDS, so that surfactant is not preferred for use in the cleaning agents according to the invention. If that surfactant, or one with similar action, must nevertheless be used, one should preferably make sure of spatial separation during storage, such as by encapsulating the enzyme or by use of a multichamber bottle so that the enzyme and surfactant come into contact with each other only immediately before or during use. Alkyl polyglycosides, on the other hand, are well suited for use in enzyme-containing agents according to this invention. For instance, the activity of corolase remains clearly over 50% even with 3% APG 600, while xylanase actually experiences an increase in activity from the use of APG 600 (more than 150% activity with 0.1% surfactant) and still has more than 80% activity with 3% APG.

The agents according to the invention can also comprise builders. Examples of builders include alkali metal gluconates, citrates, nitrilotriacetates, carbonates and bicarbonates, especially sodium gluconate, citrate and nitrilotriacetate, as well as sodium and potassium carbonates and bicarbonates, and alkali metal and alkaline earth metal hydroxides, especially sodium and potassium hydroxides, ammonia and amines, especially monoethanolamine and triethanolamine, or mixtures of them. They also include the salts of glutaric acid, succinic acid, adipic acid, tartaric acid and benzene hexacarboxylic acid, as well as phosphonates and phosphates. The agents can comprise builders in proportions of 0.1 to 5% by weight, based on the composition.

The agents according to the invention can also comprise acids and/or alkalies. Those serve, on one hand, as pH regulators; and on the other hands the acids can contribute to removal of chalk spots from the surfaces being cleaned. The acids usable according to the invention can be inorganic mineral acids, such as hydrochloric acid, and/or C₁₋₆ mono, di, tri or polycarboxylic acids or hydroxycarboxylic acids such as, for example, formic acid, acetic acid, lactic acid, citric acid, gluconic acid, glutaric acid, succinic acid, adipic acid, tartaric acid or malic acid, as well as other organic acids such as salicylic acid or amidosulfonic acid. However, it is especially preferable to use citric acid. Mixtures of several acids can also be used. The cleaning agents according to the invention can contain acids in proportions of up to 6% by weight, based on the total composition.

The optionally usable bases include alkanolamines, for example, monoethanolamine or diethanolamine, as well as ammonium hydroxide or alkali metal hydroxides, principally sodium hydroxide. The cleaning agent according to the invention can contain bases in proportions of up to 2.5% by weight, based on the total composition.

The agents according to the invention can further contain one or more thickeners for viscosity control. Suitable thickeners include natural and synthetic polymers as well as inorganic thickeners. The usable polymers include polysaccharides or heteropolysaccharides and other natural organic thickeners, including the polysaccharide gums such as gum arabic, agar, alginates, carrageens and their salts, guar, guaran, tragacanth, gellan, ramsan, dextran or xanthan and their derivatives, such as propoxylated guar, and their mixtures, and also pectins, polyoses, carob bean meal, starches, dextrins, gelatins, and casein. Organically modified natural products such as carboxymethylcellulose and carboxymethylcellulose ethers, hydroxyethyl and hydroxypropyl cellulose and the like, or cellulose acetate, as well as carob meal ether, can be used. Homopolymeric and copolymeric polycarboxylates, principally polyacrylic and polymethacrylic compounds, and vinyl polymers, polycarboxylic acids, polyethers, polyimines or even polyamides serve as completely synthetic organic thickeners. The usable inorganic thickeners include polysilicic acids, clay minerals such as montmorillonites, zeolites, and silicic acid, as well as various nanoparticulate inorganic compounds such as nanoparticulate metal oxides, hydrated metal oxides, hydroxides, carbonates and phosphates, as well as silicates with an average particle size of 1 to 200 nm, referring to the particle diameter in the longitudinal direction, i.e., in the direction of the greatest dimension of the particles. These nanoparticulate materials can optionally, in a further embodiment of the invention, be treated with one or more surface-modification agents. The surface modification is accomplished in ways known to those skilled in the art, with monobasic and multibasic C₂₋₈-carboxylic or hydroxycarboxylic acids, functional silanes of the type (OR)₄-nSiRn (R=an organic group with functional groups such as hydroxyl, carboxy, ester, amine, epoxy, etc.), quaternary ammonium compounds or amino acids, as well as other substances usable for the purpose.

The cleaning agents according to the invention can also comprise electrolyte salts along with the thickeners named previously. Those can likewise contribute to increasing the viscosity. Electrolyte salts, in the sense of the present invention, are salts that dissociate into their ionic constituents in the aqueous media according to the invention. Salts of an inorganic acid are preferred, especially alkali metal and/or alkaline earth metal salts. The inorganic acid is preferably from the group comprising the hydrogen halide acids, nitric acid and sulfuric acid. Chlorides and sulfates are especially preferred. Within the teaching according to the invention, an electrolyte salt can also be used in the form of its corresponding acid/base pair, such as hydrochloric acid and sodium hydroxide instead of sodium chloride. Organic and/or inorganic thickeners can be used in the agent according to the invention in proportions of up to 2% by weight, based on the total composition.

The agent according to the invention can also advantageously comprise one or more water-soluble organic solvents, usually in a proportion of up to 6% by weight, based on the total composition. The solvent is used, within the teaching of the invention, as needed, particularly as a hydrotrope, viscosity regulator and/or cold stabilizer. It has a solubilizing action, especially for surfactants and electrolytes, as well as for perfumes and coloring agents, thus contributing to their incorporation. It prevents the development of liquid crystal phases, and is involved in forming clear products. The viscosity of the agent according to the invention decreases as the proportion of solvent increases. However, excessive solvent can cause too severe a drop in viscosity. Finally, the cold-clouding and clearing point of the agent according to the invention decreases with increasing proportion of solvent.

Examples of suitable solvents are saturated or unsaturated, preferably saturated, branched or unbranched C₁₋₂₀ hydrocarbons, preferably C₂₋₁₅ hydrocarbons with at least one hydroxyl group and optionally one or more ether functions, C—O—C, that is, oxygen atoms interrupting the carbon chain. However, preferred solvents are the C₂₋₆ alkylene glycols and poly-C₂₋₃-alkylene glycol ethers with an average of 1 to 9 identical or different, preferably identical, alkylene glycol groups per molecule, e.g., ethylene glycol, propylene glycol, butylene glycol, diethylene glycol, dimethoxydiglycol, dipropylene glycol, propylene glycol butyl ether, propylene glycol propyl ether, dipropylene glycol monomoethy ether and PEG. They are optionally etherified at one end with a C₁₋₆ alkanol. Other preferred solvents are the C₁₋₆ alcohols, such as methanol, ethanol, n-propanol, isopropanol, t-butanol, etc. Use of ethanol and/or isopropanol is particularly preferred.

Aside from the solvents previously described, alkanolamines and alkylbenzenesulfonates with 1 to 3 carbon atoms in the alkyl group, such as xylene or cumene sulfonate, can be used as solubilizers. Other usable hydrotropes are, for example, octyl sulfate or butyl glucoside. The agents according to the invention can contain these hydrotropes in proportions of up to 4% by weight, based on the total composition.

The agents according to the invention can comprise abrasives in on embodiment. Here solid water-soluble and non-water-soluble, preferably inorganic compounds and mixtures of them can serve as abrasive components. Those include, for example, alkali carbonates, alkali bicarbonates and alkali sulfates, alkali borates, alkali phosphates, silicon dioxide, crystalline or amorphous alkali silicates and laminar silicates, finely crystalline sodium aluminum silicates and calcium carbonate. The water-soluble abrasive components have the advantage that the medium can be rinsed off with practically no residue. Aside from these inorganic substances, abrasives obtained from living nature, such as ground nut shells or woods can be used, as well as abrasion-resistant plastics such as polyethylene beads, or tiny ceramic or glass beads. The cleaning agent according to the invention can contain abrasives in proportions up to 2% by weight, based on the total composition.

Aside from the ingredients named, the means according to the invention can comprise one or more other auxiliary substances and additives such as are particularly common in cleaning agents for hard surfaces. Those include but are not limited to UV stabilizers, corrosion inhibitors, cleaning promoters, antistatic agents, preservatives (e.g., 2-bromo-2-nitropropan-1,3-diol or an isothiazolinone-bromonitropropanediol combination), perfume, colorant, opalescence agents (such as glycol distearate) and opacifier or also skin-protecting agents such as described n EP 522 506, for example. The proportion of these additives is usually not greater than 12% by weight in the cleaning agent. The lower limit for use depends on the nature of the additive. For colorants, for instance, it can be up to 0.001% by weight or less. The proportion of auxiliary materials is preferably between 0.01 and 7% by weight, especially between 0.1 and 4% by weight.

All the colorants normally used in household cleaners can be used as colorants. All the usual perfumes can also be used as fragrances. Fruity fragrances are preferred, such as citrus, as well as pine (spruce) and mint, as well as flower fragrances. Preservatives have a biocidal action, so that it is desirable to use only very low concentrations in the cleaning agents according to the invention, and preferably no preservative at all.

It is advantageous if the means does not contain any complexing agent. It is not necessary to add a bleaching agent to the cleaning agent according to the invention.

The pH of the means according to the invention is preferably between 1 and 8, especially preferably between 2 and 5 or between 5 and 8, particularly between 2.5 and 4.5 or between 5.5 and 7.5. Here, for multiphase agents, the pH of the medium is understood to be the pH of the temporary emulsion resulting from shaking. For agents sold in multichamber bottles, the pH of the agent is the pH of the solution obtained from measuring out and mixing the specified amounts of the components stored in the different chambers. It is also in each case the pH of the cleaning solution ready for use.

If the agent is liquid, it preferably has a viscosity of up to 1000 mPas. Cleaning agents in gel or paste form can, by comparison, have viscosities of up to 150,000 mPas. The viscosity measurements are done in the Brookfield LVDV II Viscosimeter at 20° C. at a rotor speed of 20 rpm (Spindle Number 31, 100% concentration).

The cleaning agent can comprise one or more propellants (INCI Propellants), usually in a proportion of 1 to 80% by weight, preferably 1.5 to 30% by weight, especially 2 to 10% by weight, especially preferably 2.5 to 8% by weight, and most preferably 3 to 6% by weight.

Propellants according to the invention are usually propellant gases, especially liquefied or compressed gases. The choice depends on the product to be sprayed and the area of use. When compressed gases that are generally insoluble in the liquid cleaning agent are used, such as nitrogen, carbon dioxide or nitrous oxide, the working pressure decreases with each valve actuation. Liquefied gases that are soluble in the cleaning agent, or are themselves acting as the solvent, used as the propellant offer the advantage of constant working pressure and more even distribution, because the propellant evaporates in the area, increasing its volume by several hundredfold.

The following, designated as propellants by INCI, are accordingly suitable propellants: butane, carbon dioxide, dimethyl carbonate, dimethyl ether, ethane, Hydrochlorofluorocarbon 22, Hydrochlorofluoroarbon 142b, Hydrofluorocarbon 152a, Hydrofluorocarbon 134a, Hydrofluorocarbon 227ea, isobutane, isopentane, nitrogen, nitrous oxide, pentane, and propane. However, it is preferable to avoid chlorofluorocarbons (fluorochloro-hydrocarbons, CFHC) to a large extent, and especially preferably, completely, because of their harmful effect on the ozone layer of the atmosphere, which protects against hard UV radiation.

Liquefied petroleum gases are preferred propellants. Liquefied petroleum gases are gases that can be converted from the gaseous to the liquid state, usually at quite low pressures and at 20° C. In particular, though, liquefied petroleum gases are understood to mean the hydrocarbons propane, propene, butane, butene, isobutane (2-methylpropane), isobutene (2-methyl-propene, isobutylene), which occur in oil refineries as byproducts of petroleum distillation and cracking, and in natural gas production in the separation of petroleum ether, and mixtures of them.

The cleaning agent especially preferably comprises, as one or more propellants, propane, butane and/or isobutane, especially propane and butane, and most preferably propane, butane and isobutane.

The named agents and products according to the invention can comprise other components known to those skilled in the art. The sterilization, disinfection, washing, dishwashing and cleaning agents can comprise, for example, one or more components selected from the group comprising surfactants, builders, acids, alkaline substances, hydrotropes, solvents, thickeners, colorants, perfumes, corrosion inhibitors and skin-protection agents. The cleaning agent is preferably an aqueous liquid agent, but it can, for example, also be a gel, a paste or a powder.

With respect to components preferably comprised in washing and cleaning agents, explicit reference is made to the disclosure of the application DE 10309803.8, the disclosure of which in this respect is included by reference in the disclosure of the present invention.

A further object of the invention is a product comprising a composition according to the invention or a cleaning agent according to the invention and a spray dispenser.

The spray dispenser is preferably a manually actuated spray dispenser, especially selected from the group comprising aerosol spray dispensers (compressed gas container, also known, among other things, as a spray can), spray dispensers that develop their own pressure, pump spray dispensers and trigger spray dispensers, especially pump spray dispensers and trigger spray dispensers with a tank of transparent polyethylene or polyethylene terephthalate. Spray dispensers are extensively described in WO 96/04940 (Procter & Gamble) and in the US patents on spray dispensers cited therein. Reference is made to all of them in this respect and their contents are herewith included in this application. Trigger spray dispensers and pump atomizers have the advantage over pressurized gas tanks that no propellant need be used.

In a further preferred embodiment, however, the means comprising the composition is not atomized as an aerosol because in that way it could happen that small amounts of the composition could get into the respiratory tract and could initiate allergic reactions there under some circumstances. With suitable attachments, nozzles, etc. on the spray dispenser (so-called “nozzle-valves”) that allow particles to pass the, peptide can be added to the medium in an immobilized form on particles, and measured out as a cleaning foam. No particles that can get into the lungs appear when this compact foam is produced, so that the risk of inhaling allergens is essentially averted.

Further objects of the present invention are means for treating filter media, construction materials, auxiliary construction materials, textiles, furs, paper, hides or leather, comprising at least one peptide having a structure or a structural pattern of β-defensin 2.

Further objects of the present invention are filter media, construction materials, auxiliary construction materials, textiles, furs paper, hides or leather that comprise at least one peptide having a structure or structural pattern of β-defensin 2 and/or are provided with the means according to the invention.

The papers, textiles, furs, hides or leather are treated in the manner known to those skilled in the art, for example, by immersing the papers or the textiles, furs, hides or leather in a suitable concentrated solution of a means according to the invention.

The filter media, construction materials or auxiliary construction materials are treated, for example, by mechanically incorporating or by applying a suitable concentrated solution of a means according to the invention into or onto the filter media, construction materials or auxiliary construction materials.

The construction materials or auxiliary construction materials treated according to the invention are selected from adhesive or sealing compositions, compositions applied by trowel or brush, plastics, paints, colors, plaster, mortar, composition flooring material, concrete, insulation materials and priming. Especially preferred construction materials or auxiliary construction materials are seam-sealing compositions (such as silicone-containing seam-sealing compositions), wallpaper paste, plaster, carpet adhesives, silicone adhesives, and tile adhesives.

Sealing compositions and especially seam-sealing compositions typically comprise organic polymers and, in many cases, mineral or organic fillers and other additives.

Suitable polymers are, for example, thermoplastic elastomers such as are described in DE-A-3602526 by the applicant, preferably polyurethanes and acrylates. suitable polymers are also named in the Laid-Open Patent Applications DE-A-3726547, DE-A-4029504, and DE-A-4009095 of the applicant and also in DE-A-19704553 and DE-A-4233077, to which reference is made to their full extent.

The sealing compositions and especially seam-sealing compositions can comprise aqueous or organic solvents. Hydrocarbons such as cyclohexane, toluene, xylene or petroleum ether can be considered as the organic solvents. Other solvents are ketones such as methyl butyl ketone or chlorinated hydrocarbons.

The sealing compositions can also comprise other rubber-like polymers. Relatively low-molecular-weight, common commercial types of polyisobutylene, polyisoprene or polybutadiene-styrene can be considered here. It is also possible to include degraded natural rubber or neoprene rubber. Types that are still flowable at room temperature, often called “liquid rubber” can also be used here.

The sealing compositions according to the invention can be used to join together or seal off quite different materials. Use is considered primarily on concrete, on glass, on plaster and/or enamel, as well as ceramic and porcelain. But it is also possible to join or seal off shaped parts or profiles of aluminum, steel, zinc or of plastics such as PVC, polyurethanes or acrylic resins. Finally the sealing off of wood or wood materials with quite varied other materials must be mentioned.

Stability of seam-sealing compositions is generally attained by adding finely divided substances, also called fillers. They can be distinguished as materials that are inherently organic or inorganic in nature. For example, chalk, coated or uncoated, and/or zeolites can be preferred as inorganic fillers. The latter can also function as drying agents. PVC powder, for instance, is considered as an organic filler.

The fillers generally make a significant contribution to the sealing composition have the required internal stability after application, preventing the sealing composition running out or bulging out of vertical seams. The additives or fillers named can be classified as pigments and thixotropic fillers, also called briefly thixotropes.

The known thixotropes such as bentonite, kaolins or even organic compounds such as hydrogenated castor oil or its derivatives with multifunctional amines, or the reaction products of stearic or ricinoleic acid with ethylenediamine are suitable thixotropic agents. It has proved particularly favorable to include silicic acid, especially pyrolytic silicic acid. Powdered polymers that are significantly swellable can also be considered as thixotropes. Examples of those include polyacrylonitrile, polyurethane, polyvinyl chloride, polyacrylic acid esters, polyvinyl alcohols, polyvinyl acetates and the corresponding copolymers. Especially good results can be obtained with finely divided polyvinyl chloride powder. Aside from the thixotropic agents, additional adhesion promoters can also be added, such as mercaptoalkylsilane. Here it has proved to be convenient to use a mono-mercaptoalkyltrialkoxysilane. Mercaptopropyltrimoethoxysilane is common in commerce, for instance.

The properties of a seam-sealing composition can be improved still more if other components are added to the plastic powder used as the thixotropic agent. These are substances that fall under the category of plasticizers used for plastics, or swelling agents and swelling aids. For example, plasticizers of the phthalic acid ester class come into consideration. Examples of usable compounds from this substance class are dioctyl phthalate, dibutyl phthalate and benzyl butyl phthalate. Other suitable substance classes are chlorinated paraffins, alkylsulfonic acid esters of phenols or cresols and fatty acid esters.

Those low-molecular-weight organic substances that are miscible with the polymer powder and the plasticizer are usable as swelling aids. Such swelling aids can be found in the in the specific plastics and polymers handbooks for those skilled in the art. Esters, ketones, aliphatic hydrocarbons, aromatic hydrocarbons, and aromatic hydrocarbons with alkyl substituents serve as preferred swelling aids for polyvinyl chloride powder.

Known substances such as titanium dioxide, iron oxide, and carbon black are used as pigments and colorants for these applications.

It is known that stabilizers such as benzoyl chloride, acetyl chloride, toluenesulfonic acid methyl ester, carbodiimides and/or polycarbodiimides are added to the sealing compositions to improve storage stability. Olefins with 8 to 20 carbon atoms have proved to be particularly good stabilizers. Along with the stabilizing action, they can also carry out the functions of plasticizers or swelling agents. Olefins with 8 to 18 carbon atoms are preferred, especially if the double bond is in the 1,2 position. The best results are attained if the molecular structures of these stabilizers are linear.

In this way, colonization of the corresponding sealing compositions by microorganisms can be prevented deliberately and for an extended period, especially in the processed state, e.g., in the kitchen and bath.

A further preferred embodiment of the present invention is wallpaper adhesives, comprising at least one peptide with a structure of structural pattern of β-defensin 2 according to SEQ ID NO: 1 and/or its derivatives. It can be preferable to use combinations of β-defensin 2, preferably human β-defensin 2, and β-defensin 3. Wallpaper pastes of aqueous solutions of hydrocolloids such as methylcellulose, methylhydroxypropyl cellulose or water soluble starch derivatives. Aqueous dispersions of film-forming high-molecular-weight compounds, such as polyvinyl acetate can also be used, especially in combinations with the previously mentioned cellulose and starch derivatives.

With the wallpaper adhesive according to the invention it is possible to prevent or reduce microbial attack on the wallpaper, thus reducing or destroying a potential source of allergenic and/or pathogenic microorganisms.

All the known types of filter media can be used, as long as they are suitable for use in water or air filter systems. In particular, filter materials of cellulose, glass fibers, PVC fibers, polyester fibers, polyamide fibers, especially Nylon fibers, nonwoven fabrics, sintered materials and membrane filters must be named.

It was found that the peptides or peptide combinations according to the invention are suitable for reducing or almost completely preventing microbial contamination of filter media.

The following examples are intended to illustrate, but not to limit the invention.

### EXAMPLE 1

### Inhibition of Skin Microorganisms by Human β-Defensin 2

Staphylococcus epidermidis and bacillus lichenformis were grown in LB medium under aerobic conditions at 37° C. Propionibacterium acnes was grown in TBST medium under anaerobic conditions at 37° C. The concentration of human β-defensin 2 was 35.7 μg/ml. Procedure: 18-hour cultures (S. epidermidis and B. lichenformis) or 5-day cultures (P. acnes) in LB or TBST liquid media; Measurement of the cultures at A620 nm; Adjustment in PBS [phosphate-buffered saline] to the proper A620 nm for 106 CFU/ml of the test strain; Microbial content checked with a dilution series in MTP brief procedure and plating; Make up stock solution of the ingredient AWK in PBS; Put 10 μl aliquots of the test strain suspension in the PCR tubes provided for AWK and control; Add to each 10 μl aliquots of AWK or PBS; Incubate aerobically at 37° C.; Determine the microbial content of the suspensions after 8 and 24 hours, with a dilution series in the MTP brief procedure and plating; Incubation of the agar plates under proper conditions for the strain; Evaluation, by visual check of the cultured agar plates, 8 or 24 hours after the start of the test; determination of the CFU and calculation of the reduction factors.

TABLE 1

Exposure Time

| | | 8 hr | 24 hr |
|---|---|---|---|
| Defensin 2 | P. acnes | 0.95856388 | 1.83250891 |
| Defensin 2 | S. epidermis | 0.19629465 | 0.0992201 |
| Defensin 2 | B. lichenformis | 0.09275405 | 0.37161072 |

The table shows the logarithmic decrease in the cell number (RF) after incubation in nutrient medium containing β-defensin for 8 or 24 hours, compared with the control (Table 1).

The results show good inhibition of the harmful organism P. acnes by human β-defensin 2, but only slight inhibition of the useful skin microorganisms S. epidermidis and B. lichenformis.

The following exemplary formulations are intended to clarify the object of the invention without limiting it. All the statements are in % by weight based on the total composition. hBD3 is understood to be human β-defensin 3 according to SEQ ID NO:4.

1. Deodorant aerosol sprays

| | 1 | 2 | 3 | 4 | 5 | 6 |
|---|---|---|---|---|---|---|
| hBD2 (Sigma) | 0.0002 | 0.0002 | 0.0002 | 0.0002 | 0.0002 | 0.0002 |
| hBD3 | — | — | — | — | — | 0.0002 |
| Triethylcitrate | 1.00 | 5.00 | 6.00 | 1.50 | 3.00 | 1.00 |
| 2-ethylhexylglycerol | 0.50 | 0.10 | 0.50 | 0.20 | 0.30 | 0.50 |
| Phenoxyethanol | 0.30 | 0.50 | 0.10 | 0.40 | 0.60 | 0.30 |
| Perfume | 0.50 | 1.50 | 1.00 | 1.00 | 1.00 | 0.50 |
| Plant extract | 0.05 | — | 0.20 | — | 0.00 | 0.05 |
| Aroma | 0.50 | 0.01 | 0.10 | 0.10 | 0.05 | 0.50 |
| Vitamin E acetate | — | — | — | 0.05 | 0.10 | — |
| Bisabolol | — | — | 0.10 | 0.10 | — | — |
| Protectate MOD 2 | 0.40 | — | 0.20 | 0.10 | 0.20 | 0.40 |
| Protectate HR | — | — | — | — | — | — |
| Arlatone dioic acid | — | 1.00 | — | — | — | — |
| Hydrocarbon propellant | 85.00 | 60.00 | 70.00 | 75.00 | 70.00 | 85.00 |
| Alcohol denaturated | to make | to make 100 | to make 100 | to make 100 | to make | to make 100 |
| | 100 | | | | 100 | |

2. Solutions for impregnating deodorant towelettes

| | 1 | 2 | 3 | 4 |
|---|---|---|---|---|
| hBD2 (Sigma) | 0.0002 | 0.0002 | 0.0002 | 0.0002 |
| Alcohol denaturated | 50.00 | 55.00 | 60.00 | 40.00 |
| Triethylcitrate | 2.50 | 3.50 | 4.00 | 3.00 |
| PEG-40 Hydrogenated castor oil | 1.00 | 0.50 | 0.50 | 2.00 |
| 2-ethylhexylglycerol | 0.10 | 0.30 | — | — |
| Tocopheryl acetate | 0.05 | 0.20 | 0.10 | — |
| Benzophenone-2 | 0.01 | 0.01 | 0.01 | 0.05 |
| Colours approved for cosmetics | 0.0001 | 0.0005 | 0.0010 | — |
| Perfume | 0.80 | 1.00 | 2.00 | 1.50 |
| Protectate MOD 2 | 0.30 | 0.50 | — | — |
| Protectate HR | — | — | 0.20 | 0.60 |
| Phenoxyethanol | — | 1.00 | — | — |
| Aqua | to make 100 | to make 100 | to make 100 | to make 100 |

3. Deodorant aerosol sprays

| | 1 | 2 | 3 | 4 |
|---|---|---|---|---|
| hBD2 (Sigma) | 0.0002 | 0.0002 | 0.0002 | 0.0002 |
| Isopropylmyristate | 1.00 | 10.00 | 5.00 | 2.00 |
| Phenoxyethanol | 0.30 | 0.50 | 0.10 | 0.20 |
| Perfume | 0.50 | 1.50 | 1.00 | 0.50 |
| Plant extract | 0.05 | — | 0.20 | 0.50 |
| 2-ethylhexylglycerol | 0.40 | 0.10 | 1.50 | 0.80 |
| Protectate MOD 2 | 0.20 | 0.30 | — | — |
| Protectate HR | — | — | 0.10 | 0.20 |
| Arlatone dioic acid | — | — | 0.50 | — |
| Hydrocarbon propellant | 75.00 | 85.00 | 78.00 | 60.00 |
| Alcohol denaturated | to make | to make | to make | to make |
| | 100 | 100 | 100 | 100 |

4. Antiperspirant aerosol sprays

| | 1 | 2 | 3 | 4 |
|---|---|---|---|---|
| hBD2 (Sigma) | 0.0002 | 0.0002 | 0.0002 | 0.0002 |
| Aluminum chlorohydrate | 4.00 | 10.00 | | |
| Aluminum chlorohydrate activated | | | 2.00 | 10.00 |
| Disteardimonium Hectorite/Propylene | 0.50 | 1.50 | 0.80 | 1.20 |
| Carbonate | | | | |
| Perfume | 0.80 | 0.50 | 1.00 | 1.50 |
| Plant extract | 0.05 | 0.50 | 0.10 | 0.20 |
| Encapsulated Perfume/Active (Fircaps*) | 1.50 | 0.10 | 1.50 | 0.10 |
| Protectate MOD 2 | 0.05 | 0.15 | | |
| Protectate HR | | | 0.20 | 0.10 |
| Zinc gluconate | | | 0.05 | |
| Hydrocarbon propellant | 85.00 | 75.00 | 80.00 | 60.00 |
| Cyclopentasiloxane/Cylclohexasiloxane | to make | to make | to make 100 | to make |
| | 100 | 100 | | 100 |

Fircaps = Perfume-menthyl acetate mixture encapsulated in cellulose derivatives or, generally, a perfume-cooling agent mixture encapsulated in cellulose derivatives, available from Firmenich.

5. Antiperspirant aerosol sprays

| | 1 | 2 | 3 | 4 |
|---|---|---|---|---|
| hBD2 (Sigma) | 0.0002 | 0.0002 | 0.0002 | 0.0002 |
| Aluminum chlorohydrate | 4.00 | 10.00 | — | — |
| Aluminum chlorohydrate activated | — | — | 2.00 | 10.00 |
| Disteardimonium Hectorite/Propylene | 0.50 | 1.50 | 0.80 | 1.20 |
| Carbonate | | | | |
| Perfume | 0.80 | 0.50 | 1.00 | 1.50 |
| Plant extract | 0.05 | 0.50 | 0.10 | 0.20 |
| Aroma | 0.50 | 0.01 | 0.05 | 0.10 |
| Di-C1₂₋₁₃ Alkyl Malate | — | 0.50 | 0.50 | 10.00 |
| Ethylhexylpalmitat | to make 100 | 5.00 | — | — |
| Protectate MOD 2 | 0.05 | 0.15 | — | — |
| Protectate HR | — | — | 0.20 | 0.10 |
| Zinc gluconate | — | — | 0.05 | — |
| Hydrocarbon propellant | 85.00 | 75.00 | 80.00 | 60.00 |
| Cyclopentasiloxane/Cylclohexasiloxane | — | to make | to make | to make |
| | | 100 | 100 | 100 |

6. Alcoholic roll-on formulations

| | 1 | 2 | 3 | 4 | 5 |
|---|---|---|---|---|---|
| hBD2 (Sigma) | 0.0002 | 0.0002 | 0.0002 | 0.0002 | 0.0002 |
| Alcohol denaturated | 35.00 | 30.00 | 28.00 | 30.00 | 40.00 |
| Aluminum Chlorhydrate 50% solution | 16.00 | 40.00 | 16.00 | 16.00 | — |
| Aluminum Zirconium Pentachlorohydrate 40% | — | — | — | — | 45.00 |
| solution | | | | | |
| Ceteareth-12 | 2.50 | 1.50 | 2.00 | 2.00 | 2.50 |
| Ceteareth-30 | 2.50 | 2.00 | 1.50 | 2.00 | 2.50 |
| Perfume | 0.70 | 1.00 | 1.50 | 1.20 | 1.20 |
| Tocopheryl acetate | 0.05 | 0.10 | | 0.25 | 0.05 |
| Hydroxyethylcellulose | 0.50 | 0.30 | 0.40 | 0.60 | 0.50 |
| Zinc gluconate | — | 0.10 | — | — | 0.10 |
| Plant extract | — | — | 0.20 | 0.50 | 0.20 |
| Colours approved for cosmetics | 0.0005 | 0.0010 | 0.0005 | 0.0100 | 0.0001 |
| 2-ethylhexylglycerol | — | — | 0.40 | — | — |
| Protectate MOD 2 | 0.30 | 0.50 | — | — | — |
| Protectate HR | — | — | 0.20 | 0.60 | — |
| Aqua | to make 100 | to make | to make | to make | to make |
| | | 100 | 100 | 100 | 100 |

7. Roll-on emulsions

| | 1 | 2 | 3 | 4 | 5 | 6 |
|---|---|---|---|---|---|---|
| hBD2 (Sigma) | 0.0002 | 0.0002 | 0.0002 | 0.0002 | 0.0002 | 0.0002 |
| Steareth-2 | 2.50 | 3.00 | 3.00 | 2.80 | 2.80 | 2.80 |
| PPG-15 Stearyl Ether | 2.00 | 3.00 | 2.00 | 2.20 | 2.00 | 2.00 |
| Steareth-21 | 1.00 | 1.00 | 3.00 | 1.00 | 1.30 | 1.30 |
| Aluminium Chlorohydrate 50% solution | 40.00 | 40.00 | 40.00 | 40.00 | 40.00 | — |
| Aluminium Zirconium Tetrachlorohydrex | — | — | — | — | — | 63.00 |
| Gly, 35% solution | | | | | | |
| Allantoin | 0.10 | — | 0.10 | — | 0.10 | — |
| Plant extract | — | 0.50 | 0.20 | — | — | 0.20 |
| Tocopheryl acetate | 0.05 | 0.05 | 0.05 | 0.25 | 0.25 | 0.25 |
| Perfume | 1.00 | 1.50 | 1.30 | 0.80 | 1.00 | 1.20 |
| 2-ethylhexylglycerol | 0.30 | — | — | — | — | — |
| Protectate MOD 2 | 0.30 | 0.50 | — | — | — | — |
| Protectate HR | — | — | 0.20 | 0.60 | — | — |
| Arlatone dioic acid | — | — | — | — | 1.00 | — |
| Aqua | to make | to make | to make | to make | to make | to make |
| | 100 | 100 | 100 | 100 | 100 | 100 |

8. Alcoholic deodorant sticks

| | 1 | 2 | 3 | 4 |
|---|---|---|---|---|
| hBD2 (Sigma) | 0.0002 | 0.0002 | 0.0002 | 0.0002 |
| Alcohol denaturated | 40.00 | 40.00 | 35.00 | 30.00 |
| Propylene Glycol 1,2 | 30.32 | 32.32 | 32.32 | 38.00 |
| Butylene Glycol 1,3 | 12.00 | 10.00 | 15.00 | 12.00 |
| Sodium Palmitate | 3.10 | 3.50 | 2.80 | 3.10 |
| Sodium Stearate | 3.10 | 3.50 | 2.80 | 3.10 |
| Glycerol 86% | 2.00 | 1.00 | 1.70 | — |
| PPG-5-Laureth-5 | 0.50 | 1.00 | 1.00 | 0.50 |
| (Sodium Hydroxide 50%) | (2) | (2) | (2) | (2) |
| Perfume | 1.00 | 0.60 | 1.30 | 1.00 |
| Octyldodecanol | 1.00 | 0.50 | 1.00 | 0.70 |
| Phenoxyethanol | 1.00 | 0.50 | 1.00 | 0.50 |
| 2-ethylhexylglycerol | 0.50 | — | 0.30 | — |
| Tocopheryl acetate | 0.05 | — | 0.10 | 0.25 |
| Plant extract | 0.20 | — | 0.20 | 0.50 |
| PEG-40 Hydrogenated castor oil | 0.02 | — | — | 0.10 |
| Protectate MOD 2 | 0.30 | 0.50 | — | — |
| Protectate HR | — | — | 0.20 | 0.60 |
| Aqua | to make 100 | to make 100 | to make 100 | to make 100 |

9. Non-alcoholic deodorant sticks

| | 1 | 2 | 3 | 4 | 5 | 6 | 7 | 8 |
|---|---|---|---|---|---|---|---|---|
| hBD2 (Sigma) | 0.0002 | 0.0002 | 0.0002 | 0.0002 | 0.0002 | 0.0002 | 0.0002 | 0.0002 |
| hBD3 | — | — | — | — | 0.0001 | 0.0002 | 0.0001 | 0.0002 |
| PEG-8 | 40.00 | 45.00 | 50.00 | 46.00 | 40.00 | 45.00 | 50.00 | 46.00 |
| Sodium Palmitate | 2.50 | 2.50 | 2.50 | 2.50 | 2.50 | 2.50 | 2.50 | 2.50 |
| Sodium Stearate | 2.50 | 2.50 | 2.50 | 2.50 | 2.50 | 2.50 | 2.50 | 2.50 |
| Butylene Glycol 1,3 | 5.00 | 2.00 | 3.00 | 4.00 | 5.00 | 2.00 | 3.00 | 4.00 |
| PEG-14 Dimethicone | 1.00 | 2.00 | 1.50 | 1.50 | 1.00 | 2.00 | 1.50 | 1.50 |
| (Sodium Hydroxide 50%) | (1.5) | (1.2) | (1.4) | (1.3) | (1.5) | (1.2) | (1.4) | (1.3) |
| Phenoxyethanol | 1.00 | 2.00 | 0.50 | 1.00 | 1.00 | 2.00 | 0.50 | 1.00 |
| Perfume | 1.00 | 1.20 | 0.80 | 1.00 | 1.00 | 1.20 | 0.80 | 1.00 |
| 2-ethylhexylglycerol | 0.30 | — | 0.30 | — | 0.30 | — | 0.30 | — |
| Steareth-10 | 0.20 | — | 0.20 | 0.20 | 0.20 | — | 0.20 | 0.20 |
| Plant extract | 0.20 | 0.50 | — | 0.30 | 0.20 | 0.50 | — | 0.30 |
| Protectate MOD 2 | 0.30 | 0.50 | — | — | 0.30 | 0.50 | — | — |
| Protectate HR | — | — | 0.20 | 0.60 | — | — | 0.20 | 0.60 |
| Aqua | to make | to make | to make | to make | to make | to make | to make | to make |
| | 100 | 100 | 100 | 100 | 100 | 100 | 100 | 100 |

10. Antiperspirant sticks

| | 1 | 2 | 3 | 4 |
|---|---|---|---|---|
| hBD2 (Sigma) | 0.0002 | 0.0002 | 0.0002 | 0.0002 |
| PPG-14 Butylether | 15.00 | 18.00 | 12.00 | 19.00 |
| Hydrogenated castor oil | 1.00 | 1.50 | 2.00 | 1.50 |
| Stearyl alcohol | 20.00 | 18.00 | 15.00 | 18.00 |
| Ceteareth-30 | 3.00 | 2.00 | 4.00 | — |
| Isoceteth-20 | — | — | — | 2.50 |
| Perfume | 1.00 | 1.20 | 0.80 | 1.50 |
| Aluminumchlorohydrate | 20.00 | 22.00 | 18.00 | — |
| Aluminium Zirconium tetrachlorohydrex Gly | — | — | — | 22.00 |
| Allantoin | 0.10 | — | — | 0.10 |
| Cocoglycerides | 4.00 | 6.00 | 3.00 | 5.00 |
| Talc | 3.00 | 2.00 | 5.00 | 3.00 |
| Plant extract | 0.20 | 0.50 | — | — |
| Tocopheryl acetate | 0.20 | — | 0.50 | 0.10 |
| 2-ethylhexylglycerol | 0.30 | — | — | — |
| Protectate MOD 2 | 0.30 | 0.50 | — | — |
| Protectate HR | — | — | 0.20 | — |
| Cyclopentasiloxane | to make 100 | to make 100 | to make 100 | to make 100 |
| | 5 | 6 | 7 | 8 |
| hBD2 (Sigma) | 0.0002 | 0.0002 | 0.0002 | 0.0002 |
| Hexyldecanol | 10.00 | 12.00 | 10.00 | 8.00 |
| PPG-14 Butylether | 6.00 | 5.00 | 6.00 | 8.00 |
| Hydrogenated castor oil | 4.00 | 5.00 | 6.00 | 5.00 |
| Stearyl alcohol | 12.00 | 14.00 | 11.00 | 16.00 |
| Cetyl alcohol | 6.00 | 5.00 | 6.00 | 3.00 |
| PEG-20 Glycerol stearate | 5.00 | 4.00 | 6.00 | 4.00 |
| Ceteareth-30 | 3.00 | 1.00 | 3.00 | — |
| Perfume | 1.00 | 1.20 | 0.80 | 1.00 |
| Aluminumchlorohydrate | 20.00 | 20.00 | 18.00 | — |
| Aluminium Zirconium tetrachlorohydrex Gly | — | — | — | 23.00 |
| Talc | 8.00 | 5.00 | 8.00 | 7.00 |
| Plant extract | 0.20 | 0.50 | — | — |
| Tocopheryl acetate | 0.25 | — | 0.50 | — |
| 2-Ethylhexylglycerol | 0.30 | — | — | — |
| Protectate MOD 2 | 0.30 | 0.50 | — | — |
| Protectate HR | — | — | 0.20 | — |
| Cyclopentasiloxane | to make | to make | to make | to make |
| | 100 | 100 | 100 | 100 |

11. Deodorant sprays in pump dispenser (no propellant gas)

| | 1 | 2 | 3 | 4 |
|---|---|---|---|---|
| hBD2 (Sigma) | 0.0002 | 0.0002 | 0.0002 | 0.0002 |
| Alcohol denaturated | 50.00 | 55.00 | 60.00 | 40.00 |
| Triethylcitrate | 2.50 | 3.50 | 4.00 | 3.00 |
| PEG-40 Hydrogenated castor oil | 1.00 | 0.50 | 0.50 | 2.00 |
| 2-Ethylhexylglycerol | 0.10 | 0.50 | — | — |
| Tocopheryl Acetate | 0.05 | 0.20 | 0.10 | — |
| Benzophenone-2 | 0.01 | 0.01 | 0.01 | 0.05 |
| Colours approved for cosmetics | 0.0001 | 0.0005 | 0.0010 | — |
| Perfume | 0.80 | 1.00 | 2.00 | 1.50 |
| Protectate MOD 2 | 0.30 | 0.50 | — | — |
| Protectate HR | — | — | 0.20 | 0.60 |
| Phenoxyethanol | — | 1.00 | — | — |
| Aqua | to make 100 | to make 100 | to make 100 | to make 100 |

12. Antiperspirant-PIT emulsions in pump dispenser (no propellant gas)

| | 1 | 2 | 3 | 4 |
|---|---|---|---|---|
| hBD2 (Sigma) | 0.0002 | 0.0002 | 0.0002 | 0.0002 |
| Aluminum chlorohydrate 50% solution | 30.00 | 40.00 | 35.00 | 40.00 |
| Dicaprylyl ether | 10.00 | 10.00 | 8.00 | 9.00 |
| Glycerol 86% | 5.00 | 3.00 | 5.00 | 3.00 |
| Beheneth-10 | 3.30 | 4.00 | 3.50 | 4.00 |
| Cetearyl isononanoate | — | — | 4.00 | 5.00 |
| Hexyldecanol/Hexyldecyl Laurate | 3.00 | 5.00 | — | — |
| Perfume | 1.00 | 0.80 | 1.20 | 1.00 |
| Plant extract | 0.20 | — | 0.50 | — |
| Polysorbate 20/Linoleic Acid | 0.20 | 0.20 | 0.50 | — |
| Allantoin | 0.10 | — | — | 0.20 |
| Protectate MOD 2 | 0.30 | 0.50 | — | — |
| Protectate HR | — | — | 0.20 | — |
| Aqua | to make 100 | to make 100 | to make 100 | to make 100 |

13. Clear antiperspirant gels

| | 1 | 2 | 3 | 4 | 5 |
|---|---|---|---|---|---|
| hBD2 (Sigma) | 0.0002 | 0.0002 | 0.0002 | 0.0002 | 0.0002 |
| hBD3 | — | — | — | — | 0.0002 |
| Propylene Glycol 1,2 | 18.00 | 23.00 | 18.00 | 20.00 | 23.00 |
| Aluminum Chlorohydrate 50% solution | 40.00 | 40.00 | 40.00 | 40.00 | 40.00 |
| Cyclopentasiloxane | 14.20 | 14.20 | 14.20 | 14.20 | 14.20 |
| Alcohol denaturated | 5.00 | 10.00 | 8.00 | 10.00 | 10.00 |
| BIS-PEG/PPG-14/14 Dimethicone | 3.50 | 2.50 | 3.20 | 3.00 | 2.50 |
| Perfume | 0.60 | 0.60 | 1.00 | 1.30 | 0.60 |
| Plant extract | 0.50 | — | — | — | — |
| Allantoin | — | 0.10 | — | — | 0.10 |
| 2-Ethylhexylglycerol | 0.30 | — | — | — | — |
| Protectate MOD 2 | 0.30 | 0.50 | — | — | 0.50 |
| Protectate HR | — | — | 0.20 | — | — |
| Aqua | to make | to make | to make | to make | to make |
| | 100 | 100 | 100 | 100 | 100 |

The refractive index of the water phase must be matched to the refractive index of the oil phase to get clear gels. Water or propylene glycol act as the variables.

14. Clear deodorant gels

| | 1 | 2 | 3 | 4 | 5 | 6 | 7 | 8 |
|---|---|---|---|---|---|---|---|---|
| hBD2 (Sigma) | 0.0002 | 0.0002 | 0.0002 | 0.0002 | 0.0002 | 0.0002 | 0.0002 | 0.0002 |
| hBD3 | — | — | — | — | 0.0002 | 0.0002 | 0.0002 | 0.0002 |
| Alcohol denaturated | 30.00 | 40.00 | 50.00 | 60.00 | 30.00 | 40.00 | 50.00 | 60.00 |
| Ceteareth-12 | 1.50 | — | 2.00 | — | 1.50 | — | 2.00 | — |
| Ceteareth-30 | 2.50 | — | 2.00 | — | 2.50 | — | 2.00 | — |
| PEG-40 Hydrogenated castor | — | 3.00 | — | 2.00 | — | 3.00 | — | 2.00 |
| oil | | | | | | | | |
| Carbomer | 0.30 | 0.50 | 0.80 | 1.00 | 0.30 | 0.50 | 0.80 | 1.00 |
| Neutralisation Agent | q.s.* | q.s.* | q.s.* | q.s.* | q.s.* | q.s.* | q.s.* | q.s.* |
| Perfume | 0.60 | 0.60 | 1.00 | 1.30 | 0.60 | 0.60 | 1.00 | 1.30 |
| Plant extract | 0.50 | — | 0.20 | — | 0.50 | — | 0.20 | — |
| Ethylhexylglycerol | — | 0.30 | — | — | — | 0.30 | — | — |
| Protectate MOD 2 | 0.30 | 0.50 | — | — | 0.30 | 0.50 | — | — |
| Protectate HR | — | — | 0.40 | — | — | — | 0.40 | — |
| Phenoxyethanol | — | 1.00 | — | — | — | 1.00 | — | — |
| Aqua | to make 100 | to make 100 | to make | to make | to make | to make | to make | to make |
| | | | 100 | 100 | 100 | 100 | 100 | 100 |

The thickener (Carbomer) must be adjusted to the desired pH with a suitable neutralizing agent (triethanolamine, 2-amino-2-methylpropanol-1 (AMP), sodium hydroxide, lithium hydroxide.

15. Antiperspirant cream

| | 1 | 2 | 3 | 4 |
|---|---|---|---|---|
| hBD2 (Sigma) | 0.0002 | 0.0002 | 0.0002 | 0.0002 |
| Aluminum Chlorohydrate 50% solution | 40.00 | 40.00 | 35.00 | 45.00 |
| Glyceryl Stearate | 5.00 | 4.50 | 5.50 | 6.00 |
| Cetyl Alcohol | 2.00 | — | 3.00 | 1.50 |
| Behenyl Alcohol | 1.50 | 4.00 | 3.50 | 5.00 |
| Dimethicone | 2.00 | 1.50 | 2.50 | 3.00 |
| Ceteareth-12 | 1.50 | 2.00 | 2.50 | 1.30 |
| Ceteareth-20 | 1.50 | 2.00 | 2.50 | 1.30 |
| Hexyldecanol/Hexyldecyl Laurate | 3.00 | 4.00 | 2.50 | 2.40 |
| Cyclopentasiloxane | 1.50 | 3.00 | 2.00 | 1.00 |
| Plant extract | 0.20 | 0.50 | — | — |
| Tocopheryl Acetate | 0.05 | 0.25 | — | — |
| Perfume | 0.80 | 1.00 | 1.50 | 2.00 |
| Allantoin | 0.10 | 0.10 | — | — |
| Preservative system | 0.05 | 0.50 | 0.50 | 0.50 |
| 2-ethylhexylglycerol | 0.30 | — | — | — |
| Protectate MOD 2 | 0.30 | 0.50 | — | — |
| Protectate HR | — | — | 0.20 | — |
| Aqua | to make 100 | to make 100 | to make 100 | to make 100 |

17. Impregnating solutions (PIT solutions) for antiperspirant towelettes

| | 1 | 2 | 3 | 4 |
|---|---|---|---|---|
| hBD2 (Sigma) | 0.001 | 0.001 | 0.001 | 0.001 |
| Aluminum | 30.00 | 40.00 | 35.00 | 40.00 |
| chlorohydrate 50% | | | | |
| solution | | | | |
| Dicaprylyl Ether | 10.00 | 10.00 | 8.00 | 9.00 |
| Glycerol 86% | 5.00 | 3.00 | 5.00 | 3.00 |
| Beheneth-10 | 3.30 | 4.00 | 3.50 | 4.00 |
| Cetearyl isononanoate | — | — | 4.00 | 5.00 |
| Hexyldecanol/ | 3.00 | 5.00 | — | — |
| Hexyldecyl Laurate | | | | |
| Perfume | 1.00 | 0.80 | 1.20 | 1.00 |
| Plant extract | 0.20 | — | 0.50 | — |
| Polysorbate 20/ | 0.20 | 0.20 | 0.50 | — |
| Linoleic Acid | | | | |
| Allantoin | 0.10 | — | — | 0.20 |
| 2-ethylhexylglycerol | 0.30 | — | — | — |
| Protectate MOD 2 | 0.30 | 0.50 | — | — |
| Protectate HR | — | — | 0.20 | — |
| Preservative System | 0.50 | 0.20 | 1.00 | 0.50 |
| Aqua | to make | to make | to make | to make |
| | 100 | 100 | 100 | 100 |

1. Oil in Water Emulsions

1. Series of Examples

| | 1 | 2 | 3 |
|---|---|---|---|
| Thistle oil | 3.00 | 3.00 | 3.00 |
| Myritol 318 | 5.00 | 5.00 | 5.00 |
| Novata AB | 2.00 | 2.00 | 2.00 |
| Lanette 22 | 1.00 | 1.00 | 1.00 |
| Cutina MD | 2.00 | 2.00 | 2.00 |
| Stenol 1618 | 1.00 | 1.00 | 1.00 |
| Isopropylstearate | 4.00 | 4.00 | 4.00 |
| Cetiol SB 45 | 2.00 | 2.00 | 2.00 |
| Baysilon M350 | 1.00 | 1.00 | 1.00 |
| Controx KS | 0.05 | 0.05 | 0.05 |
| Propylparaben | 0.20 | 0.20 | 0.20 |
| Dow Corning Fluid 1501 | 1.00 | 1.00 | 1.00 |
| Dry Flo Plus | 1.00 | 1.00 | 1.00 |
| TiO2 | 0.50 | 0.50 | 0.50 |
| Hexandiol | 6.00 | 3.00 | |
| Propylenglycol | 5.00 | 5.00 | 5.00 |
| Glycerol | 5.00 | 3.00 | 3.00 |
| Methylparaben | 0.20 | 0.20 | 0.20 |
| Tego Carbomer | 0.40 | 0.40 | 0.40 |
| DS-HCN | 5.00 | 5.00 | 5.00 |
| Spirulina extract 3002 | 1.00 | | |
| hBD2 (Sigma) | 0.0002 | 0.0003 | 0.0002 |
| Perfume | 0.10 | 0.10 | 0.10 |
| Aqua | to make 100 | to make 100 | to make 100 |

2. Series of Examples

| | 1 | 2 | 3 |
|---|---|---|---|
| Cetiol SN | 4.00 | 4.00 | 4.00 |
| Mineral oil | 6.00 | 6.00 | 6.00 |
| Cutina CBS | 2.00 | 2.00 | 2.00 |
| Edenor L2 SM | 1.50 | 1.50 | 1.50 |
| Emulgin B3 | 1.00 | 1.00 | 1.00 |
| Baysilon M 350 | 1.00 | 1.00 | 1.00 |
| Tocoperylacetat | 0.50 | 0.50 | 0.50 |
| Propylparaben | 0.30 | 0.30 | 0.30 |
| Almond oil | 2.00 | 2.00 | 2.00 |
| Permulen Tr-1 | 0.27 | 0.27 | 0.27 |
| Glycerol | 5.00 | 3.00 | 3.00 |
| Lactic acid 80% | 0.26 | 0.26 | 0.26 |
| Propylenglycol | 5.00 | 5.00 | 5.00 |
| Methylparaben | 0.30 | 0.30 | 0.30 |
| Phenoxyethanol | 0.90 | 0.90 | 0.90 |
| Panthenol | 0.50 | — | — |
| Laminaria Digitata Extract | 1.00 | — | — |
| Sodium chloride | 0.05 | 0.05 | 0.05 |
| Seppigel 305 | 0.50 | 0.50 | 0.50 |
| Silk Protein | 0.25 | 0.25 | 0.25 |
| Keltrol SF | 0.20 | 0.20 | 0.20 |
| hBD2 (Sigma) | 0.0002 | 0.0002 | 0.0002 |
| Perfume | 0.30 | 0.30 | 0.30 |
| Aqua | to make 100 | to make 100 | to make 100 |

3. Series of Examples

| | 1 | 2 | 3 |
|---|---|---|---|
| Lipoid S75-3 | 0.50 | 0.50 | 0.50 |
| Isopropylstearate | 4.00 | 4.00 | 4.00 |
| Cetiol B | 2.00 | 2.00 | 2.00 |
| Tocopherylacetat | 0.50 | 0.50 | 0.50 |
| Cutina MD | 1.00 | 1.00 | 1.00 |
| Lanette 22 | 2.00 | 2.00 | 2.00 |
| Baysilone M 350 | 0.50 | 0.50 | 0.50 |
| Propylparaben | 0.20 | 0.20 | 0.20 |
| Dow Corning 9040 | 1.00 | 1.00 | 1.00 |
| Glycerol | 4.50 | 3.00 | 3.00 |
| Hexandiol | 6.00 | 3.00 | — |
| Methylparaben | 0.20 | 0.20 | 0.20 |
| Tego Carbomer 140 | 0.30 | 0.30 | 0.30 |
| DS-HCN | 5.00 | 5.00 | 5.00 |
| Algal extract | 1.00 | — | — |
| Phytosomes | 0.20 | 0.20 | 0.20 |
| Lipochroman-6 | 0.01 | 0.01 | 0.01 |
| Propylenglycol | 5.00 | 5.00 | 5.00 |
| Matrixyl | 3.00 | — | — |
| Matrixyl 3000 | — | 3.00 | — |
| Simugel NS | 1.50 | 1.50 | 1.50 |
| TiO₂ | 0.50 | 0.50 | |
| hBD2 (Sigma) | 0.0002 | 0.0002 | 0.0002 |
| Perfume | 0.35 | 0.35 | 0.35 |
| Aqua | to make 100 | to make 100 | to make 100 |

4. Series of Examples

| | 1 | 2 | 3 | 4 | 5 |
|---|---|---|---|---|---|
| Montanov 68 | 5.00 | 5.00 | 5.00 | 5.00 | 5.00 |
| Myritol 316 | 5.00 | 5.00 | 5.00 | 5.00 | 5.00 |
| Cetiol SB 45 | 0.50 | 0.50 | 0.50 | 0.50 | 0.50 |
| Novata AB | 2.00 | 2.00 | 2.00 | 2.00 | 2.00 |
| Stenol 1618 | 1.00 | 1.00 | 1.00 | 1.00 | 1.00 |
| Baysilon M 350 | 0.50 | 0.50 | 0.50 | 0.50 | 0.50 |
| Tocoperylacetat | 0.50 | 0.50 | 0.50 | 0.50 | 0.50 |
| Controx KS | 0.25 | 0.25 | 0.25 | 0.25 | 0.25 |
| Parsol SLX | — | 4.00 | 4.00 | — | — |
| Parsol HS | — | 2.00 | 2.00 | 2.00 | — |
| Neo Heliopan AP | — | — | — | 1.00 | — |
| Parsol 340 | — | — | — | 5.00 | — |
| Uvinul MBC 95 | 2.00 | — | — | — | — |
| Parsol 1789 | 1.00 | 1.80 | 1.80 | — | — |
| Propylparaben | 0.20 | 0.20 | 0.20 | 0.20 | 0.20 |
| Tego Carbomer 140 | 0.50 | 0.50 | 0.50 | 0.50 | 0.50 |
| Hexandiol | 6.00 | 6.00 | 3.00 | 6.00 | — |
| Talkum Pharam G | 0.50 | 0.50 | 0.50 | 0.50 | 0.50 |
| Methylparaben | 0.20 | 0.20 | 0.20 | 0.20 | 0.20 |
| Glycerol | 4.50 | 4.50 | 3.00 | 4.50 | 3.00 |
| Dry Flo Plus | 1.00 | 1.00 | 1.00 | 1.00 | 1.00 |
| Natipide 2 PG | 1.00 | 1.00 | 1.00 | 1.00 | 1.00 |
| Gatuline R/C | 2.00 | 2.00 | 2.00 | 2.00 | 2.00 |
| Hydrolyzed protein | 5.00 | 5.00 | 5.00 | 5.00 | 5.00 |
| DS-HCN | 2.00 | 2.00 | 2.00 | 2.00 | 2.00 |
| Trilon A | 0.10 | 0.10 | 0.10 | 0.10 | 0.10 |
| Phenoxyethanol | 0.40 | 0.40 | 0.40 | 0.40 | 0.40 |
| hBD2 (Sigma) | 0.0001 | 0.0002 | 0.0003 | 0.0002 | 0.0002 |
| Perfume | 0.40 | 0.40 | 0.40 | 0.40 | 0.40 |
| Aqua | to make 100 | to make | to make 100 | to make | to make 100 |
| | | 100 | | 100 | |

5. Series of Examples

| | 1 | 2 | 3 |
|---|---|---|---|
| Montanov L | 3.00 | 3.00 | 3.00 |
| Cetiol B | 6.00 | 6.00 | 6.00 |
| Myritol 318 | 3.00 | 3.00 | 3.00 |
| Stenol 1618 | 1.00 | 1.00 | 1.00 |
| Cutina MD | 0.50 | 0.50 | 0.50 |
| Baysilon M 350 | 0.50 | 0.50 | 0.50 |
| Propylparaben | 0.20 | 0.20 | 0.20 |
| Dow Corning 245e | 1.50 | 1.50 | 1.50 |
| Glycerol | 5.00 | 5.00 | 5.00 |
| Karion F | 3.00 | 3.00 | 3.00 |
| Methylparaben | 0.20 | 0.20 | 0.20 |
| Dry Flo Plus | 0.50 | 0.50 | 0.50 |
| Tego Carbomer 140 | 0.10 | 0.10 | 0.10 |
| DS-HCN | 2.00 | 2.00 | 2.00 |
| Laminaria Digitata Extract | 0.50 | 0.50 | 0.50 |
| Simugel NS | 1.00 | 1.00 | 1.00 |
| hBD2 (Sigma) | 0.0002 | 0.0002 | 0.0002 |
| Perfume | 0.20 | 0.20 | 0.20 |
| Aqua | to make 100 | to make 100 | to make 100 |

2. Water-in-Oil Emulsion

| | 1 | 2 | 3 |
|---|---|---|---|
| Lameform TGI | 3.0 | 3.0 | 3.0 |
| PEG-45/Dodecyl Glycol Copolymer | 0.5 | 0.5 | 0.5 |
| Microcrystalline Wax | 3.0 | 3.0 | 3.0 |
| Bis-Diglyceryl Polyacyladipate-2 | 1.0 | 1.0 | 1.0 |
| Paraffin oil | 8.0 | 8.0 | 8.0 |
| Vaseline | 2.0 | 2.0 | 2.0 |
| Vitamin E acetat | 2.0 | 2.0 | 2.0 |
| Methylparaben | 0.3 | 0.3 | 0.3 |
| Propylparaben | 0.3 | 0.3 | 0.3 |
| Isopropylisostearate | 8.0 | 8.0 | 8.0 |
| Glycerol | 5.0 | 3.0 | 3.0 |
| Magnesium sulfate | 0.5 | 0.5 | 0.5 |
| hBD2 (Sigma) | 0.0003 | 0.0002 | 0.0002 |
| Lactic acid 80% ig | 0.560 | 0.560 | 0.560 |
| Algal extract | 1.0 | — | — |
| Panthenol | 0.5 | 1.0 | 0.5 |
| Calendula Officinalis Flower Extract | 0.3 | — | — |
| Propyleglycol | 0.5 | — | — |
| Perfume | 0.2 | 0.2 | 0.2 |
| Aqua | to make | to make | to make |
| | 100 | 100 | 100 |

3. Cleaning Preparations

1. Series of Examples

| | 1 | 2 | 3 |
|---|---|---|---|
| Dipropylenglycol | 10.00 | 10.00 | 10.00 |
| Chlorhexidindigluconat | 1.00 | 1.00 | 1.00 |
| Synperonic PE7L 64 | 3.00 | 3.00 | 3.00 |
| D-Panthenol | 0.50 | 0.50 | 0.50 |
| Hydagen CMF | 3.00 | 3.00 | 3.00 |
| PEG-40 Hydrogenated castor | 0.50 | 0.50 | 0.50 |
| oil/Trideceth 9/Propylen | | | |
| Glycol | | | |
| Chlorella Vulgaris extract | 0.50 | 0.50 | 0.50 |
| hBD2 (Sigma) | 0.0004 | 0.0003 | 0.0002 |
| Perfume | 0.20 | 0.20 | 0.20 |
| Aqua | to make 100 | to make 100 | to make 100 |

2. Series of Examples

| | 1 | 2 | 3 |
|---|---|---|---|
| Carbopol ETD 2001 | 1.40 | 1.40 | 1.40 |
| Sorbitol | 2.10 | 2.10 | 2.10 |
| Sodium benzoate | 0.40 | 0.40 | 0.40 |
| Plantacare 2000UP | 7.50 | 7.50 | 7.50 |
| Cocoamidopropyl beatine | 3.40 | 3.40 | 3.40 |
| Texapon SB 3 | 5.00 | 5.00 | 5.00 |
| Cetiol HE | 0.50 | 0.50 | 0.50 |
| Lamesoft PO 65 | 5.00 | 5.00 | 5.00 |
| Controx KS | 0.05 | 0.05 | 0.05 |
| Ajidew NL 50 | 1.60 | 1.60 | — |
| Pantolacton | 1.00 | 1.00 | — |
| Trilon B | 0.25 | 0.25 | 0.25 |
| Sodium lactate | 1.80 | — | — |
| D-Panthenol | 0.50 | 0.50 | 0.50 |
| hBD2 (Sigma) | 0.0003 | 0.0002 | 0.0004 |
| Perfume | 0.40 | 0.40 | 0.40 |
| Aqua | to make 100 | to make 100 | to make 100 |

4. Water in Silicone Emulsion

| | 1 | 2 | 3 |
|---|---|---|---|
| Belsil DM 100 | 2.50 | 2.50 | 2.50 |
| Dow Corning 245 Fluid | 25.00 | 25.00 | 25.00 |
| Abil EM 90 | 2.00 | 2.00 | 2.00 |
| Sodium chloride | 2.00 | 2.00 | 2.00 |
| Perfume | 0.30 | 0.30 | 0.30 |
| Symdiol 68 | 0.30 | 0.30 | 0.30 |
| Phenoxyethanol | 0.40 | 0.40 | 0.40 |
| hBD2 (Sigma) | 0.0002 | 0.0002 | 0.0002 |
| Aqua | to make 100 | to make 100 | to make 100 |

| Trade name | INCI name | Supplier/Manufacturer |
|---|---|---|
| Abil EM 90 | Cetyl PEG/PPG-10/1 Dimethicone | Degussa |
| Acnacidol PG | Sebacic acid, 10-hydroxydecanoic | Vincience |
| | acid, 1,10-decanediol | |
| AJIDEW ® NL 50 | Sodium PCA | AJINOMOTO |
| Belsil DM 100 | Polydimethylsiloxan | Wacker |
| Carbopol ETD 2020 | Acrylates/C₁₀₋₃₀ Alkyl Acrylate Cross- | Noveon |
| | polymer | |
| Carbopol ETD 2001 | Carbomer | Noveon |
| CETIOL ® B | Dibutyladipate | Cognis |
| CETIOL ® SN | Cetearyl isononanoate | Cognis |
| CETIOL ® SB 45 | Shea butter, Butyrospermium Parkii | Cognis |
| Controx KS | Tocopherol, Hydrogenated Palm | Cognis |
| | Glycerides Citrate | |
| Cutina CBS | Glyceryl Stearate/Cetearyl Alcohol/ | Cognis |
| | Cetyl Palmitate/Cocoglycerides | |
| Cutina MD | Glyceryl Stearate | Cognis |
| Dow Corning 1501 | Cyclomethicone, Dimethiconol | Dow Corning |
| Fluid | | |
| Dow Corning 245 | Cyclomethicone, | Dow Corning |
| Fluid | | |
| Dry Flo Plus | Aluminium Starch Octenylsuccinate | National Starch |
| DSH CN | Water, Dimethylsilanol Hyaluronate | Exsymol |
| Edenor L2 SM | Palmitic acid, Stearic acid | Cognis |
| Eumulgin B3 | C₁₆₋₁₈ ethoxylated fatty alcohol (30 | Cognis |
| | EO) | |
| Gatuline R/C | Fagus Silvatica Extract in water | Gattefosse |
| Hydagen CMF | Chitosan Glycolate | Cognis |
| Karion F | Sorbitol | Merck |
| Keltrol SF | Xanthan Gum | CP Kelco |
| Lameform TGI | Polyglyceryl-3 Diisostearate | Cognis |
| LAMESOFT ® PO 65 | Coco-Glucoside, Glyceryl Oleate, | Cognis |
| | Water | |
| LANETTE ® 22 | Behenyl Alcohol | Cognis |
| Lipoid S75-3 | Hydrogenated Lecithin | Lipoid GmbH |
| Lipochroman-6 | DIMETHYLMETHOXY | Lipotec |
| | CHROMANOL | |
| Madecassoside | Centella Asiatica Extract | DSM |
| Matrixyl | Aqua, Palmitoyl Pentapeptide-3 | Sederma |
| Matrixyl 3000 | Glycerol, Aqua, Butylene Glycol, | Sederma |
| | Carbomer, Polysorbate 20, Palmitoyl | |
| | Oligopeptide, Palmitoyl Tetrapeptide-1 | |
| Montanov 68 | Cetearyl Alkohol, Cetearyl Glucoside | Seppic |
| Montanov L | C14-22 Alcohols/C₁₂₋₂₀ Alkyl | Seppic |
| | Glucoside | |
| Myritol 316 | Caprylic/Capric Triglyceride | Cognis |
| Natipide 2 PG | Propylene Glycol, Lecithin 1% | Rhone Poulenc |
| Novata AB | Cocoglycerides | Cognis |
| Neo Heliopan AP | Disodium Phenyl Dibenzimidazole | Symrise |
| | Tetrasulfonate | |
| Parsol 340 | Octocrylene | L.C. United |
| Parsol 1789 | Butylmethoxydibenzoylmethane | L.C. United |
| Parsol HS | Phenylbenzimidazole-sufonic acid | L.C. United |
| Parsol SLX | Polysilicone-15 | L.C. United |
| Pemulen TR 1 | Acrylates/C₁₀₋₃₀ Alkyl Acrylate Cross- | |
| | polymer | |
| PHENONIP ® | Phenoxyethanol, Methylparaben, | NIPA |
| | Ethylparaben, Propylparaben, | |
| | Butylparaben, ca. 28% Active | |
| | substance | |
| Photosomes | Plancton Extract | AGI Dermatics |
| Phycosaccharide AI | Water (and) Hydrolyzed Algin | Codif |
| PLANTACARE ® 818 | Coco Glucoside, ca. 50% Active | Cognis |
| UP | substance | |
| PLANTAREN ® 1200 | Lauryl Glucoside, ca. 50% Active | Cognis |
| | substance | |
| PLANTAREN ® 2000 | Decyl Glucoside, ca. 50% Active | Cognis |
| UP | substance | |
| SEPIGEL ® 305¹⁵ | Polyacrylamide, C₁₃-C₁₄ Isoparaffin, | SEPPIC |
| | Laureth-7 | |
| Silymarin Phytosome | Silybum Marianum Extract and | Indena SpA |
| | Phospholipids | |
| Simulgel NS | Hydroxyethyl Acrylate/Sodium | Seppic |
| | Acryloyldimethyl Taurate Copolymer/ | |
| | Squalane/Polysorbate 60 | |
| Stenol 1618 | Cetearyl alcohol | Cognis |
| Symdiol 68 | 1,2-Hexanediol, Caprylyl Glycol | Symrise |
| Synperonic PE7L 64 | Poloxamer-184 | BASF |
| Tego Carbomer 140 | Carbomer | Degussa |
| TEXAPON ® SB3 | Sodium Laureth Sulfosuccinate, Citric | Cognis |
| | Acid, Aqua (ca. 40% Active | |
| | substance) | |
| Trilon A | Nitrilotriacetic acid, trisodium salt | BASF |
| Trilon B | Ethylendiamine tetra acetic acid | BASF |
| | tetrasodium salt | |
| Uvinul MBC 95 | 4-Methylbenzylidene Camphor | BASF |

Various modifications of the invention, in addition to those described herein, will be apparent to those skilled in the art from the foregoing description. Such modifications are also intended to fall within the scope of the appended claims.

What is claimed:

1. A composition comprising at least one β-defensin 2 polypeptide having SEQ ID NO:1, or a derivative thereof, wherein X is selected independently from the group of the essential and non-essential amino acids.

2. The composition of claim 1, wherein X is Cys, Gly, Thr, or Lys.

3. The composition of claim 1, wherein X is independently Thr or Gly at position 29, Cys at position 8, 15, 20, 30, 37, or 38, or Lys at position 39 or 40.

4. The composition of claim 1, comprising at least one β-defensin 2 polypeptide having SEQ ID NO:2, or a derivative thereof.

5. The composition of claim 1, comprising at least one β-defensin 2 polypeptide having SEQ ID NO:3, or a derivative thereof.

6. The composition of claim 1, 4, or 5, wherein the β-defensin 2 polypeptide or derivative thereof is present at a concentration of 10 ng/ml to 100 μg/ml.

7. The composition of claim 1, wherein the β-defensin 2 polypeptide or derivative thereof is present at a concentration of 0.00001 to 50% by weight.

8. The composition of claim 1, further comprising at least one β-defensin 3 polypeptide having SEQ ID NO:4 or 5, or a derivative thereof.

9. The composition of claim 8, wherein the β-defensin 3 polypeptide or derivative thereof is present at a concentration of 0.00001 to 50% by weight.

10. The composition of claim 1, further comprising at least one deodorizing agent or antiperspirant.

11. The composition of claim 1, further comprising at least one sebum regulator.

12. The composition of claim 1, further comprising at least one anti-inflammatory agent.

13. The composition of claim 1, further comprising at least one prebiotic agent.

14. A method for reducing or preventing microbial contamination of washing agents, cleaning agents, cosmetics, pharmaceuticals, filter media, construction materials, or water treatment agents, comprising admixing an effective amount of the composition of claim 1 with the washing agents, cleaning agents, cosmetics, pharmaceuticals, filter media, construction materials, or water treatment agents.

15. The method of claim 14, wherein the cosmetics are deodorants, dental care agents, or oral care agents.

16. The method of claim 14, wherein the water treatment agents are used to treat water in closed-loop circulation.

17. The method of claim 14, wherein the water treatment agents are used to treat water that is used as a cooling lubricant.

18. A method for reducing growth of microbes which are harmful to the skin, comprising administering to the skin an effective amount of the composition of claim 1.

19. The method of claim 18, wherein the composition further contains at least one prebiotically active agent.

20. The method of claim 19, wherein the prebiotically active agent is a plant extract, glycerol monoalkyl ether, or ester of an organic acid.

21. The method of claim 19, wherein the microbes are coagulase-positive Staphylococcus spp., Propionibacterium acnes, Candida albicans, Malassezia furfur, Corynebacterium spp. or Peptostreptococcus spp.

22. The method of claim 21, wherein the Staphylococcus spp. is Staphylococcus aureus.

23. A method for reducing growth of microbes which are harmful to the skin, comprising administering to the skin an effective amount of the composition of claim 8.

24. The method of claim 23, wherein the composition further contains at least one prebiotically active agent.

25. The method of claim 24, wherein the prebiotically active agent is a plant extract, glycerol monoalkyl ether, or ester of an organic acid.

26. The method of claim 22, wherein the microbes are coagulase-positive Staphylococcus spp., Propionibacterium acnes, Candida albicans, Malassezia furfur, Corynebacterium spp. or Peptostreptococcus spp.

27. A method for reducing growth of microbes which are harmful to the mouth, comprising exposing the mouth or a dental prosthesis to an effective amount of the composition of claim 1.

28. The method of claim 27, wherein the composition further contains at least one prebiotically active agent.


SEQUENCE LISTING (13 sequences)

SEQ ID NO: 1 - protein, 40 aa, Artificial Sequence
  Synthetic construct
  MISC_FEATURE (8)..(8): Xaa is any essential or non-essential amino acid
  MISC_FEATURE (15)..(15): Xaa is any essential or non-essential amino acid
  MISC_FEATURE (20)..(20): Xaa is any essential or non-essential amino acid
  MISC_FEATURE (29)..(30): Xaa is any essential or non-essential amino acid
  MISC_FEATURE (34)..(34): Xaa is any essential or non-essential amino acid
  MISC_FEATURE (37)..(40): Xaa is any essential or non-essential amino acid
GIGDPVTXLKSGAIXHPVFXPRRYKQIGXXGLPXTKXXXX

SEQ ID NO: 2 - protein, 41 aa, Homo Sapiens
GIGDPVTCLKSGAICHPVFCPRRYKQIGTCGLPGTKCCKKP

SEQ ID NO: 3 - protein, 41 aa, Artificial Sequence
  Synthetic construct
GIGDPVTCLKSGAICHPVFCPRRYKQIGGCGLPGTKCCKKP

SEQ ID NO: 4 - protein, 65 aa, Homo Sapiens
MRIHYLLFALLFLFLVPVPGHGGIINTLQKYYCRVRGGRCAVLSCLPKEEQIGKCSTRGRKCCRR

SEQ ID NO: 5 - protein, 67 aa, Homo Sapiens
MRIHYLLFALLFLFLVPVPGHGGIINTLQKYYCRVRGGRCAVLSCLPKEEQIGKCSTRGRKCCRRKK

SEQ ID NO: 6 - protein, 4 aa, Artificial Sequence
  Synthetic construct
GQPR

SEQ ID NO: 7 - protein, 4 aa, Artificial Sequence
  Synthetic construct
GQRP

SEQ ID NO: 8 - protein, 4 aa, Artificial Sequence
  Synthetic construct
  MISC_FEATURE (1)..(1): N-palmitoyl
GQPR

SEQ ID NO: 9 - protein, 5 aa, Artificial Sequence
  Synthetic construct
KTTKS

SEQ ID NO: 10 - protein, 5 aa, Artificial Sequence
  Synthetic construct
  MISC_FEATURE (1)..(1): N-palmitoyl
KTTKS

SEQ ID NO: 11 - protein, 5 aa, Artificial Sequence
  Synthetic construct
  MISC_FEATURE (1)..(1): N-palmitoyl
YGGFM

SEQ ID NO: 12 - protein, 5 aa, Artificial Sequence
  Synthetic construct
  MISC_FEATURE (1)..(1): N-palmitoyl
YGGFL

SEQ ID NO: 13 - protein, 6 aa, Artificial Sequence
  Synthetic construct
  MISC_FEATURE (1)..(1): Palmitoyl
VGVAPG